(19) United States
(12) Patent Application Publication
(10) Pub. No.: US 2026/0258110 A1
(43) Pub. Date: Sep. 3, 2026

(54) VOLTAGE GATED SODIUM CHANNEL-SPECIFIC PEPTIDE APTAMERS AND USES THEREOF

(71) Applicant: The Medical College of Wisconsin, Inc., Milwaukee, WI (US)

(72) Inventors: Hongwei Yu, Milwaukee, WI (US); Seung Min Shin, Milwaukee, WI (US)

(21) Appl. No.: 19/474,400

(22) PCT Filed: Apr. 15, 2024
(86) PCT No.: PCT/US2024/024676

§ 371 Date: Oct. 10, 2025

Related U.S. Application Data
(60) Provisional application No. 63/495,946, filed on Apr. 13, 2023.

(51) Int. Cl. C07K 14/705; A61K 38/00; A61P 25/02; C12N 15/86

(52) U.S. Cl. CPC C07K 14/705; A61P 25/02; C12N 15/86; A61K 38/00; C12N 2750/14143

(57) ABSTRACT

Disclosed are polypeptide aptamers comprising peptide aptamers which functionally interact with Nay 1.7 voltage gated sodium channels, polynucleotides encoding the polypeptide aptamers, and infectious particles as well as methods of using the same in the treatment of diseases and disorders, for example, neuropathic pain.

### CROSS-REFERENCE TO RELATED APPLICATIONS

The present application claims priority to U.S. Provisional Patent Application No. 63/495,946 that was filed Apr. 13, 2023, the entire contents of which are hereby incorporated by reference.

### STATEMENT REGARDING FEDERALLY SPONSORED RESEARCH OR DEVELOPMENT

Not applicable.

### SEQUENCE LISTING

A Sequence Listing accompanies this application and is submitted as an xml file of the sequence listing named “650053_01054.xml” which is 93,688 bytes in size and was created on Apr. 15, 2024. The sequence listing is electronically submitted via Patent Center and is incorporated herein by reference in its entirety.

### BACKGROUND

Voltage-gated sodium channels (Navs) are key regulators of neuronal excitability and pain sensations (1). Mammals possess nine isoforms of Navs, of which Nav1.7, Nav1.8, and Nav1.9 are principally expressed in the primary sensory neurons (PSNs) of dorsal root ganglia (DRG) (2). The prominent roles of these Nav isoforms to human pain have been validated, e.g., a loss-of-function mutation in Nav1.7 (SCN9A) leads to congenital insensitivity to pain (CIP); conversely, gain-of-function mutations yields inherited erythromelalgia (IEM) and paroxysmal extreme pain disorder (PEPD), the conditions with symptoms at the opposite spectrum from those of CIP (2). Nav1.6, Nav1.1, and Nav1.3 are also expressed in PSNs and have been reported as possible targets for analgesics (3, 4). Currently, Nav1.7 is the leading target among Navs for developing analgesic therapies (5).

Numerous efforts have been made over the last decades to develop selective and efficacious Nav1.7 blockers to treat pain (6), but the success is limited. Most of the available small-molecule Nav1.7 blockers tested to treat pain are insufficient in target engagement, lack of targeting specificity or selective bioavailability in pain axis, and their global distribution contributes to cardiotoxicity, motor impairments, and CNS side-effects (6. 7). Development of biologics targeting Nav1.7 is an alternative growing trend (8-10) for analgesia. Nav1.7 neutralizing monoclonal antibodies have analgesic efficacy, but the results are not consistently replicated (11). Tarantula peptide Nav1.7 blockers (10, 12) are analgesic effectiveness but have poor membrane permeability, inadequate Nav1.7 selectivity, and short half-lives (6). Nav1.7-RNAi (6) and CRISPR-dCAS9 or ZEN epigenetic Nav1.7 suppression for analgesic gene therapy has been proposed (13), but these interventions at the mRNA and epigenetic levels lack the specificity of direct channel intervention, reducing safety and permitting off-target effects (6, 14, 15), and CAS9 immunity creates additional challenge for CRISPR gene therapies (16). Accordingly, there is a need in the art for improved analgesics and improved strategies to treat pain in affected subjects.

### SUMMARY

In an aspect of the current disclosure, polypeptide aptamers are provided. In some embodiments, the polypeptide aptamers comprise a sequence selected from the group consisting of SEQ ID NOs: 1-9, 50, and 52, or a sequence with at least 85% identity to one of SEQ ID NOs: 1-9, 50, and 52. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Nav1.7 channel protein.

In another aspect of the current disclosure, pharmaceutical compositions are provided. In some embodiments, the pharmaceutical compositions comprise a polypeptide aptamer comprising a sequence selected from SEQ ID NOs: 1-9, or a sequence with at least 85% identity to one of SEQ ID NOs: 1-9 and a pharmaceutically acceptable carrier or excipient. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Nav1.7 channel protein.

In another aspect of the current disclosure, polynucleotides are provided. In some embodiments, the polynucleotides comprise a nucleotide sequence encoding a polypeptide aptamer comprising a sequence selected from SEQ ID NOs: 1-9, or a sequence with at least 85% identity to one of SEQ ID NOs: 1-9. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Nav1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence.

In another aspect of the current disclosure, infectious particles are provided. In some embodiments, the infectious particles comprise a polynucleotide comprising a sequence encoding a polypeptide aptamer comprising a sequence selected from SEQ ID NOs: 1-9, or a sequence with at least 85% identity to one of SEQ ID NOs: 1-9. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Naᵥ1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence. In some embodiments, the infectious particle is a virus. In some embodiments, the virus is an adeno-associated virus (AAV). In some embodiments, the adeno-associated virus (AAV) is an AAV type 6 (AAV6).

In another aspect of the current disclosure, further pharmaceutical compositions are provided. In some embodiments, the pharmaceutical compositions comprise an infectious particle comprising a polynucleotide comprising a sequence encoding a polypeptide aptamer comprising a sequence selected from SEQ ID NOs: 1-9, or a sequence with at least 85% identity to one of SEQ ID NOs: 1-9. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Naᵥ1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence. In some embodiments, the infectious particle is a virus. In some embodiments, the virus is an adeno-associated virus (AAV). In some embodiments, the adeno-associated virus (AAV) is an AAV type 6 (AAV6).

In another aspect of the current disclosure, methods are provided. In some embodiments, the methods comprise contacting a cell with a polypeptide aptamer comprising a sequence selected from SEQ ID NOs: 1-9, or a sequence with at least 85% identity to one of SEQ ID NOs: 1-9 or an infectious particle comprising a polynucleotide comprising a sequence encoding a polypeptide aptamer comprising a sequence selected from SEQ ID NOs: 1-9, or a sequence with at least 85% identity to one of SEQ ID NOs: 1-9. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Naᵥ1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence. In some embodiments, the infectious particle is a virus. In some embodiments, the virus is an adeno-associated virus (AAV). In some embodiments, the adeno-associated virus (AAV) is an AAV type 6 (AAV6). In some embodiments, the method reduces peak Naᵥ1.7 current density in the neuron by at least 50% as measured by patch clamp electrophysiology. In some embodiments, the method does not change channel steady-state inactivation properties of Naᵥ1.7 channels in the neuron.

In some embodiments, methods of reducing or inhibiting stimulation of a neuron are provided. In some embodiments, the methods comprise contacting a neuron with a polynucleotide comprising a sequence encoding a polypeptide aptamer comprising a sequence selected from SEQ ID NOs: 1-9, or a sequence with at least 85% identity to one of SEQ ID NOs: 1-9 or an infectious particle comprising a polynucleotide comprising a sequence encoding a polypeptide aptamer comprising a sequence selected from SEQ ID NOs: 1-9, or a sequence with at least 85% identity to one of SEQ ID NOs: 1-9. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Naᵥ1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence. In some embodiments, the infectious particle is a virus. In some embodiments, the virus is an adeno-associated virus (AAV). In some embodiments, the adeno-associated virus (AAV) is an AAV type 6 (AAV6). In some embodiments, the method reduces peak Naᵥ1.7 current density in the neuron by at least 50% as measured by patch clamp electrophysiology. In some embodiments, the method does not change channel steady-state inactivation properties of Naᵥ1.7 channels in the neuron.

In some embodiments, further methods are provided. In some embodiments, the methods comprise administering a pharmaceutical composition an infectious particle comprising a polynucleotide comprising a sequence encoding a polypeptide aptamer comprising a sequence selected from SEQ ID NOs: 1-9, or a sequence with at least 85% identity to one of SEQ ID NOs: 1-9 to a subject.

In some embodiments, methods of treating neuropathic pain in a subject in need thereof are provided. In some embodiments, the methods comprise administering a therapeutically effective amount of a pharmaceutical composition comprising an infectious particle comprising a polynucleotide comprising a sequence encoding a polypeptide aptamer comprising a sequence selected from SEQ ID NOs: 1-9, or a sequence with at least 85% identity to one of SEQ ID NOs: 1-9 to the subject to treat neuropathic pain in the subject. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Naᵥ1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence. In some embodiments, the infectious particle is a virus. In some embodiments, the virus is an adeno-associated virus (AAV). In some embodiments, the adeno-associated virus (AAV) is an AAV type 6 (AAV6). In some embodiments, administration comprises local delivery of the pharmaceutical composition. In some embodiments, local delivery of the pharmaceutical composition comprises delivery to a dorsal root ganglion in the subject. In some embodiments, the method reduces mechanical or cold sensitization in the subject. In some embodiments, the subject is a human subject. In some embodiments, the subject is suffering from chronic pain. In some embodiments, the subject is suffering from neuropathic pain or neurogenic pain. In some embodiments, the subject has been diagnosed with osteoarthritis.

In some embodiments, methods of treating pain due to a traumatic nerve injury in a subject in need thereof are provided. In some embodiments, the methods comprise administering a therapeutically effective amount of a pharmaceutical composition comprising an infectious particle comprising a polynucleotide comprising a sequence encoding a polypeptide aptamer comprising a sequence selected from SEQ ID NOs: 1-9, or a sequence with at least 85% identity to one of SEQ ID NOs: 1-9 to the subject to treat the pain due to the traumatic nerve injury in the subject. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Naᵥ1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence. In some embodiments, the infectious particle is a virus. In some embodiments, the virus is an adeno-associated virus (AAV). In some embodiments, the adeno-associated virus (AAV) is an AAV type 6 (AAV6). In some embodiments, administration comprises local delivery of the pharmaceutical composition. In some embodiments, local delivery of the pharmaceutical composition comprises delivery to a dorsal root ganglion in the subject. In some embodiments, the method reduces mechanical or cold sensitization in the subject. In some embodiments, the subject is a human subject. In some embodiments, the subject is suffering from chronic pain. In some embodiments, the subject has been diagnosed with osteoarthritis.

In another aspect of the current disclosure, cells are provided. In some embodiments, the cells comprise a heterologous polynucleotide encoding a human Naᵥ1.8 protein. In some embodiments, the human Naᵥ1.8 protein has the amino acid sequence SEQ ID NO: 20. In some embodiments, the heterologous polynucleotide encoding a human Naᵥ1.8 protein comprises SEQ ID NO: 21. In some embodiments, the heterologous polynucleotide further encodes a human Naᵥb2 protein. In some embodiments, the Naᵥb2 protein has the sequence SEQ ID NO: 24. In some embodiments, the heterologous polynucleotide further comprises a self-cleaving peptide sequence, a furin cleavage site, or both a self-cleaving peptide sequence and a furin cleavage site. In some embodiments, the polynucleotide encoding a human Naᵥ 1.8 protein comprises SEQ ID NO: 22. In some embodiments, the cell is a human cell. In some embodiments, the cell is human embryonic kidney cell.

In another aspect of the current disclosure, kits, systems, or platforms are provided. In some embodiments, the kits, systems, or platforms comprise a polypeptide aptamer comprising a sequence selected from SEQ ID NOs: 1-9, or a sequence with at least 85% identity to one of SEQ ID NOs: 1-9 or a polynucleotide comprising a sequence encoding a polypeptide aptamer comprising a sequence selected from SEQ ID NOs: 1-9, or a sequence with at least 85% identity to one of SEQ ID NOs: 1-9; and reagents for performing electrophysiology experiments. In some embodiments, the sequence is SEQ ID NO: 1, 4, or 6, or a sequence with at least 85% identity to one of SEQ ID NO: 1, 4, or 6. In some embodiments, the sequence is SEQ ID NO: 1, or a sequence with at least 85% identity to SEQ ID NO: 1. In some embodiments, the sequence is SEQ ID NO: 1. In some embodiments, the polypeptide aptamer further comprises a detectable marker. In some embodiments, the detectable marker comprises a fluorescent protein. In some embodiments, the fluorescent protein comprises green fluorescent protein. In some embodiments, the polypeptide aptamer further comprises a linker. In some embodiments, the linker comprises SEQ ID NO: 10. In some embodiments, the polypeptide aptamer further comprises a detectable marker and a linker, wherein the detectable marker, linker, and sequence are a single continuous peptide sequence and are configured, from N-terminus to C-terminus: detectable marker, linker, sequence. In some embodiments, the polypeptide aptamer binds to human Naᵥ1.7 channel protein. In some embodiments, the sequence encoding the polypeptide aptamer comprises one of SEQ ID NOs: 11-19. In some embodiments, the polynucleotides further comprise a promoter or enhancer, wherein the nucleotide sequence is operably linked to the promoter or enhancer. In some embodiments, the promoter is a hybrid human cytomegalovirus (CMV) enhancer/chicken β-actin (CBA) promoter. In some embodiments, the polynucleotides further comprise at least one adeno-associated virus (AAV) inverted terminal repeat (ITR) sequence. In some embodiments, the electrophysiology experiments comprise patch clamp electrophysiology.

In some embodiments, the kits, systems, or platforms comprise a polypeptide aptamer comprising a sequence selected from SEQ ID NOs: 1-9, or a sequence with at least 85% identity to one of SEQ ID NOs: 1-9 or a polynucleotide comprising a sequence encoding an sequence selected from SEQ ID NOs: 1-9, or a sequence with at least 85% identity to one of SEQ ID NOs: 1-9, and instructions for use in treating a subject suffering from neuropathic pain.

### BRIEF DESCRIPTION OF THE FIGURES

FIGS. 1A, 1B, 1C, 1D, 1E, 1F, 1G, 1H, 1I, and 1J. In silico prediction of Naᵥ1.7-IDRs and design of candidate Naᵥ1.7iPAs (1.7iPAs). Diagram of rat Naᵥ1.7 protein, with white boxes labeling DI-DIV of Naᵥ1.7 (A) and the red bars below showing position of the predicted iPAs (B). Consensus prediction of IDRs by DEPICTER (C). The phosphorylation sites were predicted by DEPP (D). Nine candidate iPAs with their aa sequences, position in Naᵥ1.7, and IDR scores, corresponding to SEQ ID NOs: 1-9, from top to bottom. (E). A map showing each component of an AAV plasmid coding GFP-iPA, a black line pointing to iPAs (F). The structure analysis of GFP-fused 1.7iPA1 by I-TASSER, top panel: structure of free 1.7iPA1 (G). Images (GFP, left; phase, middle; and merged pictures, right) show expression of constructs carrying 1.7iPA1-4 and 6 after transfection to HEK cells (H). Scale bar: 25 μm for all. GFP and Gapdh western blots of the cell lysates after transfection with 1.7iPA1-4 and 6 to HEK cells (I). Initial screening of nine iPA on INa by whole-cell patch-clamp recording as described in Methods after transfection into HEK1.7 cells. * and *** denote p<0.05 and 0.001, respectively; one-way ANOVA and Tucky post hoc (J).

FIGS. 2A, 2B, 2C, 2D, 2E, and 2F. Confirmation of INa1.7 inhibition by 1.7iPA1, 4, and 6 and gating kinetics. (A) Representative traces of INa1.7 by whole-cell patch-clamp recording from sham (transfection without plasmid), GFP, 1.7iPA3 (NP), 1.7iPA1, 1.7iPA4, and 1.7iPA6 transfected HEK1.7 cells. Inserts: recording protocol and current/time scales. Summary of the confirmation tests of candidate iPAs expression in HEK1.7 cells in (B) comparison of corresponding mean peak current density-voltage (I/V) relationship from different constructs as indicated and (C) quantitative analysis of averaged peak INa1.7 density; ***p<0.001, one-way ANOVA followed by Tukey post hoc. No effects of expression of GFPiPA1, GFPiPA4, and GFPiPA6 were observed on steady-state activation (D, inset: V1/2 activation) and fast inactivation (E, inset: V1/2 inactivation), compared to naive and GFP or NP-transfected HEK1.7 cells. NaviPA1 is highly conserved in rat, mouse, and human, SEQ ID NOs: 30-32, from top to bottom (F). Black and yellow asterisks at the bottom denote positively and negatively charged aa; the red and blue asterisks on the top denote known lysine acetylation and serine phosphorylation sites, and IDR scores and % of positively (+) and negatively (−) charged aa were shown at the right sides of the alignment.

FIGS. 3A, 3B, 3C, 3D, 3E, 3F, and 3G. Sodium channel specificity of NaviPA1 (1.7iPA1) inhibition. The aa sequence alignment of 1.7iPA1 with the corresponding sequences of TTXs Nav1.6, Nav1.3, Nav1.1 SEQ ID NOs: 1 and 33-35. (A), as well as TTXr Nav1.5, Nav1.8, and Nav1.9 SEQ ID NOs: 1 and 36-38. (B) of rat specie. The homologous aa (identity and similarity) was highlighted in heavy or light black shadows and % of identical or similar aa shown at the right sides of the alignments. (C-G) Panels from left to right show the comparisons of INa traces in presence of 1.7iPA1 in HEK1.1, 1.3, 1.6, 1.5, and 1.8 cells (insert: pulse protocol and scale); peak INa density (p<0.001, one-way ANOVA and Turkey post hoc), I/V curves, steady-state activation (insert: V1/2 activation) and fast inactivation kinetics (insert: V1/2 inactivation).

FIGS. 4A, 4B, 4C, 4D, 4E, 4F, 4G, and 4H. NaviPA1 on I_{Na} of rat DRG neurons (male) and hiPSC-SNs (female). (A-C) Panels from top to bottom illustrate representative traces and averaged peak I_{Na} densities of total I_{Na} (A), TTXs I_{Na} (B), and TTXr I_{Na} (C) recorded from sensory neurons (diameter≤35 μm) dissociated from naïve male rats subjected with (panels from left to right) sham (surgical exposure without injection), and 4 wk after L4/L5 DRG injected with AAV6-encoded GFP, GFPNP, and GFPNaviPAI. Inserts: representative PSN images (scale bars 25 μm for all) of each group, current/time scales, and recording pulse protocol. (D, E) Representative montage ICC images illustrate hiPSC-SNs at DIV25 after transduction with LV-GFPNP (D) and LV-GFPNaviPA1 (E) at equal MOI=5. (F-H) illustrate representative traces and averaged peak I_{Na} densities of Total I_{Na} (F), TTXs I_{Na} (G), and TTXr I_{Na} (H) recorded from hiPSC-SNs (DIV25) expressing NP and NaviPA1. Inserts: current/time scales and recording pulse protocol. *, **, and *** denote p<0.05, 0.01, and p<0.001, one-way ANOVA and Turkey post hoc.

FIGS. 5A, 5B, 5C, 5D, 5E, 5F, 5G, 5H, 5I, and 5J. NaviPA1 binds to full-length Nav1.7 protein and phosphoinositides. (A) Immunoblots (IB) show selectivity of Nav1.7 antibody using cell lysates from naïve HEK cells, and HEK1.5, HEK1.7, HEK1.6, HEK1.1, HEK1.3, CHO1.8 cells, and 50B11 cells. (B-E) Representative IHC images show Nav1.7 detection (red) in SDH (red), sciatic nerve (green), DRG neurons (red), and cutaneous nerve fibers (red). Scale bars: 100 μm. (F) IBs of Nav1.7, GFP, NKA, NKAla, Gapdh in the cytosol and membrane samples extracted from HEK1.7 cells transfected with sham (transfection without plasmid), GFP, GFPNaviPA1, and GFP1.7iPA2. (G) Nav1.7 IB (left) and silver stain (right) of inputs (cell lysates, 20 μg for each lane) and pulldown beads (10 μL for each lane) prepared by a ‘nondenaturing’ lysis buffer from HEK1.7 cells transfected with GFP, GFPNaviPA1, and GFP1.7iPA2. SEQ ID NOs: 39-46, from top to bottom. (H) Stained gel pieces ranging 100-300 kDa (G, red asterisk denotes Nav1.7 site) from GFPNP and GFPNaviPA1 excised for mass spectrometry showing detection of unique human Nav1.7 peptides (red) in GFPNaviPA1 pull-down sample. Silver stain on ID SDS-PAGE gel of GFP-affinity pulldown beads in the NG108-15 cells transfected with GFPNaviPA1 and GFP and cell lysates prepared using denaturing RIPA buffer (I) and the results of PIP strip analysis (J). LPA, Lysophosphatidic acid; LPC, Lysophosphocholine; PtdIns, Phosphatidylinositol; PtdIns (3) P, Phosphatidylinositol (3) phosphate; PtdIns (4) P, Phosphatidylinositol (4) phosphate; PtdIns (5) P, Phosphatidylinositol (5) phosphate; PE Phosphatidylethanolamine; PC, Phosphatidylcholine; SIP, Sphingosine 1-Phosphate; PtdIns (3,4)P₂, Phosphatidylinositol (3,4) bisphosphate; PtdIns (3,5)P₂, Phosphatidylinositol (3,5) bisphosphate; PtdIns (4,5)P₂, Phosphatidylinositol (4,5) bisphosphate; PtdIns (3,4,5) P3, Phosphatidylinositol (3,4,5) trisphosphate; PA, Phosphatidic acid; PS, Phosphatidylserine.

FIGS. 6A, 6B, 6D, 6E, 6F, 6G, and 6H. Define polybasic NLS and adjacent serine in NaviPA1. (A) Sequence alignments of NaviPA1, mutant 1 (mt) with alanine substitution of ten serine residues, mt2 by alanine substitution of arginine/lysine (R/K) (mt2) within predicted NLS domain, and mt3-6 with alanine substitution of bi- or tri-serine residues at different serine sites, as indicated. SEQ ID NOs: 1 and 47-52, from top to bottom. (B-E) ICC comparison of GFP signals 48 hours after plasmids coding NaviPA1, GFPNP, mt1, and mt2 transfected into HEK1.7 cells. (F) Representative immunoblots of endogenous Nav1.7, as well as GFPNP, NaviPA1, mt1, and mt2, in extracted cytosol, membrane, and nuclear samples after transfection into HEK1.7 cells. Cytosol, membrane, and nuclear loading were indicated by GAPDH, NKAla, and LamBI, respectively. (F1) Quantitative comparison of membrane binding and nuclear entry of NaviPA1, mt1, and mt2 after transfection, * and ** denote p<0.05 and 0.01, one-way ANOVA and Tucky post hoc. (G) Representative I_{Na1.7} traces of HEK1.7 cells recorded from sham, GFPNP, NaviPA1, mil-mt6 (3-4 days after transfection), as indicated. (H) Quantification summary of peak I_{Na} densities; * **, and *** denote p<0.05, <0.01, and <0.001; one-way ANOVA and Tukey post hoc.

FIGS. 7A, 7B, 7C, 7D, 7E, 7F, 7G, 7H, 7I, and 7J. Treatment of established neuropathic pain by DRG AAV6-NaviPA1 (male rats). Purified AAVs (A, silver stain) were prepared for the experiment in an animal protocol schematically outlined (B). The time courses (C-F) of vF, Pin, Heat, and Cold before and after DRG injection of either AAV6-NaviPA1 (n=7) or AAV6-NP (control, n=8). For the treatment data, we used the measures on the 14^{th} day after TNI and before AAV treatment (tBL) as the peak pain intensity (100%), and the measures of each sensory modality after treatment were normalized to the measures at the tBL and the percentage of pain relief for each modality at multiple time points was calculated (FIG. 1C1-F1). *p<0.05, **p<0.0,1 and ***p<0.001 for comparisons to the tBL within group and #p<0.05, **p<0.01, and ***p<0.001 between groups. Repeated measures two-way ANOVA for vF and Heat, and Tukey (within group) and Bonferroni (between group) post hoc; and non-parametric Friedman ANOVA for Pin and Cold tests and Dunn's post hoc. Summed average pain relief in the 6-week treatment course showed 52%, 49%, 69%, and 67% reduction of vF-, Pin-, Cold-, and Heat-stimulated mechanical and thermal pain behaviors, respectively (FIG. G). **p<0.01 and p<0.001, unpaired, two-tailed student's t-test. (H) Results of CPP scores (seconds, s) of pre-conditioning chamber and of the GBP-paired chamber between AAV-NaviPA1 (n=7) and AAV-NP (control, n=8), ***p<0.001 (unpaired, two-tailed Student's t-test)

FIGS. 8A, 8B, 8C, 8D, 8E, 8F, and 8G. IHC of GFP-NaviPA1 and target gene expression. (A-D) Representative IHC montage images (GFPNaviPA1 with Tubb3) show neuronal expression profile 6 weeks after AAV-NaviPA1 injection in TNI rats (A), colocalization of GFP-NaviPA1 with Nav1.7 and Nav1.6 positive neurons (B, C), but not with GFAP positive perineuronal glia (D, the square region was enlarged and montage images shown as D1). (E-G) Representative IHC montage images illustrate GFPNaviPA1 (green) and Nav1.7 (red) in PSN central terminals of ipsilateral spinal dorsal horn (E), GFPNaviPA1 (green) and Tubb3 (red) in sciatic nerve (F), and GFPNaviPA1 (green) and NF200 (red) in PSN peripheral terminals of skin section (G). Scale bar (μm): A, 200; B, C, D and D1, 100; E, 200; G and G, 50 μm.

FIGS. 9A, 9B, 9C, 9D, 9E, and 9F. NaviPA1 expression on neuronal excitability of rat PSNs (male). (A, B) Representative AP traces elicited by 250 ms depolarizing current of 180 pA (A) and 280 pA (B) (same cells) from RMP were recorded from DRG neurons dissociated from the rats of sham, TNI only, and GFP-expressing neurons in TNI treated with AAV6-NP or AAV6-NaviPA1, as indicated. (C) Comparison of responses (number of APs evoked by a 250 ms stimulus) for the populations of DRG neurons in different groups across a range of step current injections from 100 to 280 pA; ***p<0.001, two-way ANOVA of main effects of groups with Bonferroni post-hoc. Scatter plots with bars show analysis of the rheobases (D) and AP numbers evoked by input current at 180 pA (E) and 280 pA (F) from RMP, respectively. The number in each group is the number of analyzed neurons per group. *, and *** denote p<0.05 and <0.001, respectively, One-way ANOVA and Turkey post-hoc.

FIGS. 10A, 10B, 10C, 10D, 10E, 10F, 10G, and 10H. Analgesia of DRG-AAV6-NaviPA1 treatment in female TNI rats. Analogous figures to FIG. 7 show significant analgesia (A-D) and % of pain reduction (A1-D1) after DRG delivery of AAV6-NaviPA1 in the established TNI pain behaviors of female rats. *, **, and *** denote p<0.05, 0.01, and 0.001 for comparisons to the treatment baseline (tBL) within group and #p<0.05, ##p<0.01, and ###p<0.001 for comparisons between groups. Repeated measures parametric two-way ANOVA for vF and Heat followed by Tukey (within group) and Bonferroni (between group) post hoc; and non-parametric Friedman ANOVA for Pin and Cold tests and Dunn's post hoc. Right panels of A2-D2 show average pain relief of each modality in 3.5-month treatment, ***p<0.001; comparisons between groups (unpaired, two-tailed Student's t tests). CPP difference scores(s) of pre-conditioning chamber and of the GBP-paired chamber between AAV-NaviPA1 (n=8) and AAV-NP (control, n=8), *p<0.01 (unpaired, two-tailed Student's t test) (E). Representative montage IHC images colocalization of GFP-NaviPA1 with Tubb3 (F), Nav1.7 (G), and Nav1.6 (H) show neuronal expression profile 6 weeks after AAV-NaviPA1 injection. Scale bar: 100 μm for F-H.

FIGS. 11A, 11B, 11C, 11D, and 11E. In silico DEPICTER and I-TASSER. (A) IDRs are predicted by DEPICTER (3), a prediction algorithm that aggregates results from the multiple servers (IUPred-L and S: Prediction of Intrinsically Unstructured Proteins. SPOT-Disorder-Single: Accurate Single-Sequence Prediction of Protein Intrinsic Disorder. Disco Protein Binding: Prediction of IDR Protein Binding. fMoRFpred: Fast Molecular Recognition Feature predictor. DisoRDPbind: Predictor of disorder-mediated RNA, DNA, and protein binding regions. Anchor 2: Potential binding sites in disordered regions. DiscoRNA (DNA) Bind: Prediction of Potential RNA (DNA) binding regions. Disordered: Flexible Linker predictor. DiscoMultipleFunction: Annotations of disordered multifunctional residues), with each algorithm indicated on the left side. (B-E) The crystal structure analysis of GFP1.7iPA2, 3, 4, and 6 by I-TASSER, as indicated.

FIGS. 12A, 12B, 12C, 12D, and 12E. Establishment of Nav1.8 stable expression system based on HEK cells. (A) Comparison of I/V curves between HEK1.7 and HEK1.8 cells. (B and C) Voltage-dependent activation and steady-state fast inactivation curves recorded from HEK1.8 cells, HEK1.7 cells, and HEK1.5 cells. Insert: recoding protocol. (D) Comparison of I_{Na1.8} inhibition by TTX (1 to 30 μM) of HEK1.8 cells and (E) I_{Na1.7} inhibition by TTX 0.1 in HEK1.7 cells. ** and *** denote p<0.01 and 0.001, student t test (F and L), and one-way ANOVA and Tucky post hoc (K) . . .

FIGS. 13A, 13B, 13C, 13D, and 13E. Lack of NaviPA1 effect on I_{Na1.8} transiently expressed in ND7/23 neuronal cells. Representative I_{Na1.8} currents recorded from Nav1.8+GFPNP and Nav1.8+NaviPA1 transfected cells (A). Scatter plot of peak current density of Nav1.8+GFPNP (n=21) and Nav1.8+NaViPA1 (n=22) transfected cells (B). I/V curves from Nav1.8+GFPNP and Nav1.8+NaViPA1 transfected cells (C). Comparison of the voltage-dependence of activation and inactivation for Nav1.8+GFPNP and Nav1.8+NaViPA1 transfected cells (D). Comparison of recovery from inactivation for Nav1.8+ GFPNP and Nav1.8+NaviPA1 transfected cells (E). For this set of experiments, the pipette solution contained (mM): CsCl 140, NaCl 10, EGTA 1.1, HEPES 10, pH7.30 and the bath solution contained (mM): NaCl 140, TEA-CI 20, KCl 3, CaCl2 1, MgCl2 1, HEPES 10, pH7.30, with 1 μM TTX.

FIGS. 14A, 14B, 14C, 14D, 14E, 14F, 14G, and 14H. NaviPA1 on I_{Na1.7} of NG108 and F11 cells, IKv of NG10⁸ cells, and HVA ICa of DRG neurons. Representative I_{Na1.7} single traces at 0 mV recorded from sham-, GFPNP-, NaviPA1-transfected cells, merged I_{Na1.7}, I/V curves with curve of TTX (0.1 μM), and peak I_{Na1.7} density from NG108-15 cell (A-C) and F11 cells (D-F). Insets: protocol and current/time scales. ***p<0.001, one-way ANOVA and turkey post hoc. (G) Representative I_{Kv} of sham-NG10⁸ cells showing I_{Kv} defined by outward currents blocked by Tetraethylammonium (TEA, 5 mM) or NG10⁸ cells transfected with GFP, 1.7NP, and NaviPA1 (G1-G5). insets: recording protocol and current/time scales. I_{Kv} density-voltage (I/V) curves (G6) and quantitative analysis of peak I_{Kv} density (G7), p>0.05, one-way ANOVA and Tukey post hoc. (H) DRG neuron HVA ICa recording. Typical HVA ICa trace in a small-sized neuron from a naïve rat shows a threshold for activation around −30 mV and a maximum current amplitude activation at −10 mV, displaying small inactivation (H). Typical traces of HVA I_{Ca} recorded at −10 mV of neurons from a sham-operated rat (H1) and naïve rats injected with AAV6-GFP (H2), −1.7NP (H3), and -NaviPA1 (H4). HVA I_{Ca} density-voltage (I/V) curves (H5) and averaged peak HVA I_{Ca} density (H6), p>0.05, one-way ANOVA and Tukey post hoc.

FIGS. 15A and 15B. TTXs and TTXr I_{Na} recording in DRG neurons from naïve rats. (A-A2) Representative traces of voltage-gated total, TTXr (bath TTX 1.0 μM in the same cells), and TTXs (total I_{Na} subtracts TTXr I_{Na}) I_{Na} recorded from small-sized DRG neurons. Inserts: scales, voltage protocol, and quantification of peak I_{Na} densities. (B-B2) Representative traces of voltage-gated total, TTXs, and TTXr I_{Na1.8}-like (manipulated by a voltage protocol) recorded from small-sized DRG neurons. Protocol for separation of TTXr and TTXs I_{Na}: A 500 ms prepulse to −120 or −50 mV was applied before a 50 ms test pulse from −100 to +40 mV with steps of 10 mV (inset). Both TTXs and TTXr I_{Na} were apparent after the −120 mV prepulse (top traces); only TTXr I_{Na} were obtained after the −50 mV prepulse (bottom traces), and the TTXs component was obtained (middle traces) by digitally subtracting the TTXr I_{Na} from the total I_{Na}. (B3) Average peak I_{Na} density-voltage relationships for total, TTXs, and TTXr I_{Na} of small DRG neurons. Smooth lines are I-V curves generated using the Boltzmann fit parameters of the respective activation curves. Averaged peak I_{Na} densities (B4) and normalized peak I_{Na} densities (B5) of total, TTXs, and TTXr I_{Na}.

FIG. 16. Lentivector construct to express NaViPA and NP.

FIGS. 17A, 17B and 17C. I_{Na}, BK I_{Kv}, and HVA I_{Ca} recording from hiPSC-SNs. (A) Representative traces of total I_{Na}, TTXr I_{Na} (1 μM TTX in bath solution), TTXs I_{Na}, recorded from naïve hiPSC-SNs (DIV25, Inserts: protocol and scales), and quantitative analysis of peak currents. A subtraction protocol is used to separate TTXr and TTXs I_{Na}. (B) Representative traces of BK I_{Kv} recorded from hiPSC-SNs (DIV21) in naïve, TEA in bath solution, NaviPA1- and NP-expressing hiPSC-SNs, and quantitative analysis of peak BK I_{Kv} density (panels from left to right). No difference, one-way ANOVA and post hoc. (C) Representative traces of HVA I_{Ca} recorded from hiPSC-SNs (DIV21) in naïve, NaviPA1- and NP-expressing hiPSC-SNs, and quantitative analysis of peak HVA I_{Ca} density (panels from left to right). No difference, one-way ANOVA and post hoc.

FIG. 18. No effects of NaviPA1mt1 and 2 on I_{Na} in HEK1.8 cells. Representative I_{Na} traces recorded from naïve and transfected HEK1.8 cells and peak current densities, as indicated.

FIGS. 19A, 19B, 19C, 19D, 19E, and 19F. In vivo pilot analgesic testing of NaviPA1. (A-C) Time courses for the group averages of sensitivity to vF, Pin, and Cold after DRG injection of either AAV6-NaviPA1 (n=5) or AAV6-NP (control, n=5) and subsequent TNI induction 3-wk after AAV injection. $ denotes p<0.001, compared between tBL and 1 week after injection. **p<0.05 and ***p<0.001 (A) indicate comparisons to 1-week after AAV injection within groups, and ##p<0.01 and ###p<0.001 for comparisons between groups. Repeated measures parametric two-way ANOVA for vF and Heat followed by Tukey (within group) and Bonferroni (between group) post hoc; and non-parametric Friedman ANOVA for Pin and Cold tests and Dunn's post hoc. Right panels of A-C show TNI tAUC calculated using measures 35-day post AAV; *p<0.05 and **p<0.01, unpaired, two-tailed Student's t-test for vF, and Mann-Whitney U tests for Pin and cold). Representative montage IHC images of DRG section (D, co-labeled GFP with Tubb3, showing colocalization in merged image), ipsilateral (ipsi.) hindpaw skin section (E) with dashed lines demarcating the dermis (De) and epidermis (Epi) boundaries, and spinal cord section (F) co-labeled GFP with CGRP (red), showing colocalization in merged image (arrowheads point to reduced CGRP innervation in ipsi. DH).

FIGS. 20A, 20B, 20C, 20D, 20E, 20F, 20G, and 20H. Establishment of HEK-Nav1.8 stable expression system. (A) An expression plasmid of pcDNA3.1 (+)-SCN10A-Furin-P2A-SCN2B. (B-D) Immunoblots of cell lysates verify stable expression of Nav1.8a (B) and Na2b (C), both are highly enriched in plasm membrane (D). Traces of inward I_{Na1.8} by whole-cell voltage-clamp recording (E), A803467 responses (F), and comparison of single traces (+10 mV) recorded from HEK1.8 cells and Cho1.8 cells (G). (H) Comparison of I/V curves between HEK1.7 and HEK1.8 cells.

FIGS. 21A, 21B, and 21C. 1.7/1.8iPA inhibits both I_{Na1.7} and I_{Na1.8}. (A-C) Transfection of 1.7/1.8iPA into NG10⁸ cells has no effects on BK channels (bath containing 2 mM CaCl2). *, **, and *** denote p<0.05, 0.01, and 0.001, one-way ANOVA and Tukey post hoc.

FIGS. 22A, 22B, 22C, 22D, and 22E. 1.7/1.8iPA and sequence alignments. (A) Nav1.7 (top) intracellular segment IDRs (bottom, grey areas). Location of 1.7/1.8iPA (red). (B-D) Conservation of AnkG and Pdzd2-I domains (red squares) within 1.7/1.8iPA sequence in rodent and human (B) SEQ ID NOs: 54-56, among TTXs Navs (C) SEQ ID NOs: 58-61, and between Nav1.7 and TTXr Navs (D) SEQ ID NOs: 62-67; * on top of aa sequence denote CK2 phosphorylation sites that enhance the binding to AnkG; and IDR scores, % of identity or similarity and negative (−) aa are shown on the right sides of alignments. (E) Analogous AnkG binding aa sequence in 1.7/1.8iPA and Kv7.2/7.3, SEQ ID NOs: 68-71.

FIGS. 23A, 23B, and 23C. Kv7.2/7.3 M current recording (NG10⁸ cells). M currents are elicited by 300 ms depolarizing steps ranging −80 mV to +120 mV in 10 mV increments with 5s intervals between steps from a holding potential of −80 mV.^{37, 43} (A-C) M current traces (A, XE991, a selective I_{Kv}7.2/7.3 inhibitor), I/V curves (B), and peak I_{Kv}7.2/7.3 density (C) with and without XE991. *p<0.05 and ***p<0.001, One-way ANOVA and Tukey post hoc.

FIG. 24. Design of site-directed mutagenesis. 1.7/1.8iPA and mutant 1.7/1.8iPA sequences with boxes indicating AnkG and Pdzd2-I domains; E, key aa for AnkG binding; and *pointing CK2 phosphorylation sites, SEQ ID NOs: 57 and 72-75.

FIG. 25. AAV plasmid expressing 1.7//1.8iPA. DNA sequence of 1.7/1.8iPA is cloned with a linker (GLRSRAQASNSAVDGTAGPGS) to form a chimeric monoGFP-linker-1.7/1.8iPA orientation transcribed by CBA promoter.

FIGS. 26A, 26B, 26C, and 26D. (A) Nav1.7 (top) intracellular segment IDRs (bottom, grey areas). Location of 1.7/1.8iPA (red). Expression of 1.7/1.8iPA (GFP-fusion) in HEK1.7 (B) and HEK1.8 (C) cells inhibit I_{Na1.7} and I_{Na1.8}. (D) AnkG and Pdzd2-I domains within 1.7/1.8iPA sequence (rectangles), SEQ ID NO: 53.

FIG. 27. Sequence annotation of pCMV-cDNA3.1 (+)-hSCN10A-FurinP2A-hSCN2B.

### DETAILED DESCRIPTION

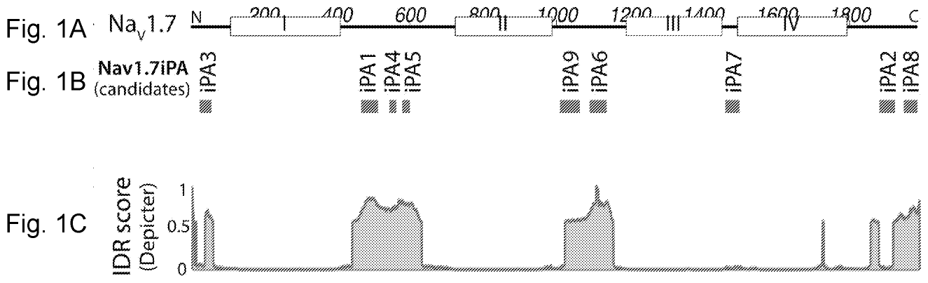

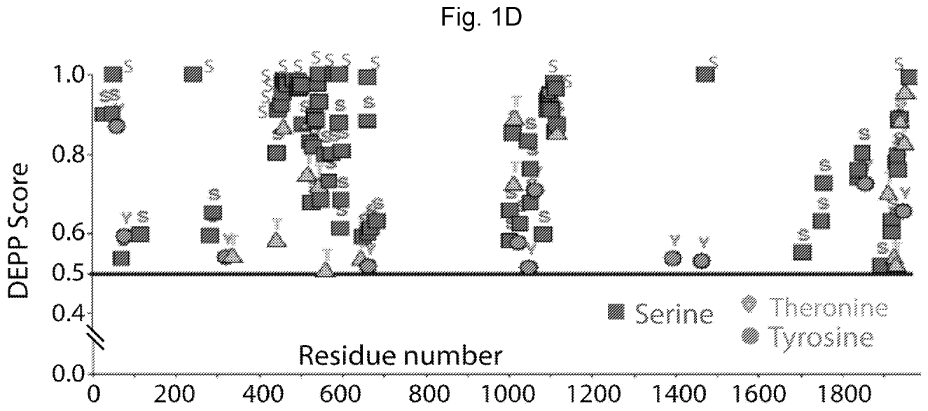

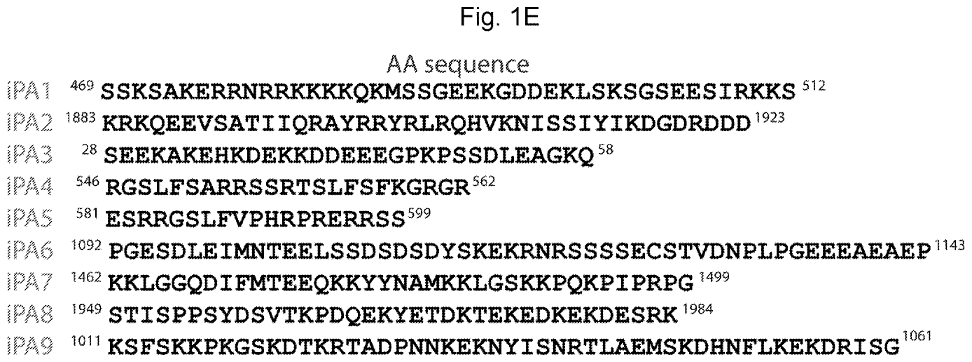

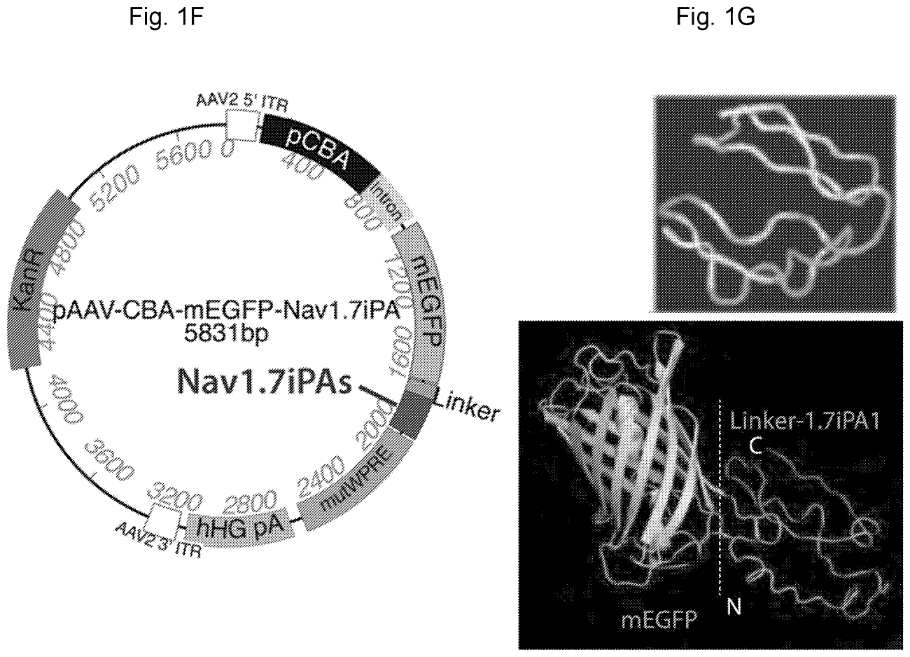

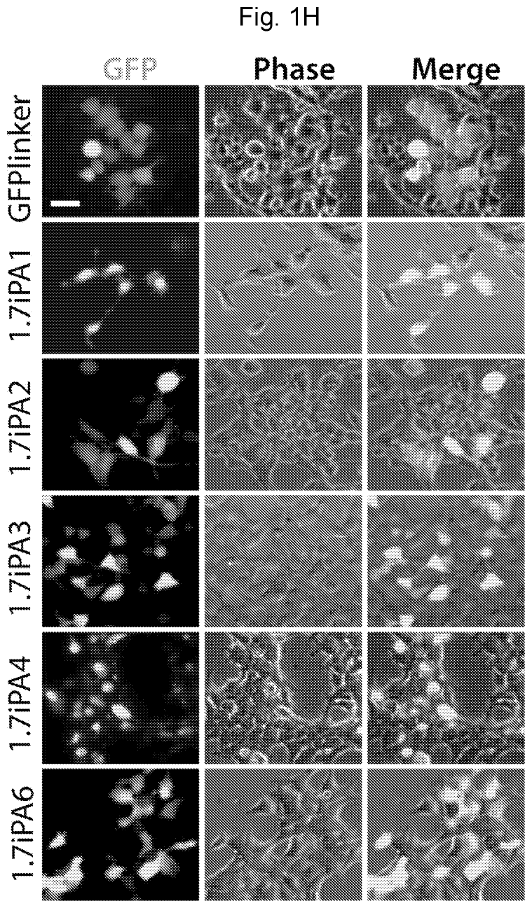

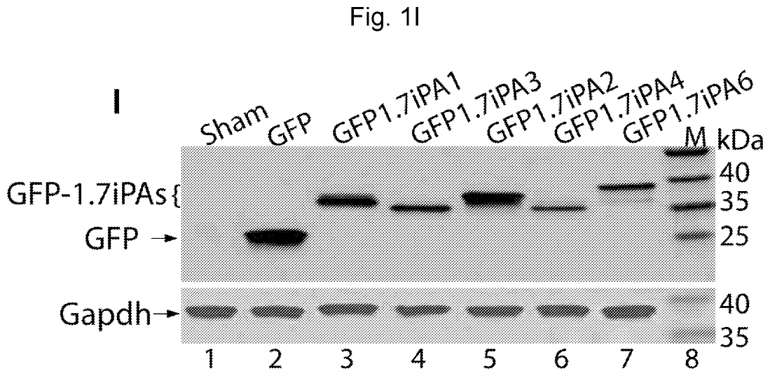

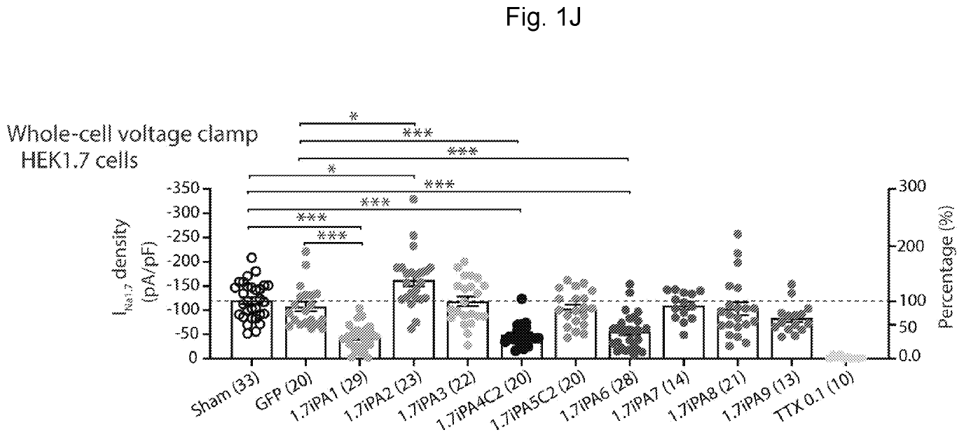

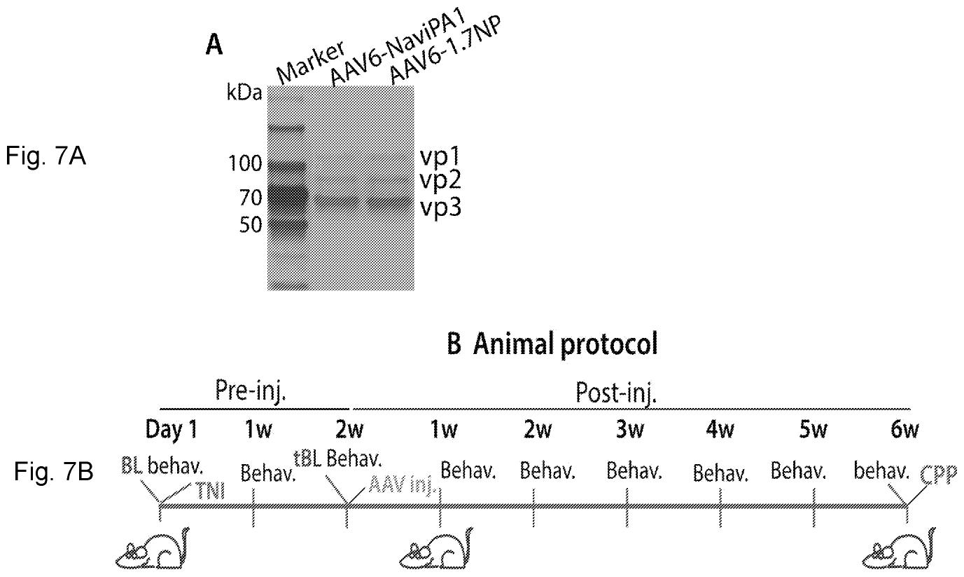

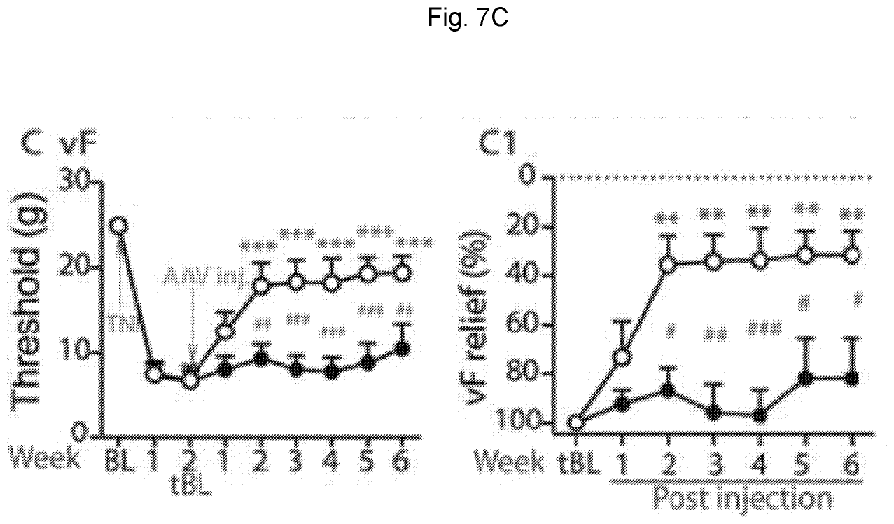

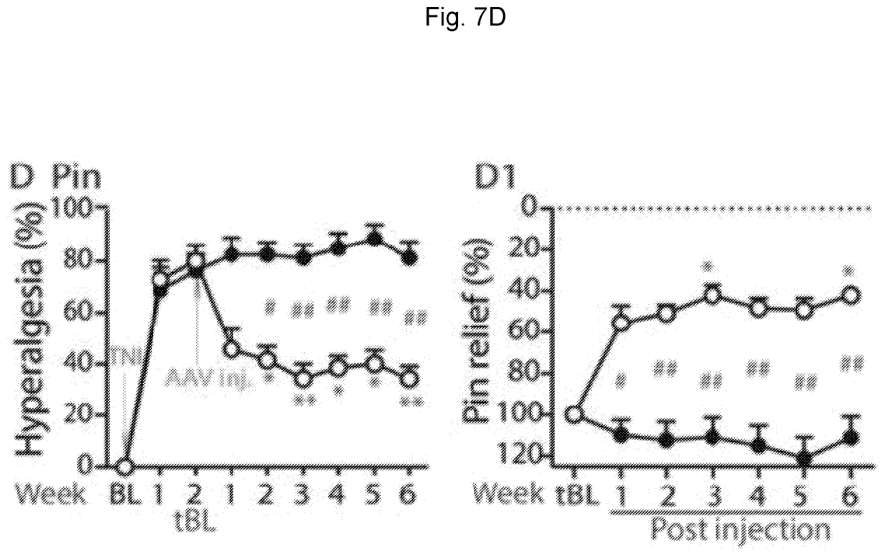

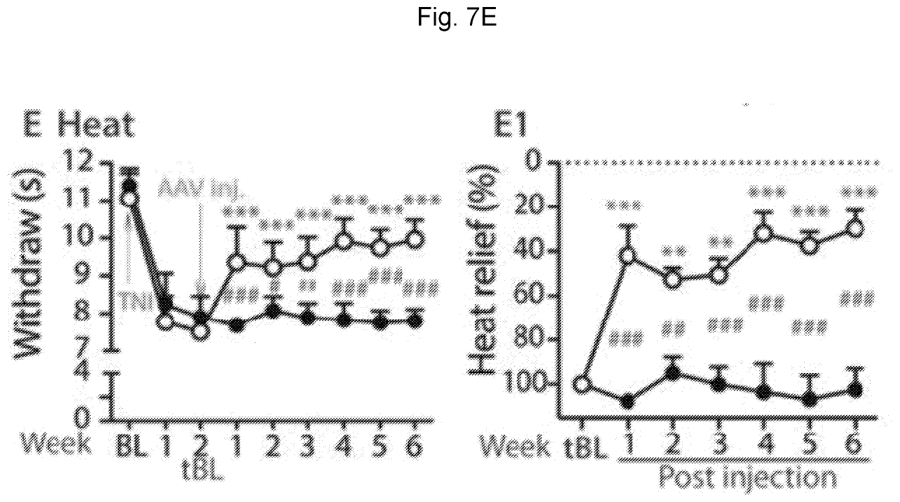

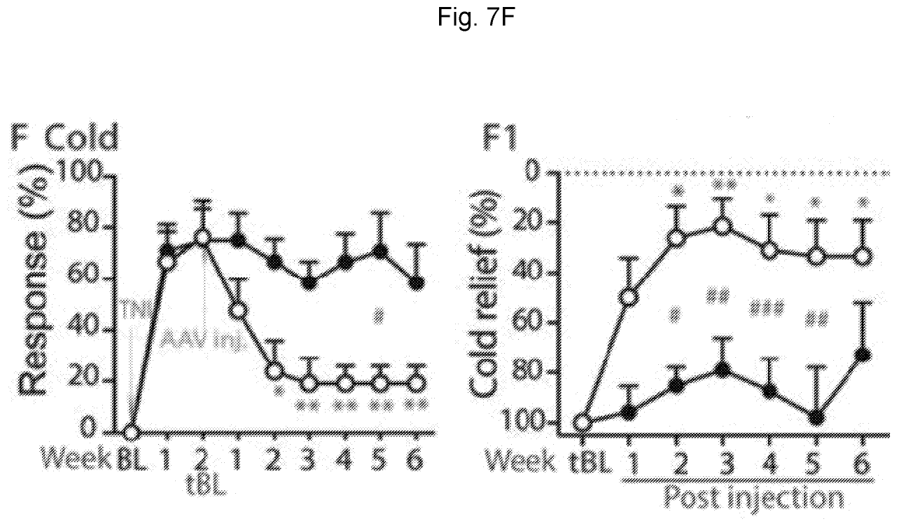

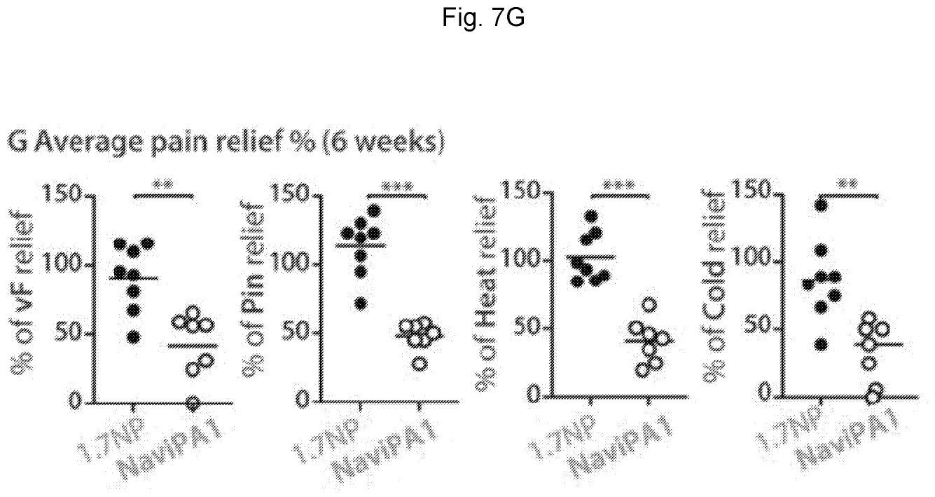

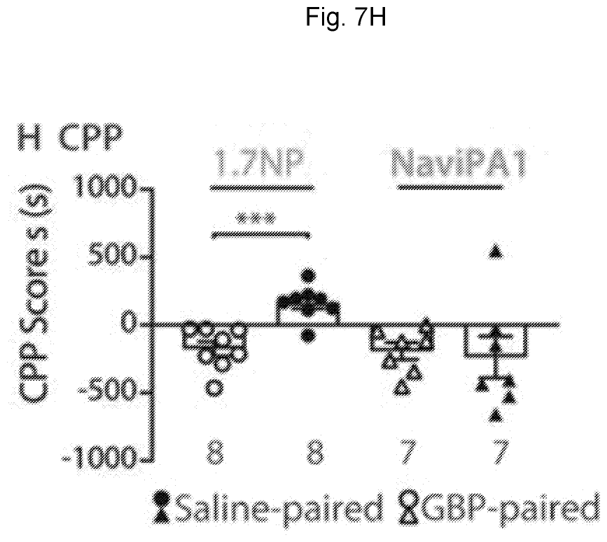

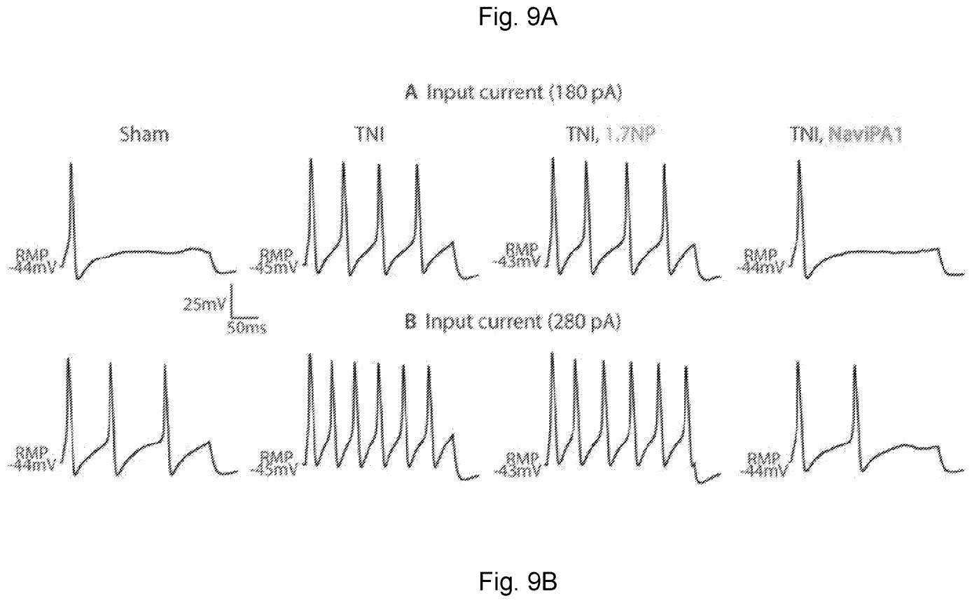

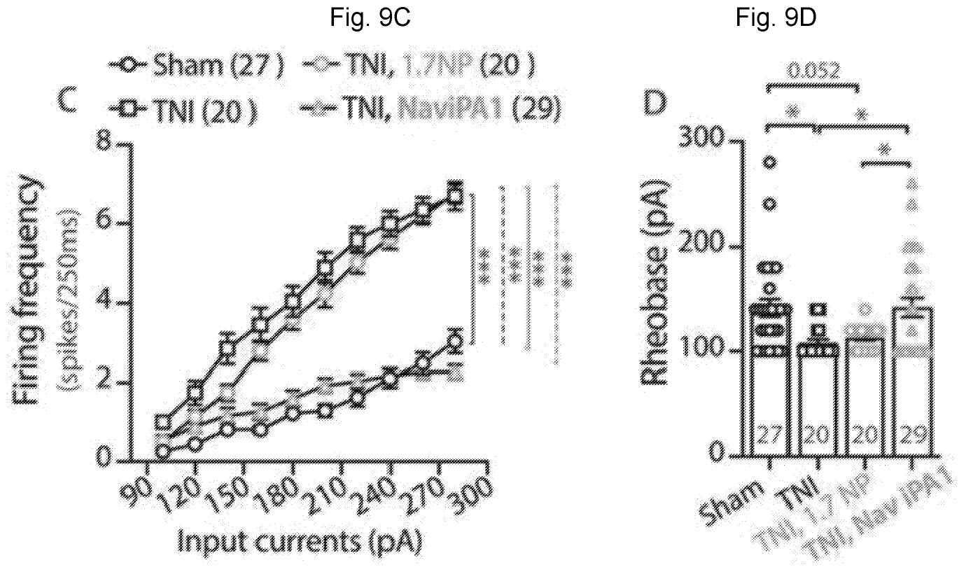

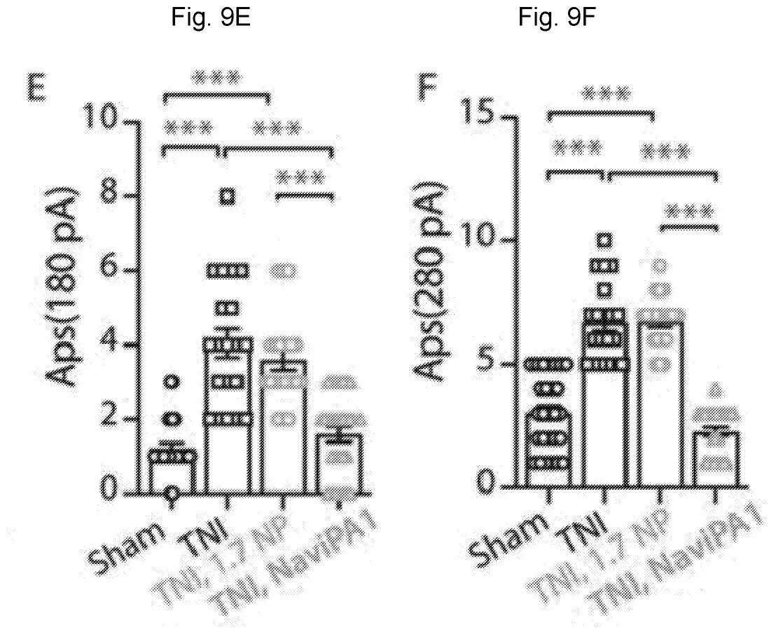

The tetrodotoxin sensitive (TTXs) voltage gated sodium channel Naᵥ1.7, which is encoded by the SCN9A gene in humans, is expressed on nociceptive neurons. The inventors designed novel peptide aptamer inhibitors of Naᵥ1.7 that bind to intrinsically disordered regions (IDRs) of Naᵥ1.7 (FIG. 1). Furthermore, the inventors developed a novel treatment for pain comprising administering infectious particles, e.g., adeno-associated viruses, encoding the disclosed peptide aptamers. The inventors discovered that administration of the infectious particles was effective in reducing allodynia and pain hypersensitivity in animals subsequent to tibial nerve injury (TNI), a rat model of neuropathic pain. See, FIGS. 7 and 9.

### Polypeptide Aptamers

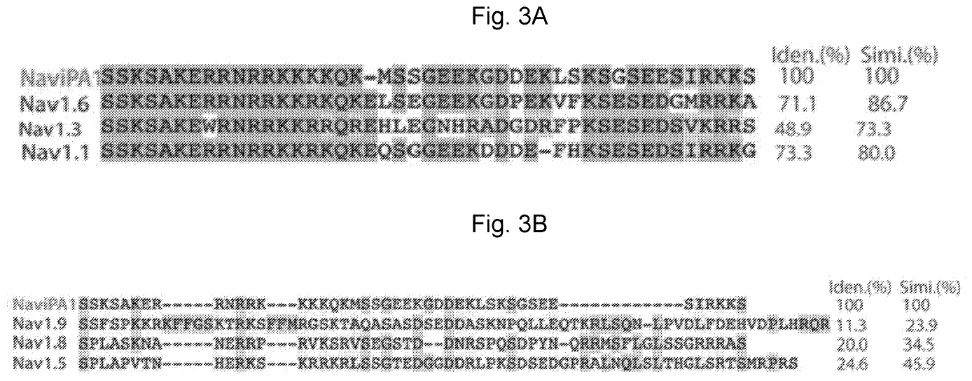

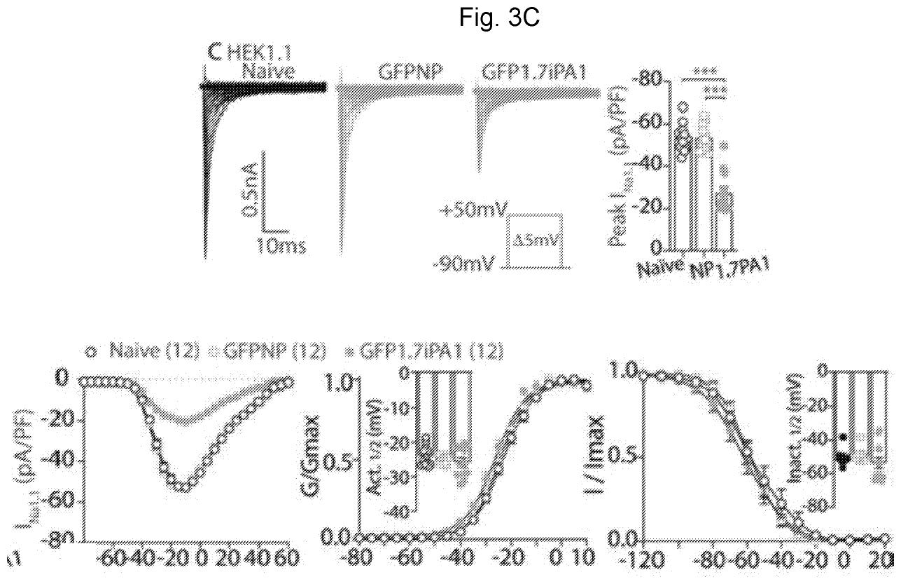

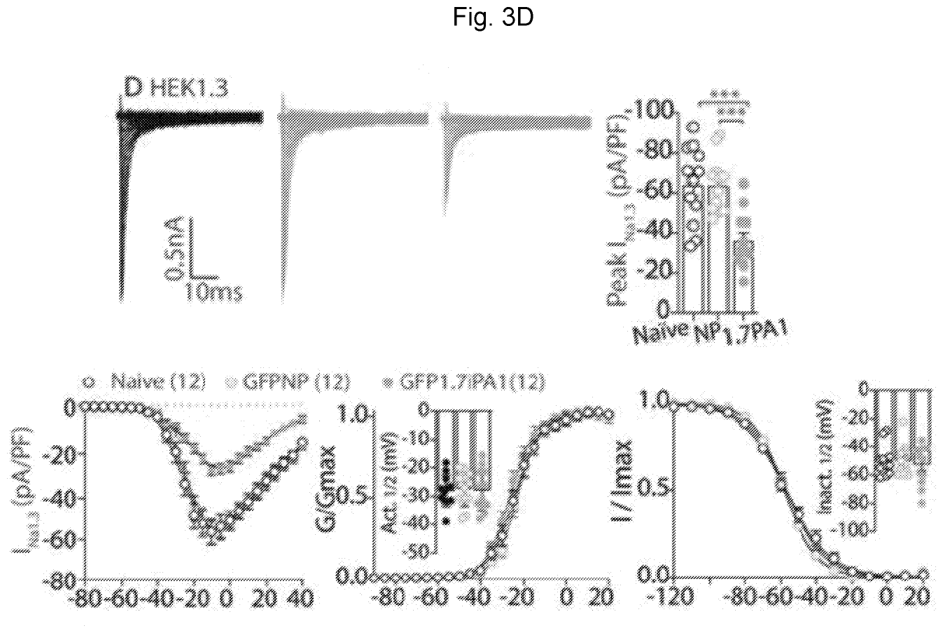

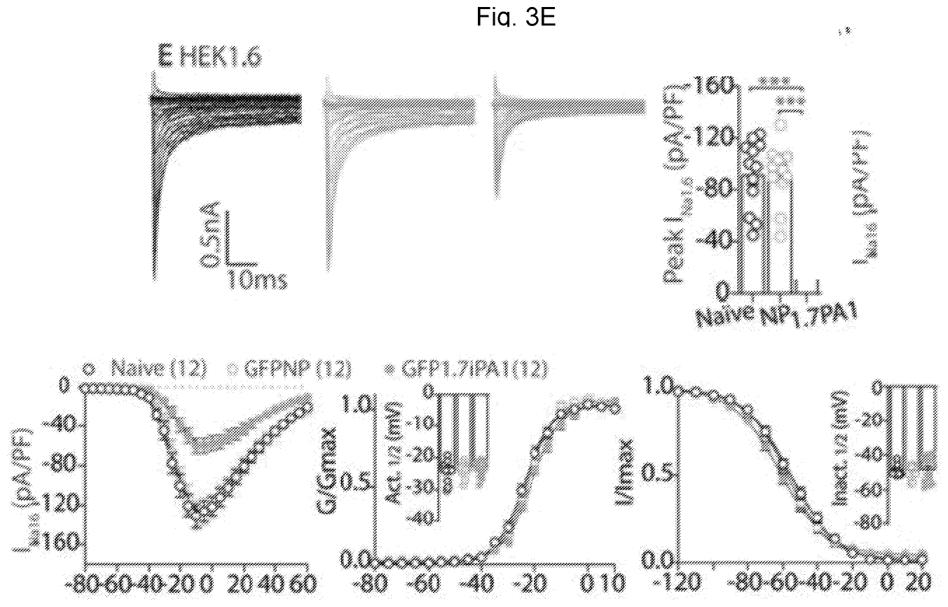

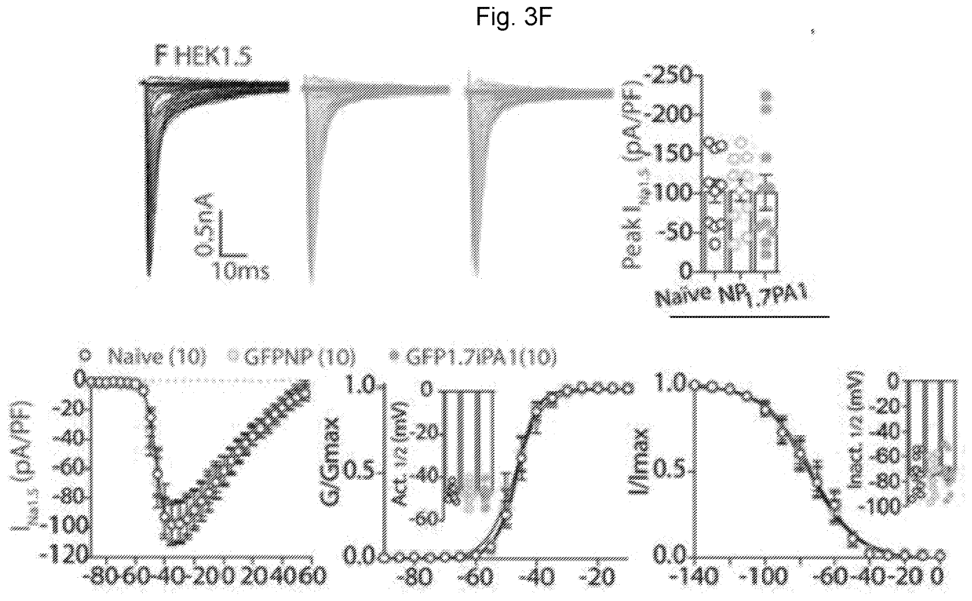

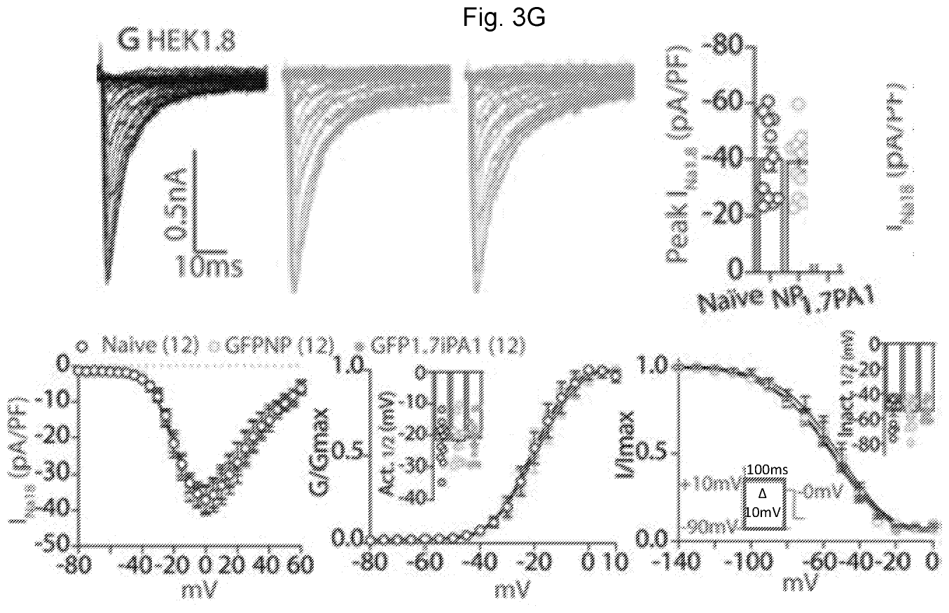

In an aspect of the current disclosure, polypeptide aptamers comprising a sequence are provided. As discussed above, the inventors designed the peptide aptamers SEQ ID NOs: 1-9 (corresponding to peptides iPA1, iPA2, iPA3, iPA4, iPA5, iPA6, iPA7, iPA8, and iPA9 of FIG. 1E) to bind to IDRs of Nav1.7. Further, the inventors discovered that SEQ ID NO: 1 inhibits I_{Na1.7}, I_{Na1.6}, and I_{Na1.3}, but not the tetrodotoxin resistant (TTXr) voltage-gated sodium channels I_{Na1.5} and I_{Na1.8}. See, e.g., FIGS. 3C-3G and Table 2. It is believed that the ability of the disclosed polypeptide aptamers to inhibit multiple Naᵥ isoforms present on nociceptive neurons may provide a therapeutic advantage through a multi-pronged reduction in sensitivity. Accordingly, the inventors believe that the disclosed polypeptide aptamers may possess an advantage over more specific Naᵥ1.7-targeted approaches to achieve analgesia.

As used herein, “aptamer” or “peptide aptamer” refers to a short peptide, e.g., less than 100 amino acids in length. Peptide aptamers are designed to bind to a target molecule, e.g., a target protein, e.g., Naᵥ1.7. Though peptide aptamers may comprise a portion of a full-length protein, peptide aptamers do not include the full length protein from which they may be derived.

Surprisingly, the inventors also discovered that iPA2 (SEQ ID NO: 2) increased the current density of Naᵥ1.7 (FIG. 1J).

The disclosed polypeptide aptamers may comprise a sequence selected from the group consisting of SEQ ID NOs: 1-9, 50, and 52, or a sequence with at least 85%, at least 86%, at least 87% at least 88%, at least 89%, at least 90%, at least 91%, at least 92% at least 93%, at least 94%, at least 95%, at least 96%, at least 97%, at least 98%, or at least 99% identity to one of SEQ ID NOs: 1-9, 50, and 52. The sequence may be selected from SEQ ID NOs: 1, 4, or 6, or a sequence with at least 85%, at least 86%, at least 87% at least 88%, at least 89%, at least 90%, at least 91%, at least 92% at least 93%, at least 94%, at least 95%, at least 96%, at least 97%, at least 98%, or at least 99% identity to one of SEQ ID NOs: 1, 4, or 6. The sequence may be SEQ ID NO: 1 or a sequence with at least 85%, at least 86%, at least 87% at least 88%, at least 89%, at least 90%, at least 91%, at least 92% at least 93%, at least 94%, at least 95%, at least 96%, at least 97%, at least 98%, or at least 99% identity to SEQ ID NO: 1.

The disclosed polypeptide aptamers may consist essentially of a sequence selected from SEQ ID NOs: 1-9, or a sequence with at least 85%, at least 86%, at least 87% at least 88%, at least 89%, at least 90%, at least 91%, at least 92% at least 93%, at least 94%, at least 95%, at least 96%, at least 97%, at least 98%, or at least 99% identity to one of SEQ ID NOs: 1-9, 50, and 52. However, it is to be understood that the disclosed polypeptide aptamers may comprise additional moieties, e.g., a detectable marker, that do not interfere with the ability of the polypeptide aptamers to bind to and/or modify the function of TTS Navs.

The disclosed polypeptide aptamers may consist of a sequence selected from SEQ ID NOs: 1-9, or a sequence with at least 85%, at least 86%, at least 87% at least 88%, at least 89%, at least 90%, at least 91%, at least 92% at least 93%, at least 94%, at least 95%, at least 96%, at least 97%, at least 98%, or at least 99% identity to one of SEQ ID NOs: 1-9, 50, and 52.

As used herein, “polypeptide” refers to a polymer of amino acids joined together by peptide bonds.

The term “sequence identity” as used herein refers to the extent that sequences are identical on a nucleotide-by-nucleotide basis or an amino acid-by-amino acid basis over a window of comparison. Nucleic acid and protein sequence identities can be evaluated by using any method known in the art. For example, the identities can be evaluated by using the Basic Local Alignment Search Tool (“BLAST”). The BLAST algorithms determine homology of sequences by identifying similar segments between a query amino or nucleic acid sequence and a test sequence which is preferably obtained from protein or nuclei acid sequence database. The BLAST program can be used with the default parameters or with modified parameters provided by the user.

The term “percentage of sequence identity” is calculated by comparing two optimally aligned sequences over the window of comparison, determining the number of positions at which the identical nucleic acid base (e.g., A, T, C, G) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

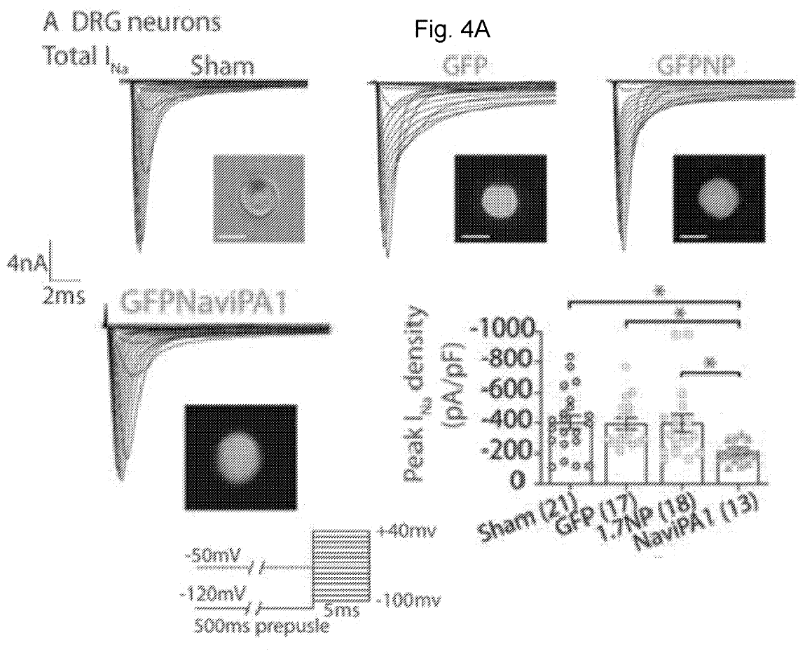

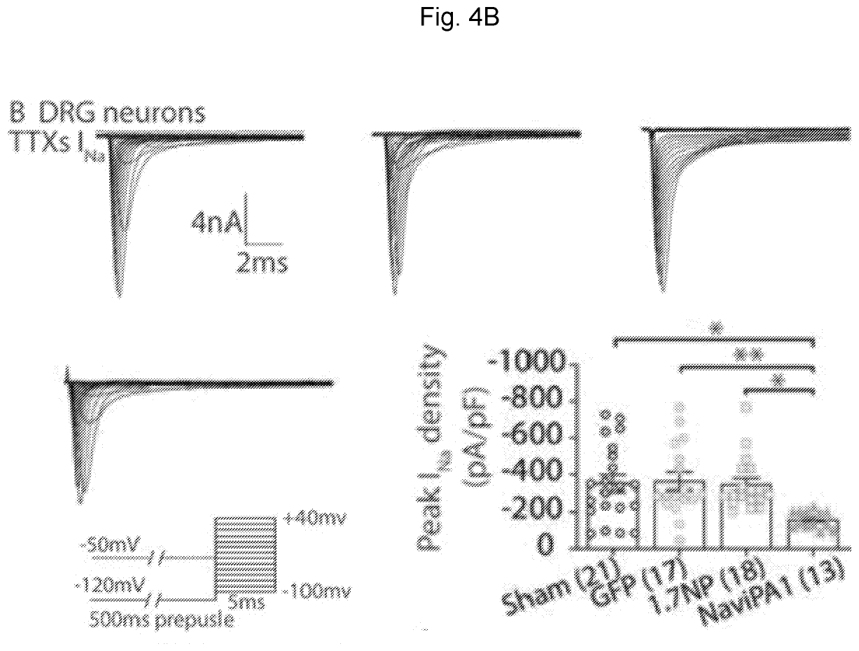

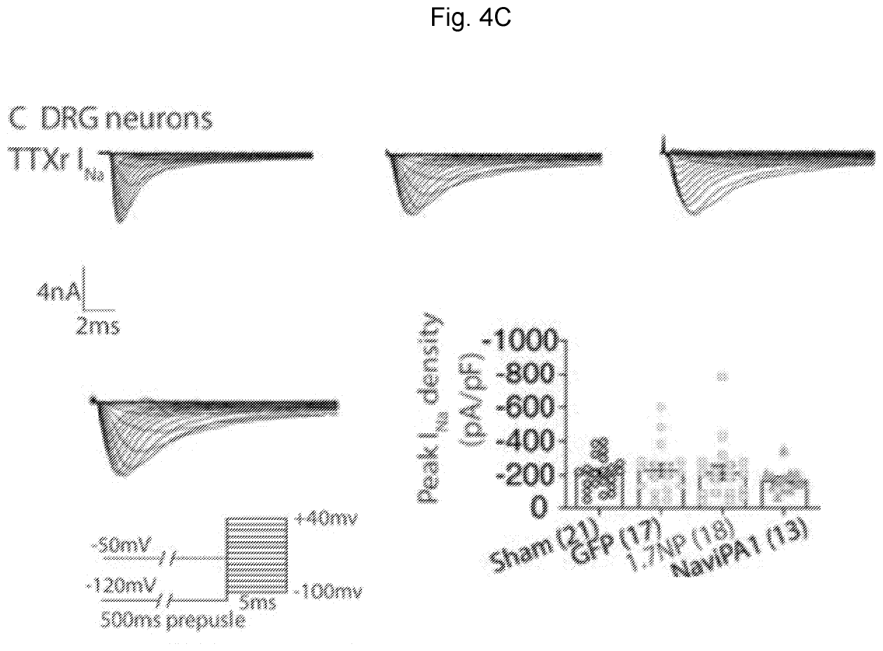

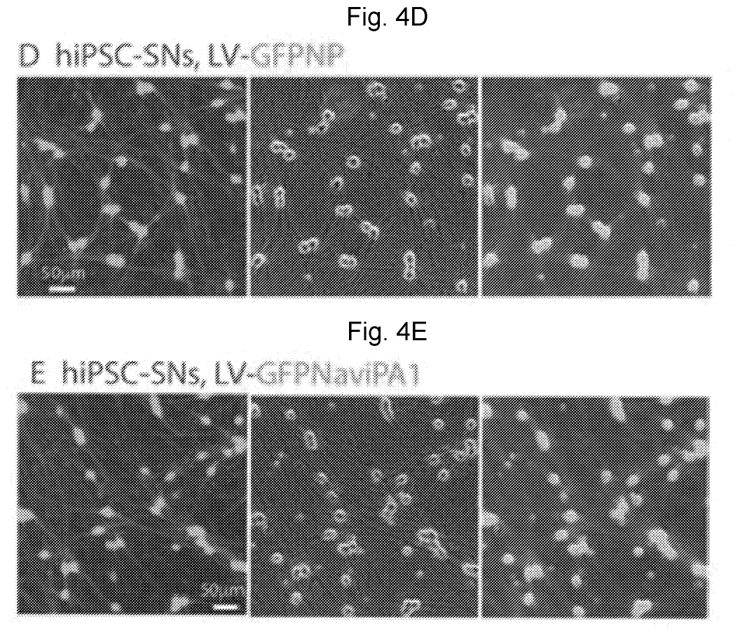

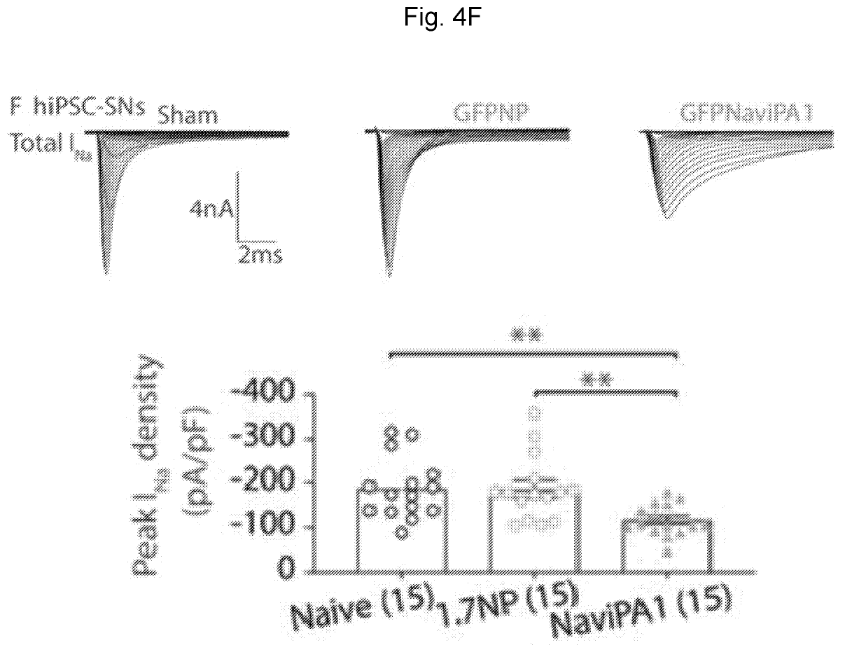

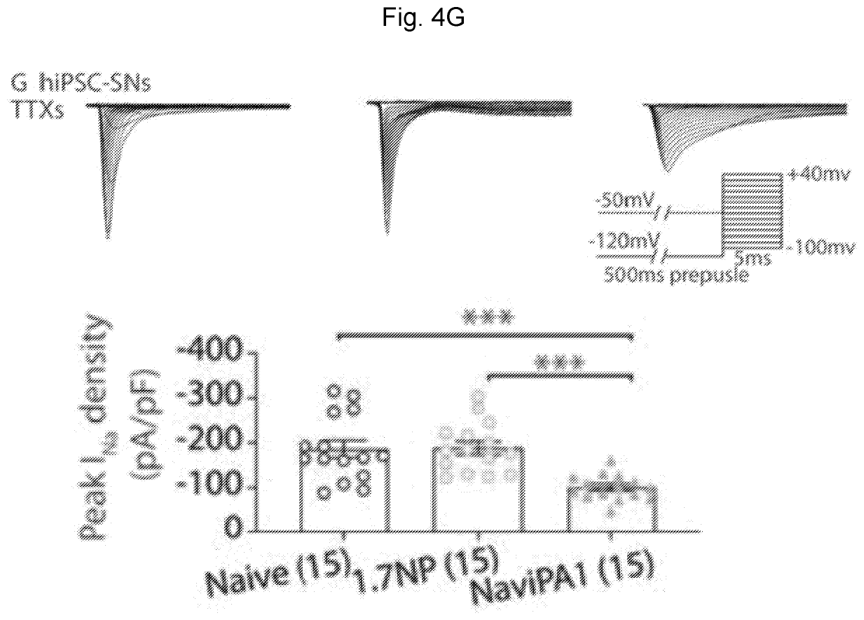

The inventors have demonstrated that the disclosed aptamers functionally interact with, e.g., Naᵥ1.7 when present as isolated aptamer sequences, e.g., consisting only of one of SEQ ID NOs: 1-9 (FIG. 1J), as well as when linked to a detectable marker, e.g., green fluorescent protein (GFP) (FIG. 4A). Thus, though the disclosed polypeptide aptamers may comprise only a sequence selected from SEQ ID NOs: 1-9, 50, and 52, or a sequence with at least 85% identity to one of SEQ ID NOs: 1-9, 50, and 52, they may further comprise additional moieties and retain the capacity to interact, e.g., inhibit the function of a voltage gated sodium channel, e.g., Naᵥ1.7.

As used herein, “detectable marker” refers a marker that is readily identifiable by a particular property of the detectable marker, e.g., electrochemiluminescence, chemiluminescence, fluorescence, radioactivity, nucleotide sequence or amino acid sequence, etc. In some embodiments, the detectable marker is a fluorescent protein, e.g., green fluorescent protein (GFP), e.g., SEQ ID NO: 23.

The disclosed polypeptide aptamers may comprise a linker, e.g., SEQ ID NO: 10. The linker may be attached, e.g., covalently attached, e.g., by peptide bond, to the disclosed polypeptide aptamers. The disclosed polypeptide aptamers may comprise: a detectable marker, e.g., GFP, a linker, and a sequence, as configured from N-terminus to C-terminus. However, it is to be understood that the disclosed polypeptide aptamers are not limited to any such configuration and a detectable marker or other moiety may be located at the N-terminus, C-terminus, or other location on the disclosed polypeptide aptamers.

The disclosed polypeptide aptamers may further comprise a “purification tag” which, as used herein, refers to a moiety that binds to a substrate, e.g., column, beads, coated surface, etc. Exemplary purification tags comprise, without limitation, streptavidin tags, biotin, FLAG tags, and the like.

### Polynucleotides

In another aspect of the current disclosure, polynucleotides comprising a nucleotide sequence encoding the disclosed polypeptide aptamers are provided.

The disclosed polynucleotides may further comprise a regulatory region, e.g., a promoter, enhancer, etc., that is operably linked to the nucleotide sequence encoding the disclosed polypeptide aptamers. A suitable promoter may be, e.g., a hybrid human cytomegalovirus (CMV) enhancer/chicken β-actin (CBA) promoter.

As used herein, a polynucleotide is “operably linked” or “operably connected” when it is placed into a functional relationship with a second polynucleotide sequence.

It is envisioned that the disclosed polynucleotides may be utilized to express the disclosed polypeptide aptamers in cells, e.g., human cells, or in a human subject. Accordingly, the disclosed polynucleotides may be, e.g., a plasmid, a minicircle, and may comprise regulatory regions allowing for viral delivery of the polynucleotides.

For example, the disclosed polynucleotides may comprise viral regulatory regions, e.g., adeno-associated virus (AAV) inverted terminal repeat sequences (ITR).

As used herein, the term “inverted terminal repeat” (ITR) sequences refers to sequences of DNA that flank the portion of the AAV genome that allows insertion of the genome into the host cell. ITRs are the only cis-acting element required for AAV formation. Therefore, the additional elements of the AAV genome may be added in trans to facilitate formation of a complete viral particle. Accordingly, the only portion of the viral genome that is required to be included in the DNA carried by the AAV are the 5′ and 3′ ITRs. Suitable ITRs are known in the art and may include, for example, 5′ ITR with the sequence SEQ ID NO: 28 and the 3′ ITR with the sequence SEQ ID NO: 29.

### Infectious Particles

The inventors have demonstrated that delivery of the disclosed polypeptide aptamers mediated by infectious particles, i.e., an adeno-associated viral vector, is an effective strategy to induce local anesthesia in nociceptive neurons. See, e.g., FIGS. 7 and 9. Therefore, in another aspect of the current disclosure, infectious particles comprising the disclosed polynucleotides encoding the polypeptide aptamers of the instant disclosure are provided.

Suitable infectious particles, also referred to herein as “viral vectors” are known and commercially available in the art. For example, see Deverman et al. (Cre-dependent selection yields AAV variants for widespread gene transfer to the adult brain, Nature Biotechnology, 34 (2): 204-209, 2016) and Chan et al. (Engineered AAVs for efficient noninvasive gene delivery to the central and peripheral nervous system, Nature Neuroscience, 20 (8): 1172-1179, 2017), which are incorporated herein by reference in their entirety. A skilled artisan will be familiar with the elements and configurations necessary for vector construction to encode the constructs described herein. Exemplary infectious particles comprise AAV particles, adenovirus particles, herpesvirus particles, baculovirus particles, or any other suitable virus particles.

The disclosed infectious particles may comprise adeno-associated viruses (AAVs) comprising the aforementioned viral regulatory regions which direct expression of a product from the disclosed polynucleotides, which are contained within the virus or associated with the virus, in a host cell, where the expression product of the polynucleotides comprises one or more of the disclosed polypeptide aptamers. The expression product of the polynucleotides can comprise a sequence, e.g., SEQ ID NO: 1, 2, 3, 4, 5, 6, 7, 8, or 9, or another polypeptide aptamer contemplated in the instant disclosure. In some cases, virus is selected from AAV type 1, AAV type 2, AAV type 3 (including types 3A and 3B), AAV type 4, AAV type 5, AAV type 6, AAV type 7, AAV type 8, AAV type 9, AAV type 10, AAV type 11, among others. In exemplary embodiments, the AAV is AAV type 6. See, e.g., FIGS. 7 and 9.

### Pharmaceutical Compositions

It is envisioned that the disclosed polypeptide aptamers may be administered to a subject. Therefore, in another aspect of the current disclosure, pharmaceutical compositions comprising the disclosed polypeptide aptamers and a pharmaceutically acceptable carrier or excipient.

As used herein, “an effective amount” refers to the amount or dose of the disclosed compositions, upon single or multiple dose administration to a subject, which provides the desired effect in the subject under diagnosis or treatment.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical dose may contain from about 0.01 mg/kg to about 100 mg/kg (such as from about 0.05 mg/kg to about 50 mg/kg and/or from about 0.1 mg/kg to about 25 mg/kg) of the disclosed polypeptide aptamers.

A typical dose of the disclosed pharmaceutical compositions comprising viral particles, may comprise an amount from about 5 μg of viral DNA to about 100 μg viral DNA, about 10 μg viral DNA to about 50 μg viral DNA. The dose of the disclosed viral particles administered to a subject may comprise about 1×10⁸ viral particles to about 1×10¹² viral particles. The dose may comprise 1×10¹⁰ viral particles.

Compositions comprising the disclosed polypeptide aptamers can be formulated in a unit dosage form, each dosage containing from about 1 to about 500 mg of each polypeptide aptamer individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Direct injection into the dorsal root ganglion is an illustrative route of administering the compounds employed in the compositions and methods disclosed herein. Other illustrative routes of administration include transdermal, percutaneous, intravenous, intramuscular, intranasal, buccal, intrathecal, intracerebral, oral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, inhalation, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods

The inventors discovered that contacting cells, e.g., neurons, with the one or more of the disclosed polypeptide aptamers is effective in reducing excitation of the neurons due to depolarization by the Naᵥ1.7 channel. See, e.g., FIG. 1J. The disclosed polypeptide aptamers may be contacted to cells by, e.g., direct contact of the polypeptide aptamers to the cell, expression of polynucleotides encoding the disclosed polypeptide aptamers in the cell, or in another cell from which the polypeptide aptamers are secreted, or by contacting the cell with an infectious particle, e.g., an AAV comprising the disclosed polynucleotides which are configured to express in the cell.

Therefore, in another aspect of the current disclosure, methods are provided. In some embodiments, the methods comprise contacting a cell with the disclosed polypeptide aptamers, the disclosed polynucleotides, or the disclosed infectious particles or cells comprising the disclosed polynucleotides.

As used herein, “contacting” refers to contacting a cell directly or indirectly in vitro, ex vivo, or in vivo (e.g., within a subject as defined herein). Contacting a cell may include addition of a disclosed composition, e.g., the disclosed polypeptide aptamers, polynucleotides, or infectious particles, or pharmaceutical compositions, to a sample comprising a cell, tissue, etc., or administration of the composition to a subject. Contacting may include administration of the composition to a solution, a cell, a tissue, a mammal, a subject, a patient, or a human. For example, contacting a cell with a composition may include adding the composition to a cell culture.

The disclosed methods may comprise methods of reducing or inhibiting stimulation of a neuron comprising contacting a cell with the disclosed polypeptide aptamers, the disclosed polynucleotides, or the disclosed infectious particles comprising the disclosed polynucleotides.

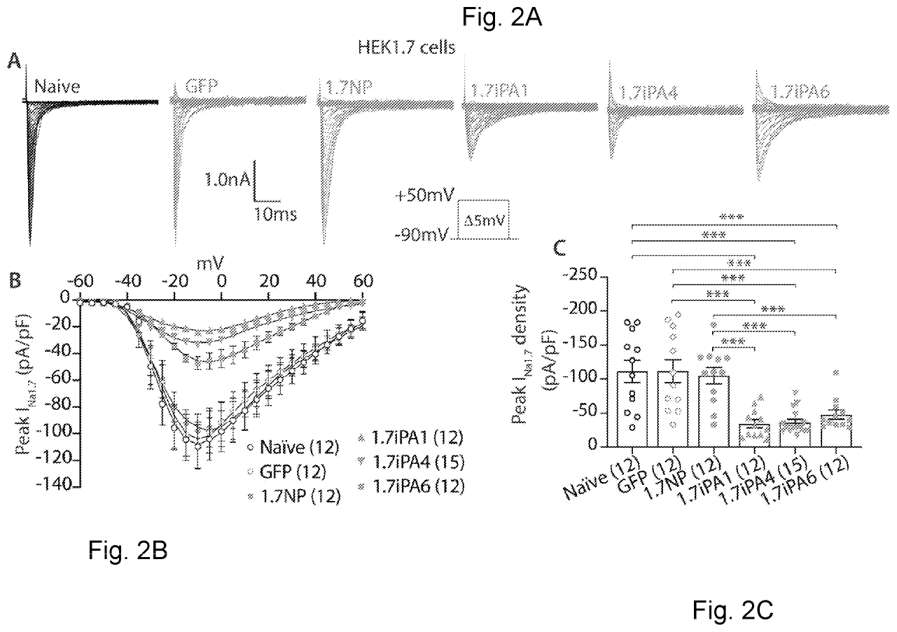

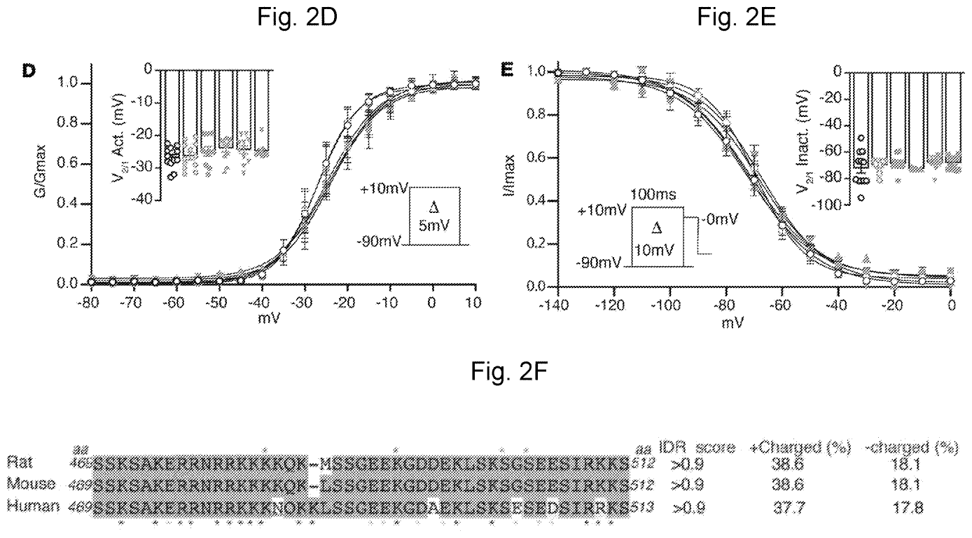

The inventors demonstrated that contacting neurons with the disclosed polypeptide aptamers, e.g., polypeptides comprising sequences SEQ ID NOs: 1, 4, or 6, reduces peak Naᵥ1.7 current density in the neuron by at least 50% as measured by patch clamp electrophysiology (FIG. 2A). Further, the inventors demonstrated that the methods do not change channel steady-state inactivation properties of Naᵥ1.7 channels in the neuron (FIGS. 2B-F).

The inventors demonstrated that administering AAVs with viral genomes encoding the disclosed polypeptide aptamers was effective in reducing allodynia and sensitivity in animals subjected to tibial nerve injury, a model of neuropathic pain. See, FIGS. 7 and 9. Therefore, in other aspects of the disclosure, the methods comprise administering the disclosed pharmaceutical compositions comprising infectious particles to a subject.

Furthermore, the disclosed methods include methods of treating neuropathic pain in a subject in need thereof comprising administering a therapeutically effective amount of the disclosed pharmaceutical compositions to a subject in need thereof to treat the neuropathic pain in the subject.

The disclosed methods may further comprise methods of treating pain due to a traumatic nerve injury in a subject in need thereof, the method comprising administering a therapeutically effective amount of the disclosed pharmaceutical compositions to the subject to treat the pain due to the traumatic nerve injury in the subject.

The inventors have shown that direct administration of the disclosed pharmaceutical compositions to the dorsal root ganglion (DRG) of animals is effective in treating neuropathic pain in the animals. Therefore, administration, in the context of the disclosed methods, may comprise delivery to a dorsal root ganglion in the subject.

In some cases, the disclosed methods inhibit nociceptor excitation. Nociceptors are specialized sensory neurons (e.g., nociceptive dorsal root ganglion neurons) that have A8- and C-fibers in the peripheral nerve and sensory non-corpuscular “free nerve endings” in innervated organs. Nociceptors transduce mechanical, thermal, and chemical stimuli into a depolarizing sensor potential. If the depolarization is sufficiently large, it opens voltage-gated ion channels and triggers the generation of action potentials that are conducted to the dorsal horn of the spinal cord or the brainstem.

Targeted injection of the disclosed compositions limits systemic exposure upon administration to the subject. For example, targeted delivery can comprise injecting an infectious particle comprising a polynucleotide encoding one or more of the disclosed polypeptide aptamers into or nearby neural tissue of the subject. Examples of neural tissues into which a composition described herein can be injected include, without limitation, ganglia (e.g., the dorsal root ganglia), spinal nerve, preganglionic fibers, and paraganglia. Examples of neural tissues nearby which a composition described herein can be injected include, without limitation, the periganglionic subarachnoid space.

The disclosed polynucleotides may be administered using any appropriate delivery vehicle. For example, in some cases, a nucleic acid encoding a polypeptide aptamer useful for treating pain may be incorporated into a delivery vehicle that can drive expression of the nucleic acid. Examples of delivery vehicles include, without limitation, non-viral vectors (e.g., plasmids (e.g., expression plasmids), liposomes, and polymersomes) and viral vectors (e.g., adeno-associated virus vectors, HSV vectors, and lentiviral vectors). For example, the disclosed polynucleotides may be delivered using an adeno-associated virus (AAV) vector. As used herein, the term “adeno-associated virus” (AAV) includes, without limitation, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. The genomic sequences of various AAV and autonomous parvoviruses, as well as the sequences of the ITRs, Rep proteins, and capsid subunits are known in the art. Such sequences may be found in the literature or in public databases such as the GenBank database.

In some cases, the AAV vector is produced with modified capsids, which have been shown to improve gene transfer efficiency with potentially reduced immunogenicity compared with naturally occurring serotypes. See, e.g., Buning & Srivastava, Mol Ther Methods Clin Dev 12:248-265, (2019). For example, Anc80L65 (anc80), which is a novel AAV capsid designed from in silico reconstruction of the viral evolutionary lineage, has been demonstrated robust transduction capabilities after local delivery in various tissues. See Wang et al., PLoS One 12: e0182473, (2017); Hudry et al., Mol Ther Methods Clin Dev 10:197-209, (2018).

### Cells

In another aspect of the current disclosure, cells comprising the polypeptide aptamers, polynucleotides, or infectious particles of the instant disclosure are provided. The cells may be mammalian cells, e.g., human cells. The cells may be cultured cells, e.g., human embryonic kidney (HEK) cells or induced pluripotent stem cells (iPSCs), or primary neurons, either isolated from a subject or in vivo.

### Cells Expressing Naᵥ1.8

The inventors generated cells comprising a heterologous polynucleotide encoding human Naᵥ1.8 protein which express functional human Naᵥ1.8 protein and which are suitable for electrophysiological experiments, also referred to herein as “HEK1.8 cells”. See, e.g., FIGS. 3G and 11.

As used herein, “heterologous polynucleotide” refers to a polynucleotide that is not normally present in the cells and has been introduced to the cells by, e.g., transduction, transfection, lipofection, nucleofection, electroporation, or other means known in the art. Heterologous polynucleotides may be, e.g., plasmids or other expression vectors.

The Naᵥ1.8 protein may have the sequence SEQ ID NO: 20 and may be encoded by a heterologous polynucleotide comprising SEQ ID NO: 21.

The heterologous polynucleotide may comprise sequences encoding Naᵥ1.8a (amino acid sequence SEQ ID NO: 20) and Navb2 (amino acid sequence SEQ ID NO: 24) from a single open reading frame (ORF) linked by a sequence encoding a 2A self-processing sequence derived from porcine teschovirus-1 (P2A), e.g., SEQ ID NO: 26, and a furin cleavage site, e.g., R,X,X,R or R,X,K/R,R, wherein X is any amino acid. The furin cleavage site may be RKRR (SEQ ID NO: 27). The polynucleotide may comprise the nucleotide sequences SEQ ID NOs: 21 and 25, which encode Naᵥ1.8a and Navb2, respectively. The polynucleotide may comprise SEQ ID NO: 22, i.e., pcDNA3.1-SCN10A-Furin-P2A-SCN2B.

The cells may comprise human cells, e.g., human embryonic kidney (HEK) cells.

### Methods of Testing a Compound or Agent for the Ability to Modulate Voltage Gated Sodium Channel Function

It is further contemplated that the disclosed polypeptide aptamers, polynucleotides, infectious particles, cells, or the cells expressing Naᵥ1.8 may be used in methods of testing a compound or agent for the ability to modulate voltage gated sodium channel function.

For example, one of skill in the art may use the disclosed polypeptide aptamers to modulate the function of a voltage gated sodium channel, e.g., Naᵥ1.7, as a positive control for inhibition of Naᵥ1.7 function. Thus, methods of testing a compound or agent may comprise contacting a compound or agent to a cell, contacting at least one of the disclosed compositions, e.g., the disclosed polypeptide aptamers, polynucleotides, or infectious particles, to a cell, wherein if a measured parameter in the cell, e.g., an electrophysiological parameter, e.g., current density, is altered to similarly, i.e., demonstrates an effect within, e.g., 5%, 10%, 20%, 30%, of the effect induced by the disclosed composition, then the compound or agent is a modulator of voltage gated sodium channel function.

The disclosed cells expressing Naᵥ1.8 may be used in methods of determining if a compound or agent is a modulator of Naᵥ1.8 comprising contacting a proposed Naᵥ1.8 modulating compound or agent to the cells expressing Naᵥ1.8 and measuring a parameter related to Naᵥ1.8 function, e.g., an electrophysiological parameter, e.g., current density, wherein if the compound or agent modulates the parameter related to Naᵥ1.8 function then the compound or agent is a modulator of Naᵥ1.8 function.

### Systems, Kits, and Platforms

In another aspect of the current disclosure, systems, kits, and platforms are provided. The systems, kits, and platforms may comprise the disclosed polypeptide aptamers, polynucleotides, infectious particles, or cells. The systems, kits, and platforms may further comprise reagents for performing electrophysiological experiments. The systems, kits, and platforms may also comprise instructions for use.

The disclosed systems, kits, and platforms may comprise the disclosed cells expressing human Naᵥ1.8 protein. It is envisioned that such cells may be used as in studies or in evaluation of new agents targeting the Naᵥ1.8 protein.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter.

### Example 1—Peripheral-Targeted Analgesia Via AAV-Mediated Sensory Neuron-Specific Inhibition of Multiple Pronociceptive Sodium Channels in Rat

This study reports that targeting intrinsically disordered regions (IDRs) of Naᵥ1.7 protein facilitated discovery of sodium channel inhibitory peptide aptamers (NaviPA) for virus (AAV)-mediated, sensory neuron-specific analgesia. A adeno-associated multipronged inhibition of I_{Na1.7}, I_{Na1.6}, I_{Na1.3}, and I_{Na1.1}. but not I_{Na1.5} and I_{Na1.8} was found for a prototype, named NaviPA1, which was derived from the Naᵥ1.7 intracellular loop 1 and is conserved among the TTXs Nav subtypes. NaviPA1 expression in primary sensory neurons (PSNs) of dorsal root ganglia (DRG) produced significant inhibition of TTXs I_{Na} but not TTXr I_{Na}. DRG injection of AAV6-encoded NaviPA1 significantly attenuated evoked and spontaneous pain behaviors in both male and female rats with neuropathic pain induced by tibial nerve injury (TNI). Whole-cell current clamp of the PSNs showed that NaviPA1 expression normalized PSN excitability in TNI rats, suggesting that NaviPA1 attenuated pain by reversal of injury-induced neuronal hypersensitivity.

Immunohistochemistry revealed efficient NaviPA1 expression restricted in PSNs and their central and peripheral terminals, indicating PSN-restricted AAV biodistribution. Inhibition of sodium channels by NaviPA1 was replicated in the human iPSC-derived sensory neurons. These results summate that NaviPA1 is a promising analgesic lead that, combined with AAV-mediated PSN-specific block of multiple TTXs Navs, has potential as peripheral nerve-restricted analgesic therapeutics.

### INTRODUCTION

Voltage-gated sodium channels (Navs) are key regulators of neuronal excitability and pain sensations (1). Mammals possess nine isoforms of Navs, of which Naᵥ1.7, Naᵥ1.8, and Nav1.9 are preferentially expressed in the primary sensory neurons (PSNs) of dorsal root ganglia (DRG) (2). The prominent roles of these Nav isoforms in human pain have been validated (2). Nav1.6, Nav1.1, and Nav1.3 are also expressed in PSNs and have been reported as possible targets for analgesics (3, 4). Currently, Nav1.7 is the leading target among Navs for developing analgesic therapies (5).

Numerous efforts have been made over the last decades to develop selective and effective Nav1.7 blockers to treat pain in clinic (6), but the success has been limited. Most of the available small-molecule Nav1.7 blockers tested to treat pain are insufficient in target engagement, lack of targeting specificity or selective bioavailability in pain axis, and their global distribution contributes to cardio-toxicity, motor impairments, and CNS side-effects (6). Development of biologics targeting Nav1.7 is an alternative growing-trend (7, 8) for analgesia. Nav1.7 neutralizing monoclonal antibodies have analgesic efficacy, but the results are inconsistent (6). Tarantula peptide Nav1.7 blockers are effective analgesics but have poor membrane permeability, inadequate Nav1.7 selectivity, and short half-lives (6). Nav1.7-RNAi (6) and CRISPR-dCas9 or ZEN epigenetic Nav1.7 suppression for analgesic gene therapy have been proposed (9), but these interventions at mRNA and epigenetic levels have a concern of lacking the specificity of direct channel intervention, reducing safety and permitting off-target effects (6, 10), and anti-Cas9 immunity creates additional challenge for CRISPR gene therapies (11).

Small peptides derived from pronociceptive ion channels as functionally interfering peptide aptamers (iPA) are highly effective and selective, allowing block of specific nociceptive signaling (12, 13). Intrinsically disordered regions (IDRs) of ion channel proteins are commonly engaged in promiscuous interactomes, which are important players in multiple signaling regulations and are recognized as new and promising drug targets (14). We speculated that Nav1.7-IDRs contain short functional IDR domains that could play critical roles in modulating Nav1.7 functions and can be developed as Nav1.7iPAs (1.7iPA). Furthermore, the substantial conservation of Nav subtype sequences implies that a given 1.7iPA could interact with other Nav subtypes that have homologous sequences to Nav1.7 and thereby enable multipronged engagement of Nav subtypes. Because multiple PSN-Navs contribute to nociceptive electrogenesis and pain pathogenesis, it is conceivable that AAV-mediated expression of such multipronged NaviPA restricted in DRG-PSNs to inhibit several pronociceptive Navs could be an analgesic advantage compared to block of only a single Nav subtype (15-17).

We here describe a novel strategy by which highly selective and nontoxic NaviPAs were designed and developed from Navs-IDRs. A prototypic NaviPA1 derived from Nav1.7 intracellular loop 1 and conserved in TTXs Nav subtypes showed multipronged inhibition of Nav1.7, Nav1.6, and Nav1.3, and Nav1.1 channels. NaviPA1 expression in rat PSNs rendered significant TTXs but not TTXr I_{Na} inhibition. AAV-mediated NaviPA1 expression selectively in the PSNs responsible for pain pathology in rat model produced efficient analgesia while avoiding off-site biodistribution that causes side-effects. Together, these results indicate that AAV-mediated PSN-specific, combined block of multiple nociceptive Navs, has potential for future therapeutic development.

### Results

In Silico Design of 1.7iPAs from Nav1.7-IDRs

The candidate iPAs were designed through a priori strategy aimed to define the short linear functional disordered peptides from the intrinsically disordered domains (IDDs) (12), initially from Nav1.7 protein IDRs, on the hypothesis that Nav1.7 IDDs contain the functional sequences that modulate Nav1.7 channel function. We analyzed the full length of the rat Nav1.7 protein sequence using DisorderEd Prediction CenTER (DEPICTER), which combines 10 popular algorithms for IDR predictions within the primary sequence, based on amino acid (aa) biophysical features for the protein's disordered ensemble (18). Results return a score between 0 and 1 for each residue, indicating the degree to which a given residue is part of an ordered or disordered region (residues with scores >0.5 are considered as disordered). Results revealed clear order-to-disorder transitions where Nav1.7 transmembrane (TM) domains and intracellular portions join, and scores indicate a disordered nature of Nav1.7 intracellular and terminal regions (FIG. 1A-1C). Specifically, the most extensive IDRs are in the intracellular loops (ICL), while protein TM domains are highly ordered.

Potential phosphorylation sites in the Nav1.7 sequence were identified using Disorder Enhanced Phosphorylation Predictor (DEPP) (19). Results showed that most potential phosphorylation residues (serine, threonine, and tyrosine with high DEPP scores) reside in Nav1.7-IDRs, particularly in the IDRs within the ICLI and ICL2 (FIG. 1D). Nav1.7-IDRs feature as potential protein-protein interaction (PPI) binding sites, suggesting these IDRs could contain key binding motifs or domains of the Nav1.7 regulatory signaling interactome (20). These observations predict that focusing on the Nav1.7-IDRs could be an avenue for identifying short peptides effective in modulating Nav1.7 channel function.

The potentially functional domains within the Nav1.7-IDRs (21) were further analyzed using SLIMPrints (http://bioware.ucd.ie/slimprints.html) (22), which predict short linear motifs (SLiMs) based on strongly conserved primary aa sequences, followed by filtering based on the prediction scores (22). The enumerated motifs predicted within Nav1.7-IDRs suggest many possible functional peptides as ‘hot-spots’ of functional IDDs, including proteolytic cleavage sites, ligand binding sites, post-translational modification (PTM) sites, and sub-cellular targeting sites. Nine peptides were designed computationally based on IDR scores and phosphorylation sites, and were the focus as 1.7iPA candidates for further testing (FIG. 1E, 1B).

Constructs of 1.7iPAs and Transfection Expression

AAV expression plasmids containing transgene expression cassettes encoding various GFP-1.7iPA chimeras were constructed. Specifically, the sequences for interchangeable iPA cloned with peptides were a linker sequence (GLRSRAQASNSAVDGTAGPGS) as we described previously (23), to form a chimeric transgene in a GFP-linker-iPA orientation transcribed by a hybrid human cytomegalovirus (CMV) enhancer/chicken β-actin (CBA) promoter. This generated pAAV-CBA-GFP-1.7iPAs (pAAV-1.7iPA) expression plasmids, in which the oligonucleotide encoding the interchangeable 1.7iPAs are inserted at the 3′ end of GFP (FIG. 1F). The predicted protein structure analysis of GFP1.7iPA1 by I-TASSER tool (https://zhanglab.ccmb.med.umich.edu/I-TASSER/) (24) shows an unfolded and extended, highly flexible structural ensemble of linker-1.7iPA1 (FIG. 1G), which is compatible with a well-exposed mode binding to targets. Similar structures were also identified by I-TASSER for other GFP1.7iPAs (FIG. 11).

Inhibition of Nav1.7 Current (I_{Na1.7}) in HEK1.7 Cells by 1.7iPAs

The stable expression of each construct was verified by transfection into HEK293 cells stably expressing human wild-type Nav1.7 (HEK1.7 cells), followed by immunoblots (IB). Representative tests for GFPlinker (GFP), 1.7iPAs (1, 2, 3, 4, 6) were shown (FIG. 1H, I). Initial screening experiments by whole-cell voltage-clamp of I_{Na1.7} in HEK1.7 cells, transfected with plasmids encoding nine 1.7iPAs (1.7iPA1-9), were performed to characterize the I_{Na1.7}. The presence of 9 different 1.7iPAs in HEK1.7 cells on peak I_{Na1.7} density (3 days after transfection) were summarized in FIG. 1F, in which the data points recorded by at least 2 replicates were combined. The results showed that 1.7iPA1, 4, and 6 produced ~68%, ~59%, and ~54% reduction of peak I_{Na1.7} density, respectively, while 1.7iPA2 increased peak I_{Na1.7} density (~35%). Transfection with plasmids expressing the GFPlinker and 1.7iPA3, 5, 7, 8, and 9 showed no significant effects on peak I_{Na1.7} density, compared to sham transfected (transfection with PEI but no plasmid) HEK1.7 cells. These experiments thus identified 1.7iPA1 and 1.7iPA4 (both derived from ICL1), as well as 1.7iPA6 (from ICL2), as effective iPAs (>50% I_{Na1.7} inhibition). We next focused on the validation of I_{Na1.7} inhibition and channel kinetics by 1.7iPA1, 4, and 6 on HEK1.7 cells in new experiments. These results replicated the prior screening testing results and showed that the steady-state activation and fast inactivation kinetics of Nav1.7 channels were not significantly affected in the presence of 1.7iPA1, 4, and 6 (FIG. 2). The 1.7iPA1 peptide is polyampholytic, enriched with 38.6% positively charged arginine or lysine (17/44), 23% of serine (10/44), 14% acidic residues (6/44); and is highly conserved between rodents and humans (FIG. 2F). Searching databases revealed that two serine phosphorylation and two lysine acetylation sites were assigned in high throughput (proteomic discovery mass spectrometry) studies (https://www.phosphosite.org) (25) and a nuclear localization signal was predicted by SeqNLS (http://mleg.cse.sc.edu/seqNLS/) (26). These analyses strongly suggest that 1.7iPA1 is a functional IDD peptide. Since 1.7iPA1 revealed higher inhibition of I_{Na1.7} and was highly homologous to other TTXs Nav subtypes (see further), we selected it as a prototype and named NaviPA1 for further ‘hit to lead’ characterization.

### Specificity of NaviPA1 Occupancy to Various Voltage-Gated Ion Channels

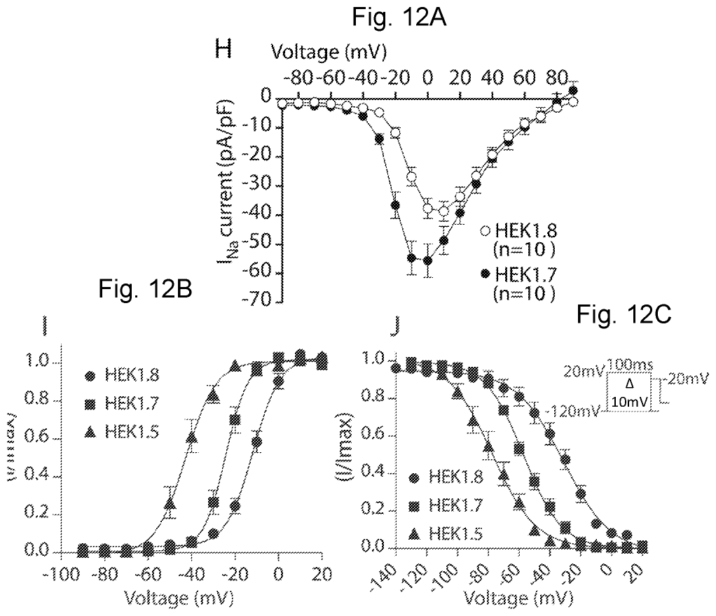

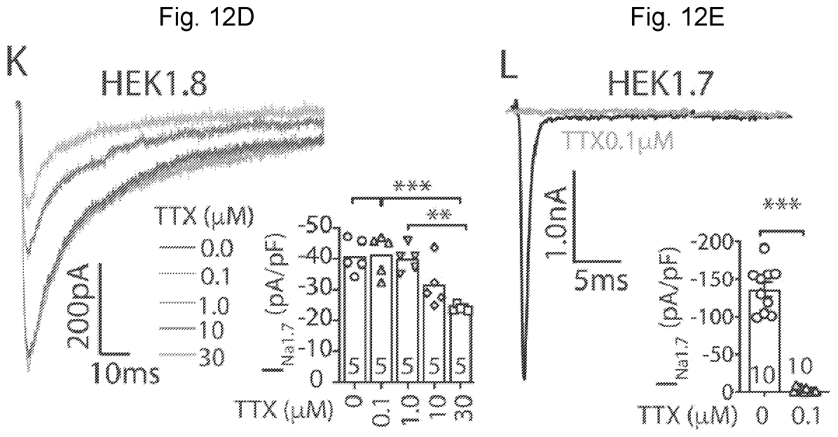

Development of Nav1.8 stable expression system based on HEK cells. To assess the potential of NaviPAL in affecting I_{Na} conducted by Nav1.8 channels, we developed stable expression of recombinant human Nav1.8 heterologous systems based on HEK cells (HEK1.8). Stable Nav1.8 expression was confirmed by immunoblots of Nav1.8a and Nab2 in the cells after at least 10 rounds of G418 selection (400-800 μg/mL), followed by single-cell isolation using BIOCHIPS Single-cell Isolation Chip (ThermFisher, Rockford, IL). Both Nav1.8a and Nab2 were found to be highly expressed in the cell membrane. Functional Nav1.8 expression was identified by the presence of slowly inactivating inward I_{Na} elicited by voltage steps from −140 mV to +80 mV during the whole-cell voltage-clamp recordings and the averaged peak I_{Na1.8} density in ~85% of the HEK1.8 was >0.5 nA, and I_{Na1.8} was sensitive to a Nav1.8 channel blocker, A803467 (Alomone, Jerusalem, Israel) and resistant to TTX (5 mM). We used this HEK1.8 cell line for the initial screening tests of the NaviPA1 on I_{Na1.8}. In comparison, I_{Na1.8} amplitudes in CHO-Nav1.8 cells were generally less than 100 pA, which was insufficient for our experimental needs (FIG. 12).

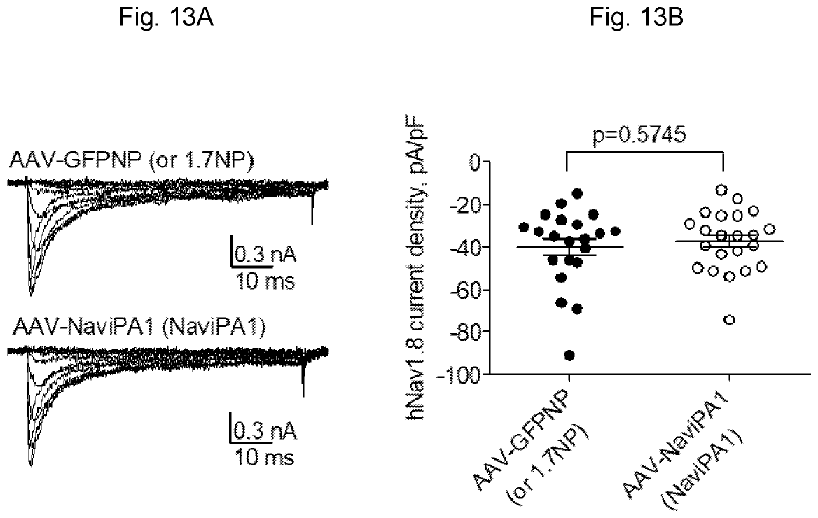

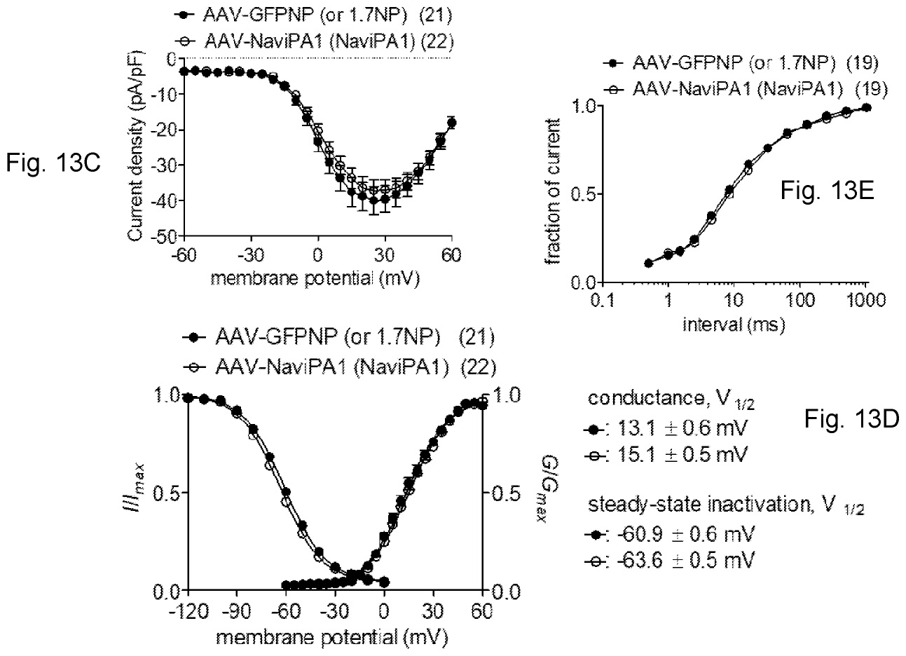

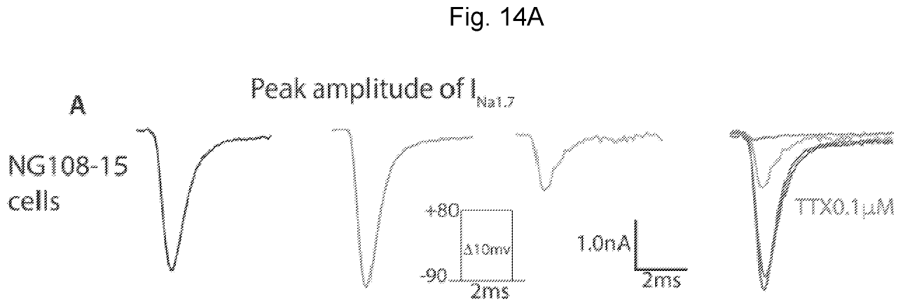

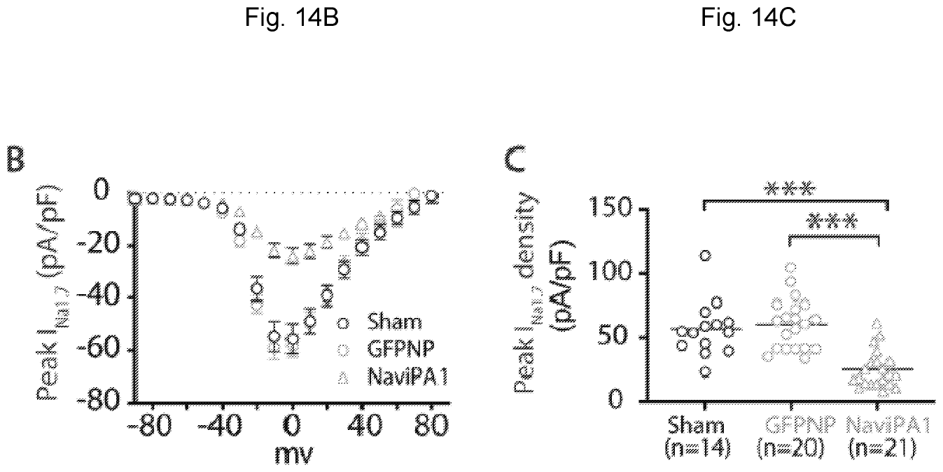

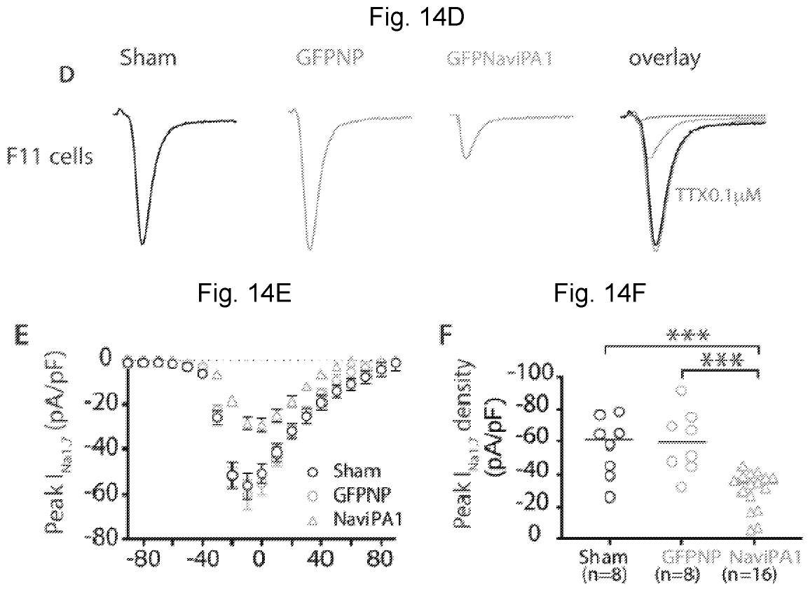

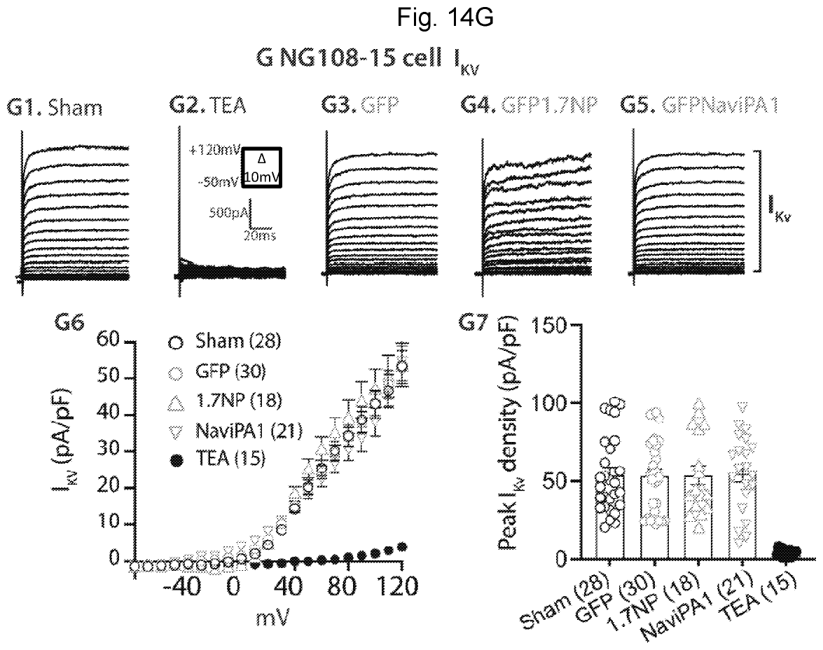

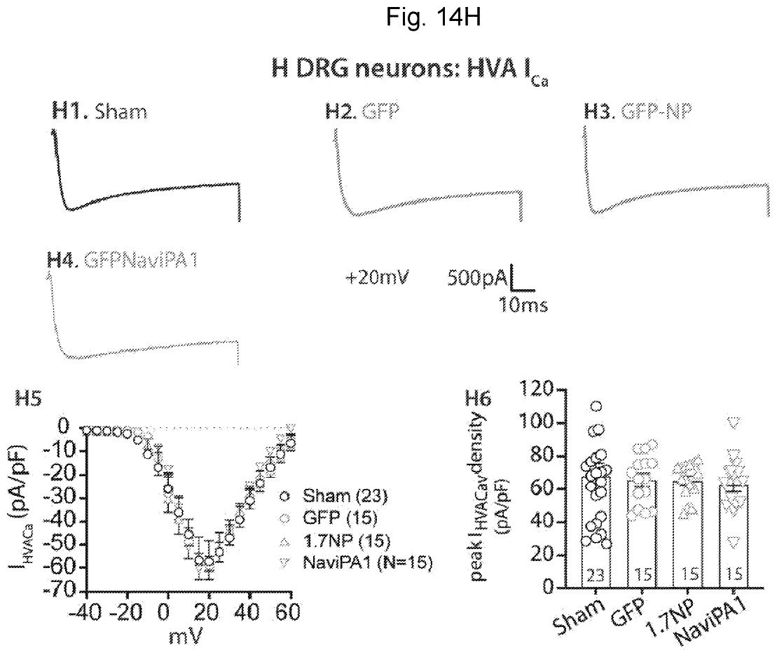

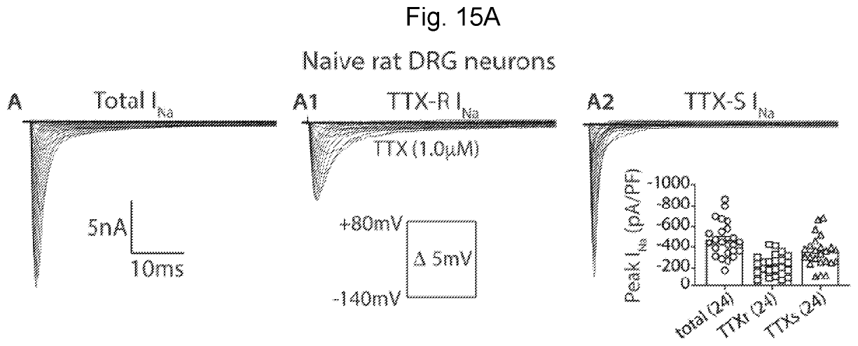

Selectivity of NaviPA1 on ion channel occupancy. Nav subtype stable cell lines based on HEK cells used for this experiment included HEK1.1, 1.3, 1.6, 1.5, and 1.8. Sequence alignments identified substantial homology of NaviPA1 with the corresponding sequences of TTXs Nav1.1, 1.3, and 1.6, but much less homologous to TTXr Nav1.5, 1.8, and 1.9 (FIG. 3A, B). Expression of NaviPA1 (fused to GFP) resulted in a significant block of I_{Na} conducted by fast-activating and inactivating Nav1.1, Nav1.3, and 1.6 (FIG. 3C-E). No effects on I_{Na1.5} and I_{Na1.8} were observed in the presence of NaviPA1 in the HEK1.5 and HEK1.8 cells (FIG. 3F-G) or in ND7/23 cells transiently transfected with Nav1.8 (FIG. 13). We did not test NaviPA1 against Nav1.1 and Nav1.9 channels as the expression cell line is unavailable; however, I_{Na1.9} inhibition by 1.7iPA1 is not expected because of sequence homology (not shown), while no effect on I_{Na1.9} is anticipated since there is no sequence homology of NaviPA1 to Nav1.9. The negative effects of NaviPAL on potassium current (BK I_{Kv}) were found in NG108-15 cells which naturally express potassium channels (12), and no effects on high-voltage activated (HVA) I_{Ca} were recorded on AAV-mediated NaviPA1 expression in DRG-PSNs. Potent INa1.7 inhibition by NaviPA1 was also confirmed in neuronal NG108-15 cells and F11 DRG-neuronal-like cells that naturally express Nav1.7. These experiments showed no effects of NaviPA1 on either BK potassium channels or HVA I_{Ca} (FIG. 14).

### AAV6-Mediated NaviPA1 Expression in DRG-PSNs Inhibits TTXs I_{Na} but not

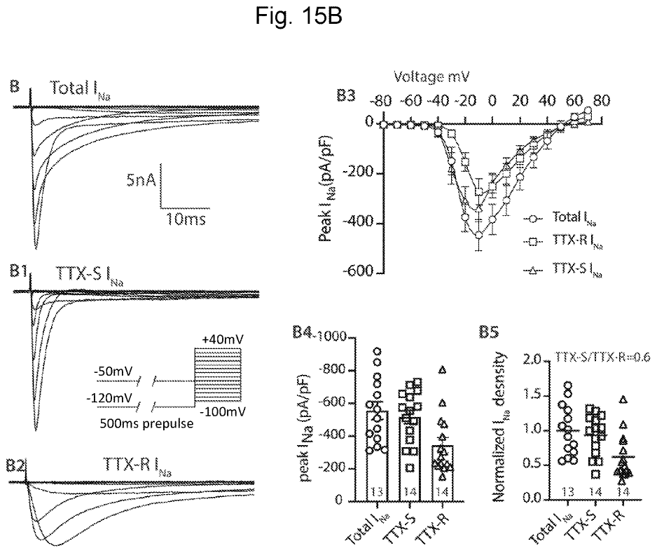

TTXr I_{Na}. Because no heterologous system or cell lines can fully mimic the in vivo conditions of sensory neurons, we further tested the functional inhibition of I_{Na} by NaviPAI in DRG-PSNs. AAV6 vectors encoding GFP-fused NaviPA1 were generated and injected into lumbar (L) 4/5 DRG of naïve rats (male), and acutely dissociated sensory neurons from DRG were tested at 4 weeks post-injection. AAV6 encoding GFPlinker and NP (1.7iPA3) which was derived from the N-terminus of Nav1.7 (FIG. 1) and showed no impact on I_{Na} after being transfected into HEK1.7 (FIG. 1, 2) were used as the control. A voltage protocol was adopted that demonstrates successful separation of TTXr I_{Na} (Nav1.8-like) and TTXs I_{Na} in dissociated DRG neurons (27, 28), comparable to the recordings after addition of TTX (1.0 mM) in bath solution (FIG. 15A, B). Whole-cell voltage-clamp recordings by the voltage protocol from small/medium-sized PSNs (≤35 mm) showed that AAV-mediated expression of NaviPA1 produced significant inhibition of total and TTXs I_{Na} whereas it produced no significant inhibition on TTXr I_{Na} (FIG. 4A-C).

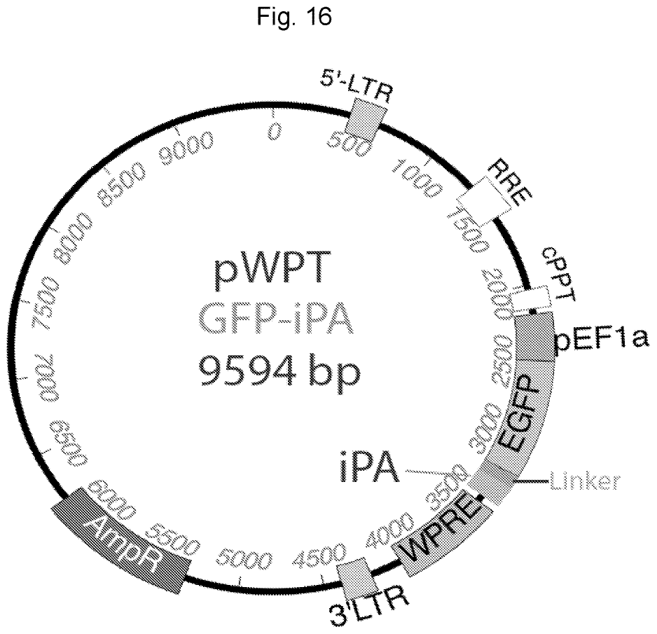

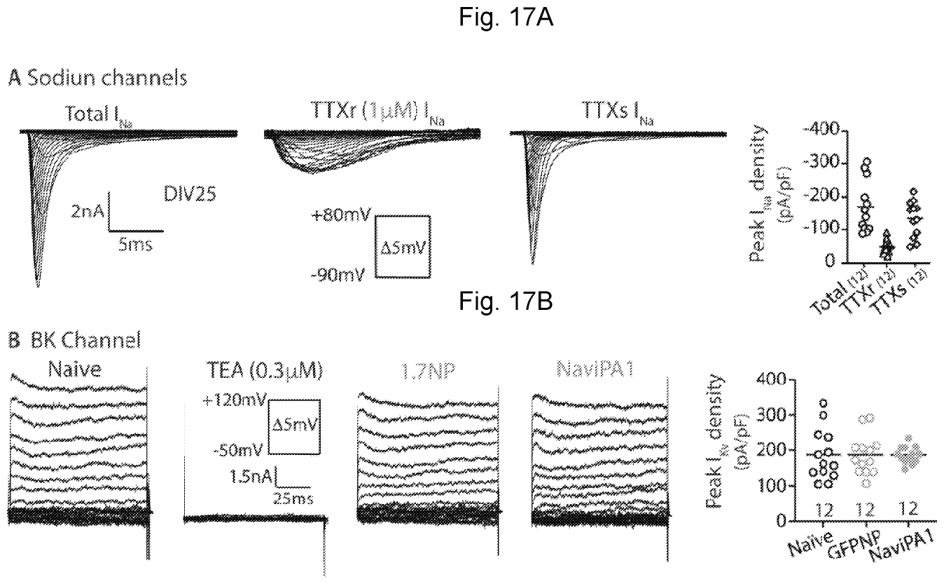

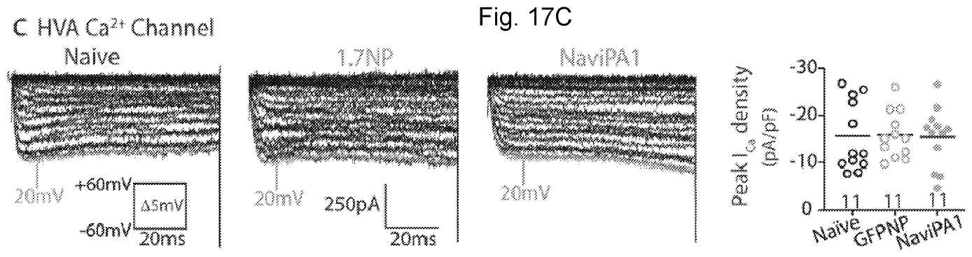

Inhibition of TTXs I_{Na} by NaviPA1 in human iPSC-derived sensory neurons. We used human induced pluripotent stem cells (iPSC)-derived sensory neurons (hiPSC-SNs, female, Anatomic, Minneapolis, MN) (29) to test whether inhibition of TTXs I_{Na} by NaviPA1 represents a meaningful and quantitative index of the functional lead in human sensory neurons. This also allowed examination NaviPA1 without potential overexpression effects in HEK-Nav cells. The hiPSC-SNs were differentiated to small-sized PSN morphology with a soma diameter around 20~25 mm and developed extensive neurites after 4-7 days in vitro (DIV) differentiation cultures, indicating that these cells were efficiently committed to the neuronal lineage. We used lentivector (LV-GFP) (FIG. 16) to test hiPSC-SN transduction efficiency. We have succeeded in expressing NaviPA1 and 1.7NP (control) in the differentiated hiPSC-SNs by LV transduction at multiple of infection (MOI)=5 (FIG. 4D, E). EP recordings were performed on the hiPSC-SNs (DIV25) with TTX (1 mM) in the bath solution and TTXr/TTXs I_{Na} were separated by a subtraction protocol (27). To prevent the TTX effect, a voltage manipulation similar to DRG neuron recording was used; additionally, a protocol to isolate somatic I_{Na}, a brief prepulse to voltage near spike inactivating hiPSC-SN axonal spike but not somatic spikes was adopted (30). Results showed that NaviPA1 significantly inhibited TTXs I_{Na} but not TTXr I_{Na} in differentiated hiPSC-SNs (DIV25) (FIG. 4F-G), comparable to rat DRG-PSNs. No effects were observed for BK I_{Kv} and HAV I_{Ca} recorded (DIV21) from hiPSC-SNs in the presence of NaviPA1 (FIG. 17). Results indicate that inhibitory efficacy of NaviPA1 on TTXs I_{Na} defined in cell lines and rat DRG-PSNs are translatable to human PSNs.

### Molecular Mechanisms of NaviPA1: An Initial Testing

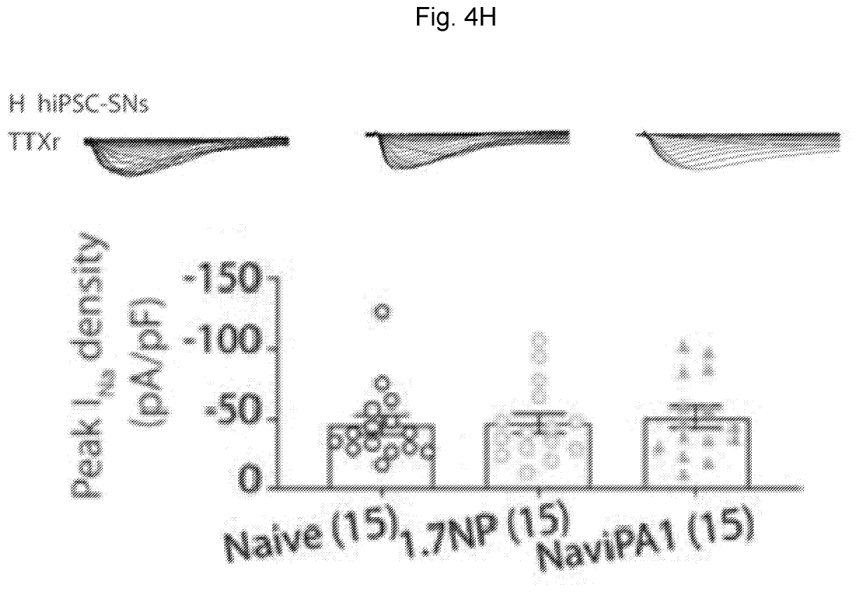

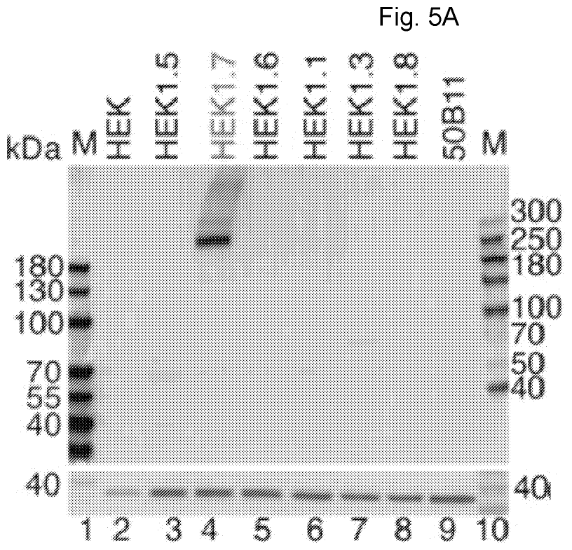

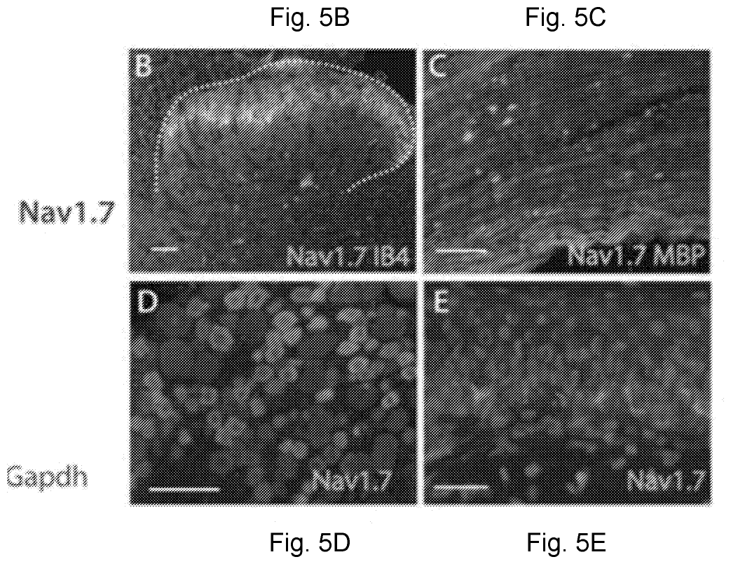

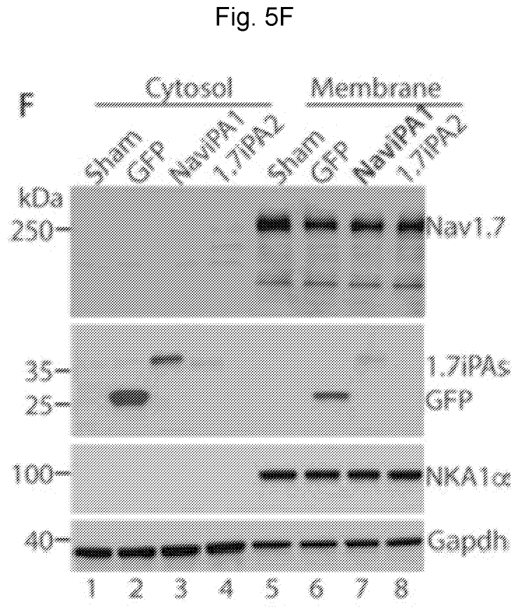

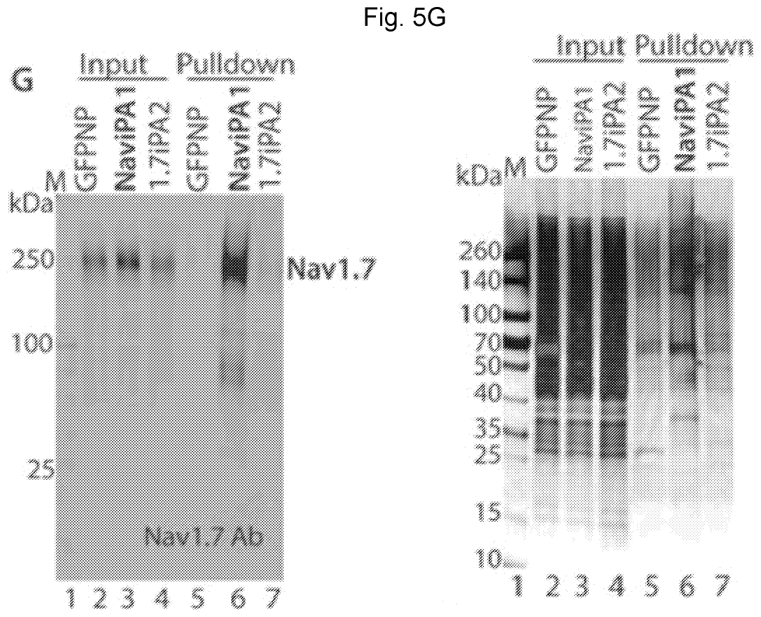

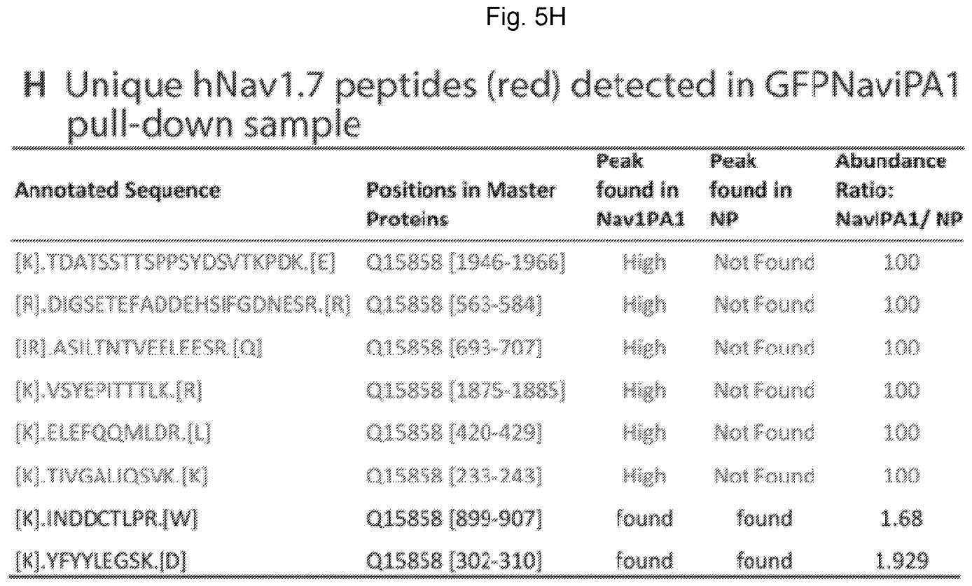

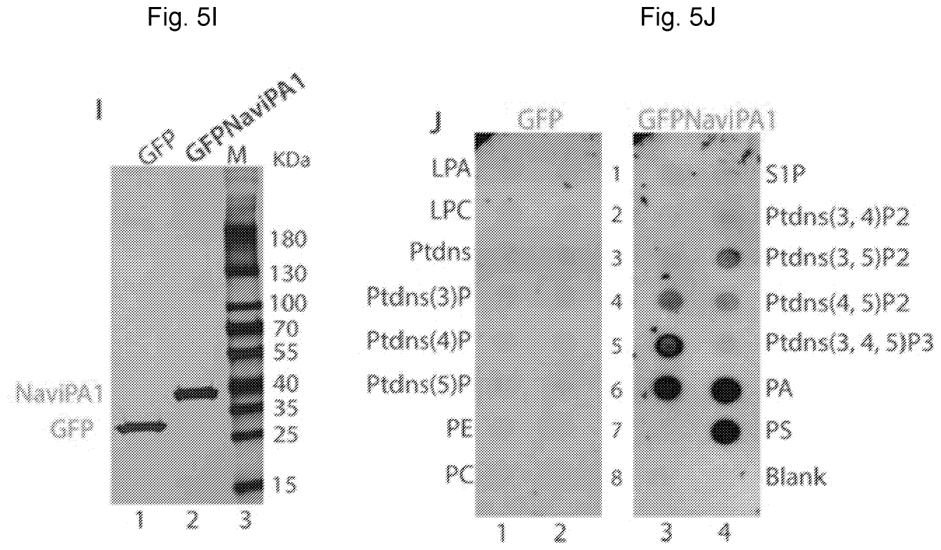

We first validated the specificity of Nav1.7 antibody by immunoblotting (IB) using the cell lysates prepared from naïve HEK cells, stable cell lines expressing different Nav isoforms, and 50B11 rat DRG neuronal cells. This Nav1.7 antibody (Alomone ASC-008) was raised by an antigenic peptide corresponding to amino acid residues 446-460 of rat Nav1.7 and no significant sequence homologous with other Nav isoforms. Results showed that the Nav1.7 antibody detected full-length Nav1.7 only in HEK1.7 cells, but not other Nav isoforms and 50B11 cells that naturally do not express physical and functional Nav1.7 (31) (FIG. 5A). By immunohistochemistry (IHC) on rat tissue sections, Nav1.7 expression was detected with high immunoreactive density in small/medium-sized PSNs using the Nav1.7 antibody, and Nav1.7 was also detected in spinal cord dorsal horn (SDH), sciatic nerve nodes of Ranvier, and cutaneous terminals in hindpaw (FIG. 5B-E), with the patterns similar to the prior report (32). These results confirmed the specificity of the Nav1.7 antibody to detect Nav1.7 expression by IHC and immunoblot.

Since Nav1.7 is an integral membrane protein, we therefore tested whether NaviPA1 expression in the HEK1.7 cells would interrupt Nav1.7 intracellular trafficking. Our results do not support this mechanism since no clear reduction of membrane Nav1.7 protein was evident in the fractionalized preparations, transfected with NaviPAL and controls (FIG. 5F). Studies have shown that IDRs in the membrane proteins engage in interactions with the membrane (33). To test whether NaviPA1 interference of Nav1.7 might be via direct block of Nav1.7, GFP affinity pull-down by ChromoTek GFP-Trap (ChromoTek, Rosemont, IL) was performed after transfection of GFP-NaviPA1 in HEK1.7 cells using GFPlinker (GFP) and GFP-1.7iPA2 (FIG. 1) as the controls. Cell lysates were prepared by a lysis buffer containing 0.5% Nonidet p40, a ‘non-denaturing’ mild lysis detergent, for preventing interaction breaking and maximizing the retention of NaviPA1-protein interactions (34). Immunoblots verified full-length Nav1.7 protein trapped in the GFPNaviPA1 pull-down sample but not in controls (FIG. 5G), and nLC-MS/MS detection of unique hNav1.7 peptides (FIG. 5H) confirmed hNav1.7 on the excised band from silver-stained sodium dodecyl sulfate-polyacrylamide gel electrophoresis (SDS-PAGE) gel of GFPNaviPA1 affinity pull-down sample. These results indicate that NaviPA1 block of Nav1.7 channel activation could be via binding to the Nav1.7 protein, i.e., an intra-molecular domain-domain interaction (intraDDI) (35). It has been reported that polybasic IDRs in transmembrane proteins preferably bind to negatively charged lipids (36, 37). We reasoned that NaviPA1 might be able to bind phosphoinositides, and this hypothesis was tested by using phosphatidylinositol phosphate (PIP) strips (Echelon PIP Strip, Salt Lake City, UT). GFPNaviPA1 and GFP (control) were transfected into neuronal NG108-15 cells, and cell lysates were prepared by a RIPA buffer containing 0.1% SDS and 1% Triton X100 (strong detergents) and 1% deoxycholate (anionic detergent) for maximally denaturing to break NaviPA1 PPI complex formations. Silver stain after SDS-PAGE gel showed clean purification of GFP and GFPNaviPA1 (FIG. 5J) and samples were applied to the PIP strips. Results (FIG. 5K) showed that GFP NaviPA1 was efficiently bound to a number of anionic PIPs, PIP2, phosphatidic acid (PA), and phosphatidylserine (PS). In contrast, affinity pull-down GFP did not show clear binding to lipid spots as previously reported (38). This is consistent with the reports that basic residues, often clustered in IDRs, can modulate membrane protein functions by binding via electrostatic interactions with lipids (39, 40).

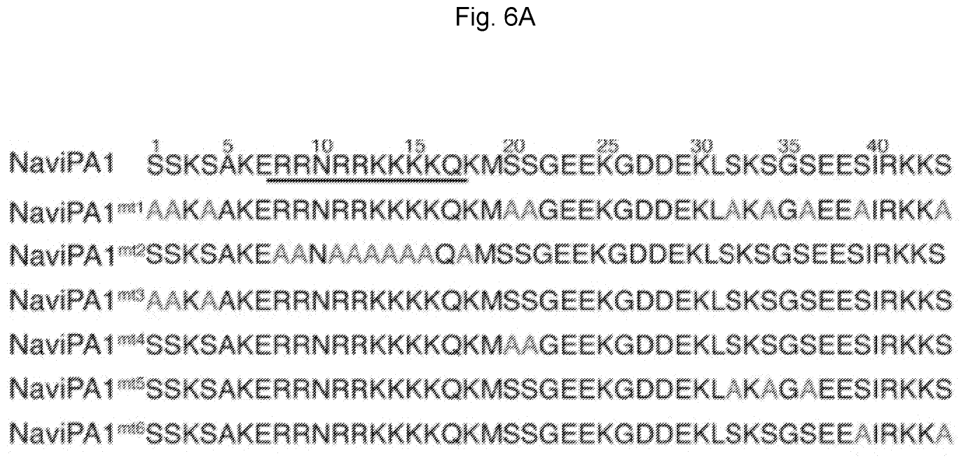

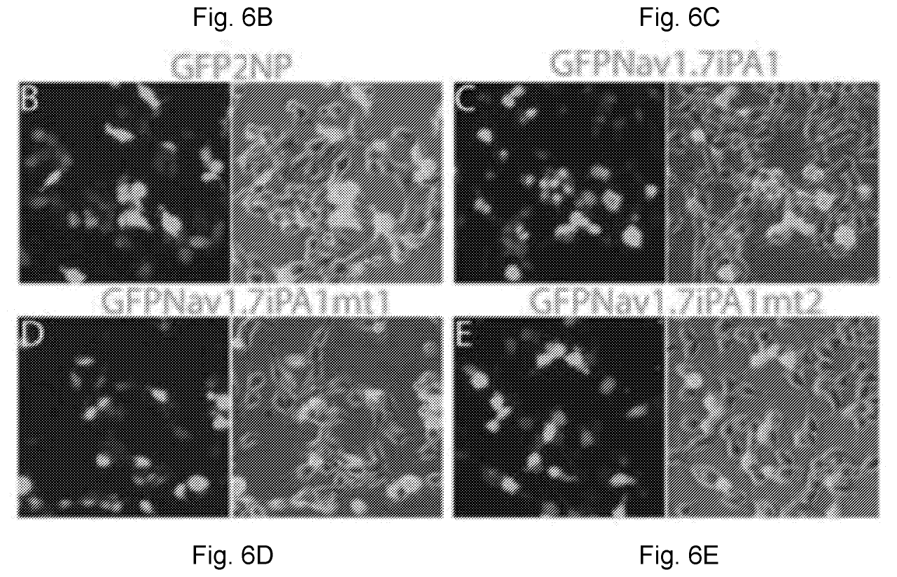

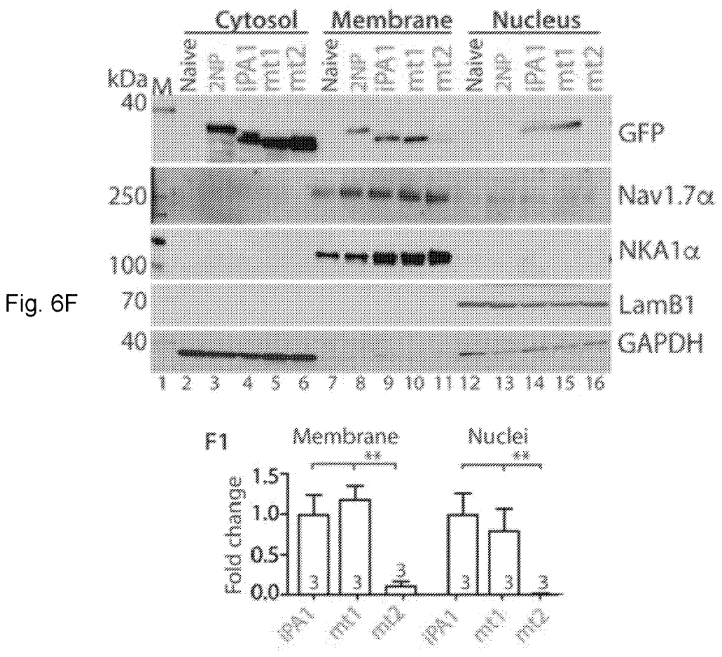

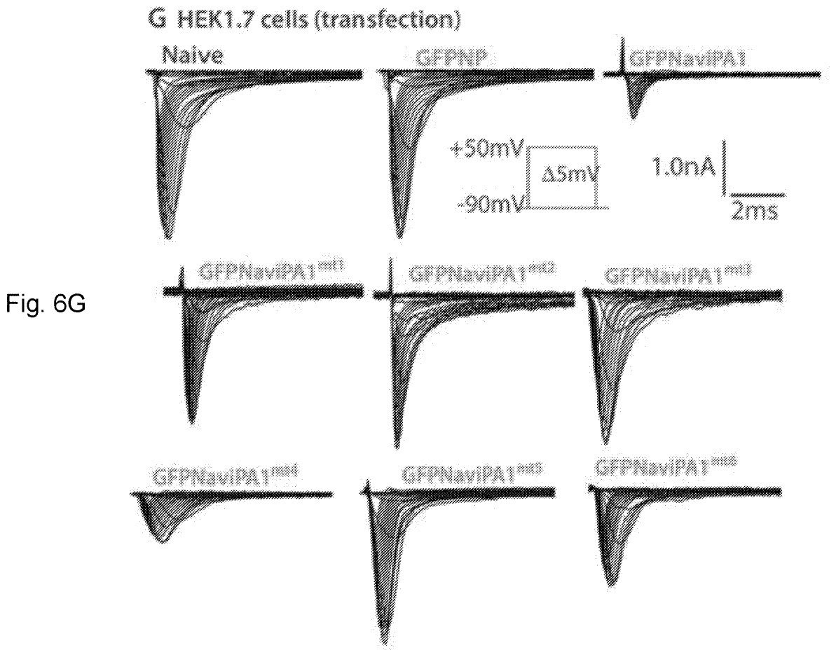

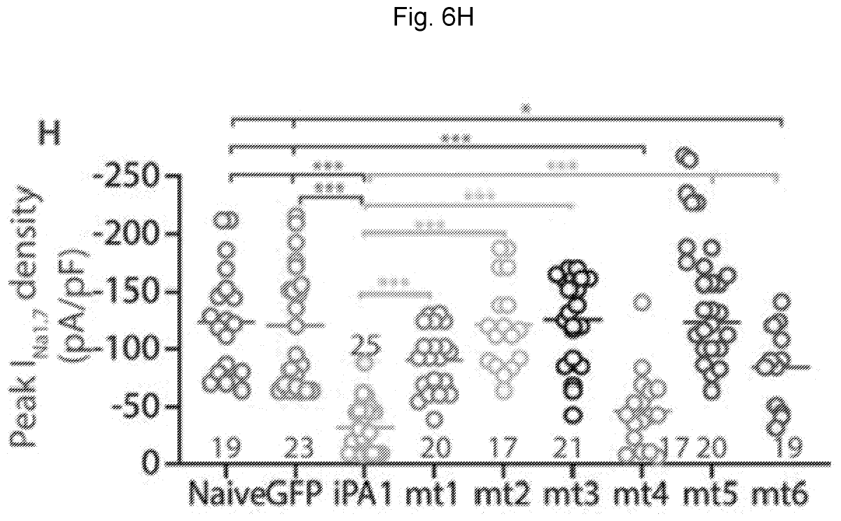

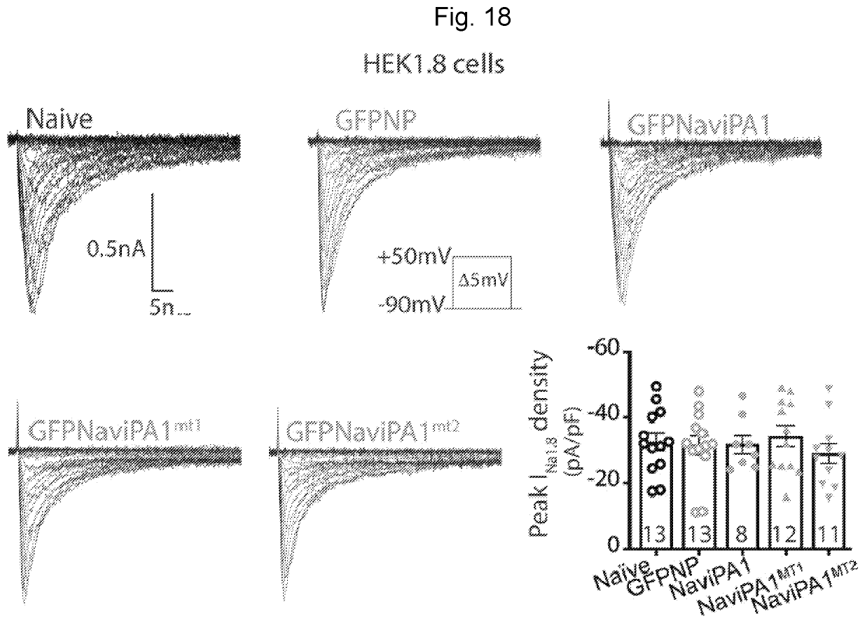

NaviPA1 is a polybasic arginine/lysine and serine-enriched peptide. It is reported that protein-conserved polybasic domains with adjacent serine PTMs often play roles in protein function (41-43). We designed experiments to examine the role of polybasic NLS and multiple adjacent polyserine in the function of NaviPA1. Initial tests were performed by generating NaviPAlmt1 (GFP-fused) in which alanine substitution for ten serine residues within NaviPA1 was made and NaviPAlmt2 (GFP-fused) was generated by alanine substitution for nine arginine or lysine within the predicted polybasic NLS of NaviPA1 (FIG. 6A). ICC showed that nuclear localization of NaviPA1 (HEK1.7 cell transfection) was observed in NaviPA1 and NaviPAlmt1 but diminished in NaviPA1mt2 (FIG. 6B-E). Immunoblots (FIG. 6F) revealed that NaviPA1 was detected in the extracted cytosol, membrane, and nuclear samples and that the membrane-binding and nuclear entry signals in NaviPAImtl were comparable to NaviPA1 but both vanished in NaviPAlmt2. Full-length Nav1.7 was enriched in the membrane samples as shown in FIG. 5F and the presence of NaviPA1, mt1, and mt2 in HEK1.7 cells appeared not to impede Nav1.7 protein membrane integration. Whole-cell voltage-clamp recording showed that I_{Na1.7} in the presence of NaviP Almt1 and mt2 was comparable to naïve and GFPNP transfected HEK1.7 cells, suggesting that both polybasic arginine/lysine and multiple adjacent serine residues were required for NaviPA1 inhibitory effect on Nav1.7 current. To further map the critical serine sites, we generated additional NaviPA1mt3-mt6 with alanine substitution for dual or triple serine residues (FIG. 6A). Whole-cell voltage-clamp recording showed that NaviPAlmt3 and 5 with alanine substitution at different serine sites lost inhibitory effects on I_{Na1.7} after transfection to HEK1.7 cells while mt4 and mt6 showed a significant block of I_{Na1.7} (FIG. 6G, H). As expected, NaviPAImtl and 2 did not change I_{Na1.8} after transfected to HEK1.8 cells, similar to NaviPA1 (FIG. 18). These data suggest that conserved polybasic NLS and multiple adjacent serine residues within the NaviPA1 are synergistic for INa1.7 inhibition. The polybasic motif determines for polar association with the plasma membrane and nuclear entry of disordered NaviPA1 peptide and multiple adjacent serine residues are required for NaviPA1 inhibitory effect to I_{Na1.7}. However, the full-length Nav1.7 membrane integration, which is determined by its TM domains but not intracellular sequences, was unaffected in the presence of NaviPA1 (FIG. 6F).

Future delineation of the properties of serine and other residue PTMs within NaviPA1 the molecular mechanisms underlying inhibition of various TTXs I_{Na} and whether the presence of NaViPA1 might undermine TTXs Nav channel activity via decoying, diminishing PTMs in the full-length protein and/or altering intradomain effects these events are of interest from both pathophysiological and therapeutic perspectives. Our goal in this study is to develop a strategy of peripherally-targeted analgesia via AAV-mediated sensory neuron-specific inhibition of sodium channels. Therefore, in the following in vivo experiments, we focused on testing whether DRG-PSN-targeted expression of NaviPA1 is effective in attenuating neuropathic pain behaviors.

Analgesia after Intraganglionic Delivery of AAV-NaviPA1 in Rats after TNI

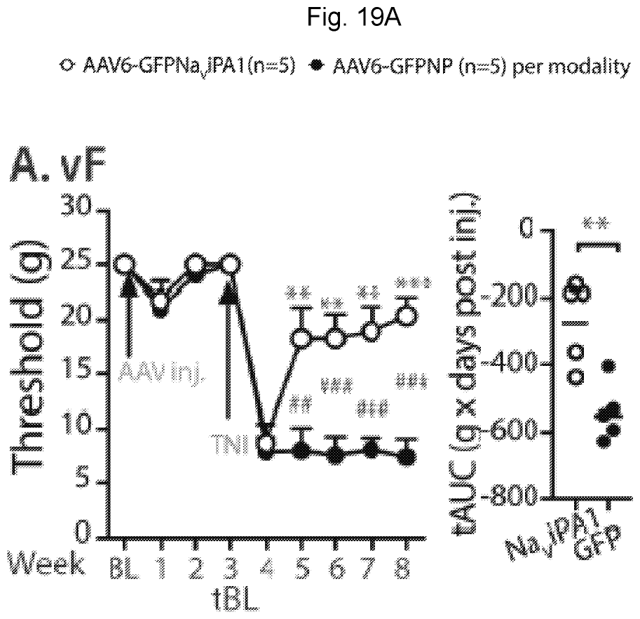

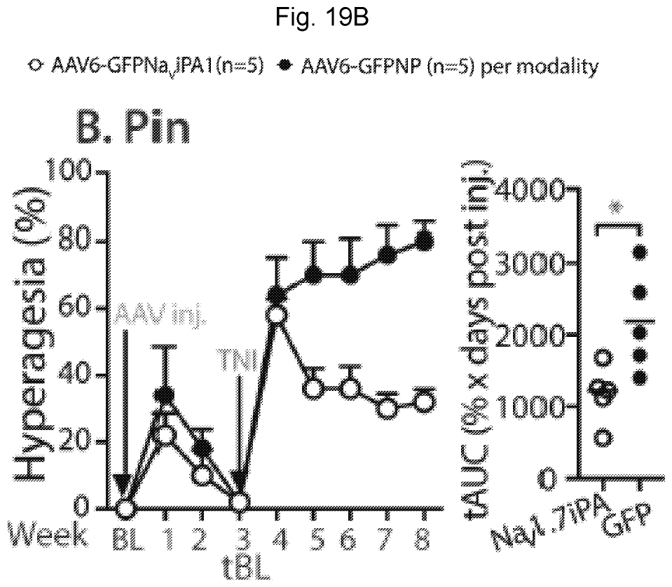

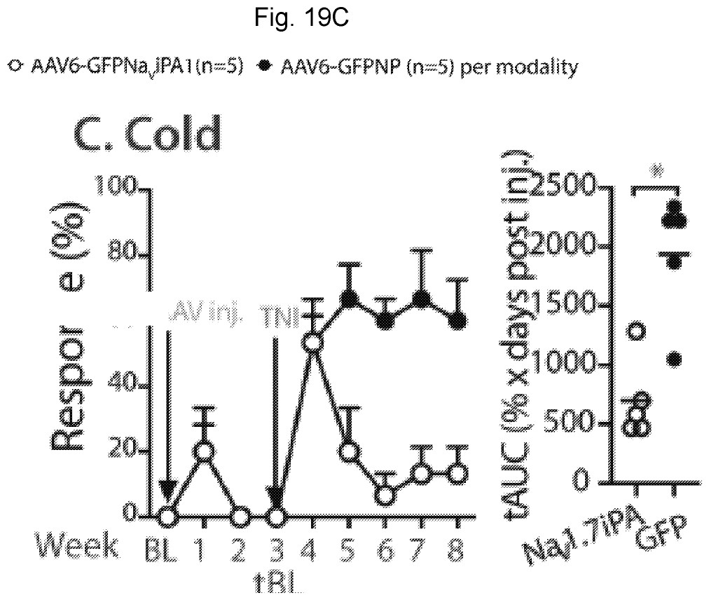

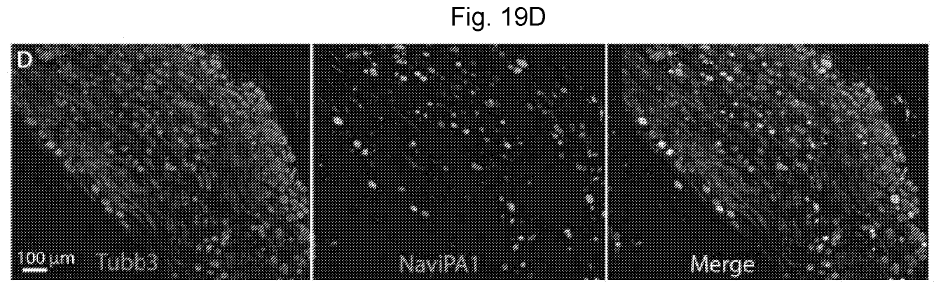

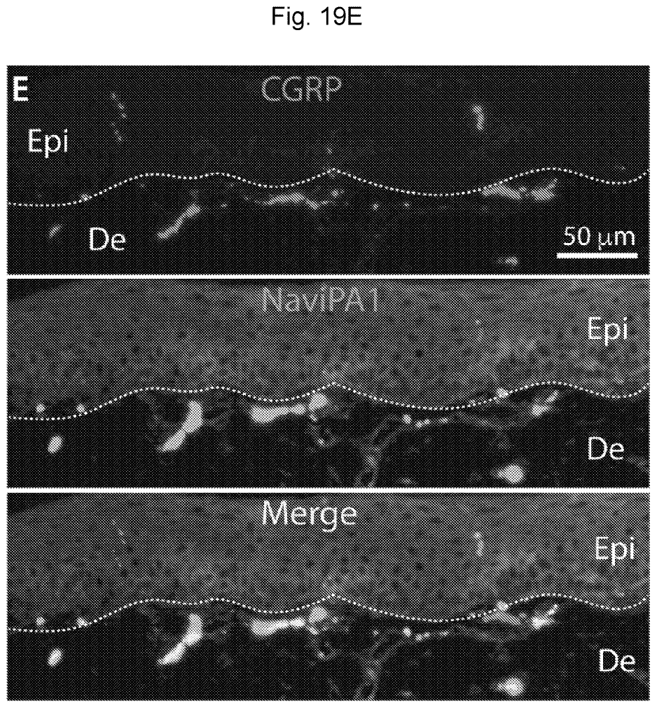

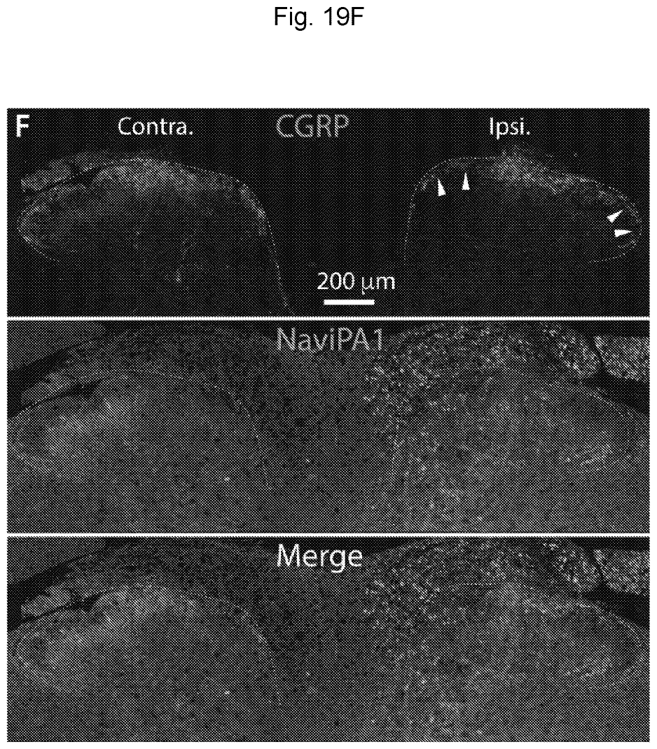

We first conducted a pilot in vivo analgesia testing. High-titer and high-purity of AAV6-GFPNaviPA1 (AAV6-NaviPA1) and control AAV6-GFPNP (AAV6-NP) were generated and injected into the L4/5 DRG of adult male rats. Three weeks after DRG-AAV injection, TNI was induced, and subsequent sensory behavior evaluation was performed weekly for an additional 5 weeks, after which tissues were harvested for IHC characterization of transgene expression. Results (FIG. 19) showed that AAV6-NaviPAI injection reduced TNI-induced mechanical and cold sensitization. IHC revealed efficient NaviPA1 (fused to GFP) expression in DRG neurons and their peripheral (cutaneous) and central terminals (SDH). These data indicate that sustained expression of the NaviPA1 selectively in the PSNs of the pathological DRG after TNI prevented development of pain behaviors.

### Treatment of Established Neuropathic Pain by DRG-AAV6-NaviPA1 in Male Rats

We next extended experiments to evaluate the effectiveness of DRG-AAV6-NaviPA1 in a more clinically relevant design for reversal of established pain behaviors, including both evoked responses as well as spontaneous ongoing pain following TNI. In the experimental design, the sensitivity to mechanical and thermal cutaneous stimulation was assessed at baseline and weekly after TNI for 2 weeks before AAV injection. Thereafter, rats were randomized to receive DRG injection of either AAV6-NaviPA1 or control AAV6-NP into the L4/L5 DRG ipsilateral to TNI, after which sensory behaviors were evaluated weekly for additional 6 weeks. As a terminal experiment, Gabapentin (GBP,100 mg/kg, i.p.)-induced conditioned place preference (CPP) test was performed in both groups to evaluate spontaneous pain (12, 44). Behavior measures before AAV injection on the 14^{th} day after TNI were used as a treatment baseline (tBL) to evaluate effectiveness of vector treatments (FIG. 7A, B). Tissues were harvested for IHC characterization of transgene and target gene expression and for whole-cell current-clamp of neuronal excitability on dissociated DRG neurons.

All rats developed multiple modalities of pain behaviors 2 weeks after TNI, including lowered threshold for withdrawal from mild mechanical stimuli (vF), more frequent hyperalgesic-type responses after noxious mechanical stimulation (Pin), and hypersensitivity to heat and acetone stimulation. These behaviors persisted after injection of the control AAV6-NP during the 6 weeks of observation course. In contrast, rats injected with AAV6-NaviPA1 showed a gradual reversal of these changes, which were maintained throughout and predicted to outlast the observation period (FIG. 7C-F). For our protocol of treating existing pain, we used the measures on the 14^{th} day after TNI and before AAV treatment (tBL) as the peak pain intensity (100%), and the measures of each sensory modality after treatment were normalized to the measures at the tBL and the percentage of pain relief for each modality at multiple time points was calculated (FIG. 7C1-F1). Summed average pain relief in the 6-week treatment course showed 52%, 49%, 69%, and 67% reduction of vF-, Pin-, Cold-, and Heat-stimulated mechanical and thermal pain behaviors, respectively (FIG. 7G). Using a biased CPP paradigm (45), the effect of AAV-NaviPA1 treatment on spontaneous pain was evaluated. None of the animals in either group was excluded from study because of their baseline preference/avoidance for a chamber (45). A significant GBP-induced CPP effect was observed in the TNI rats injected with AAV6-NP, while there was no significant difference in the time spent in the initially nonpreferred chamber during baseline vs. testing period in AAV-NaviPA1 treated TNI animals, indicating that AAV-NaviPA1 treatment significantly relieved on-going spontaneous pain (FIG. 7H).

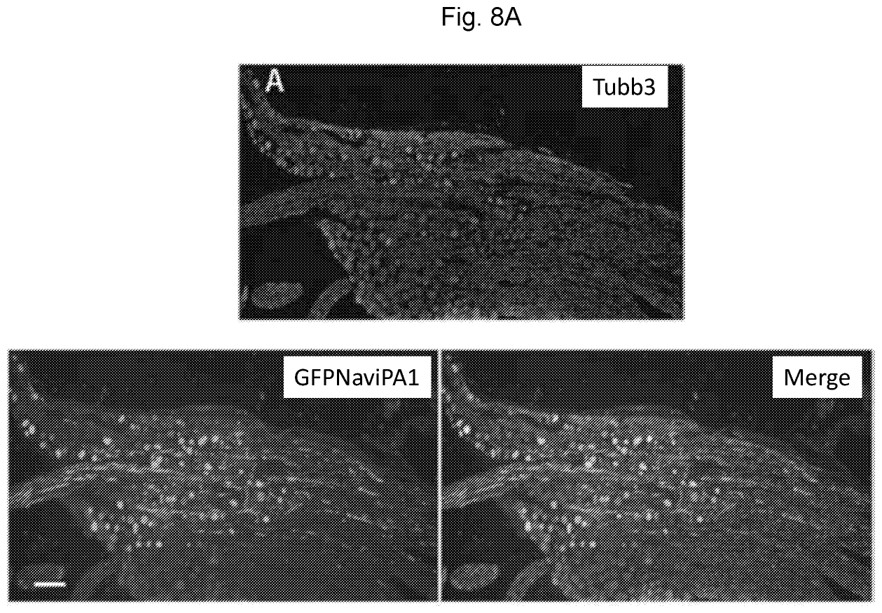

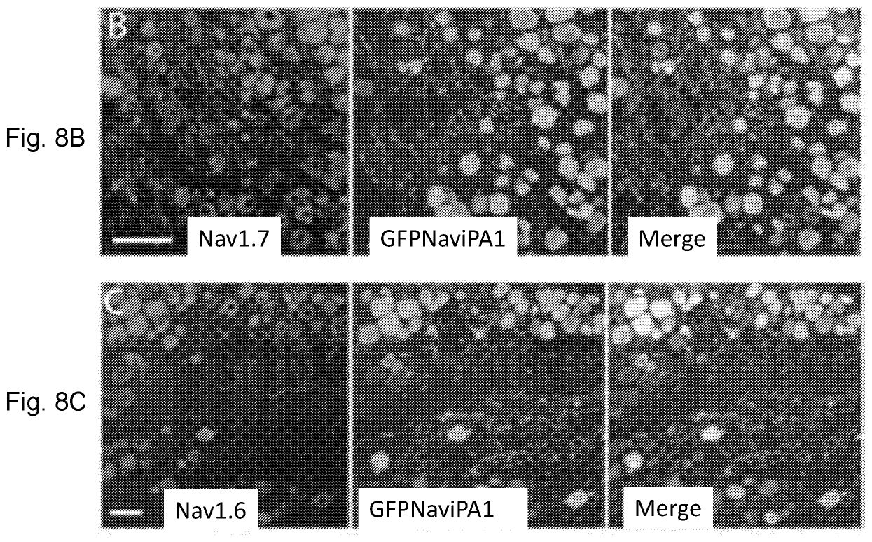

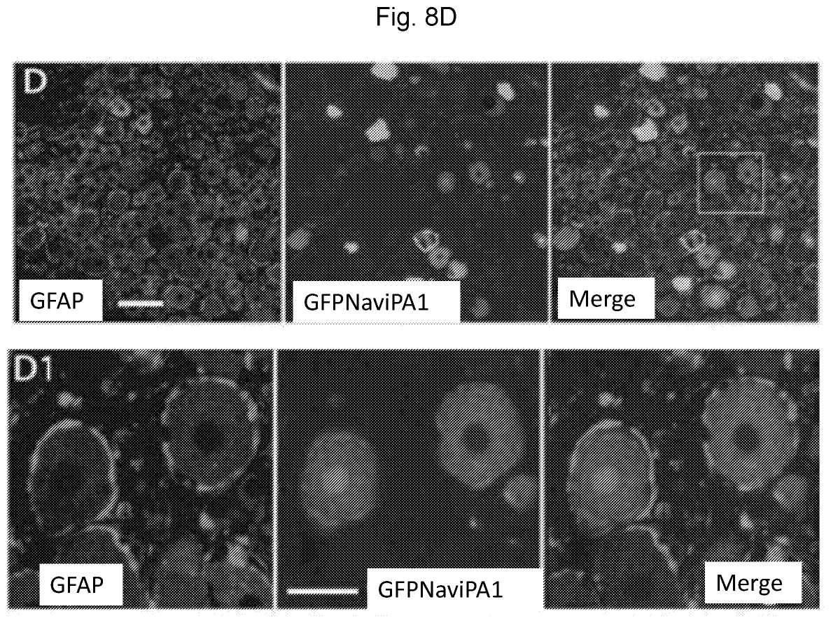

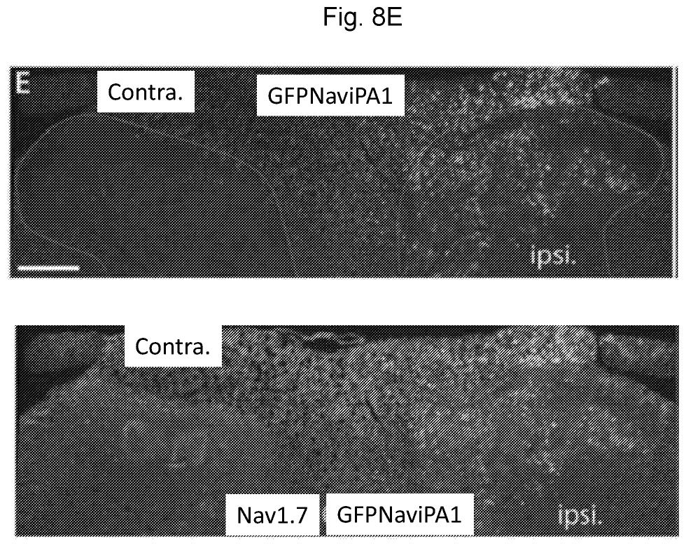

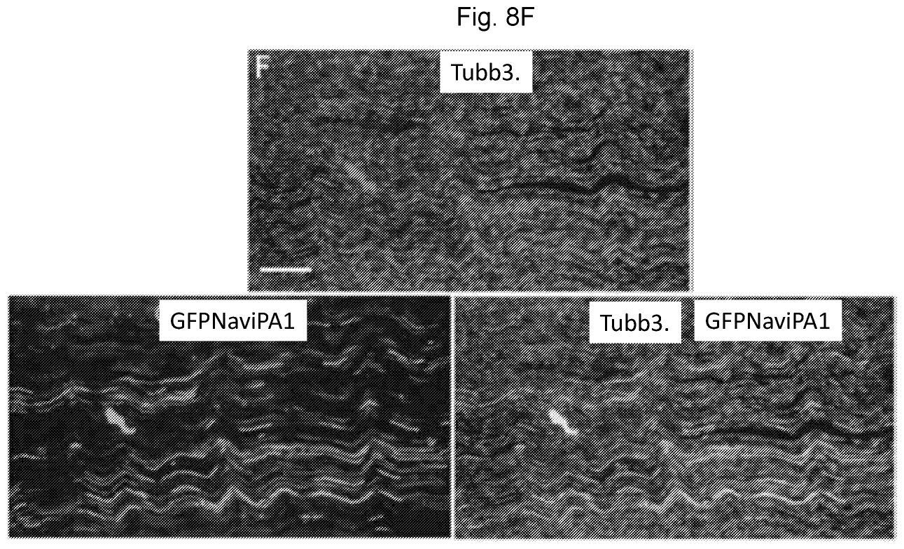

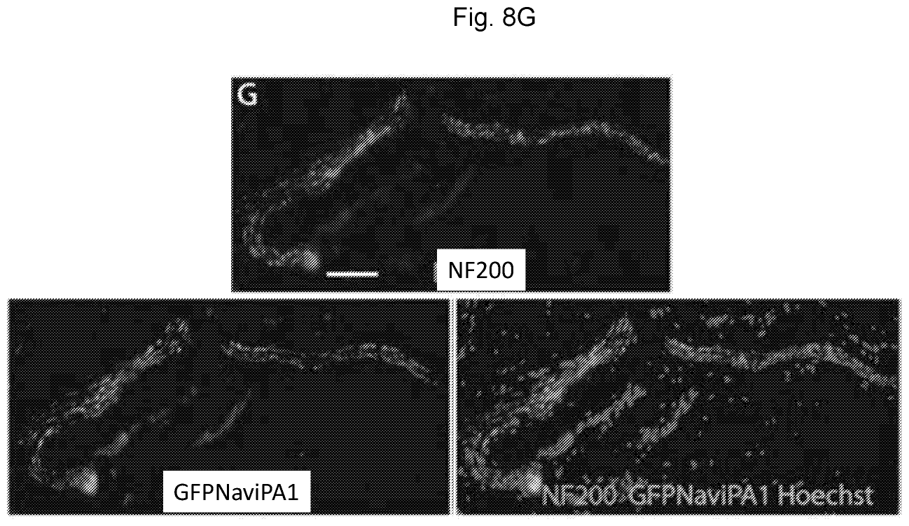

Histological examination (FIG. 8) determined the in vivo transduction rate for AAV6-NaViPA1 in the 6^{th} week after vector injection. The NaviPA1-positive neurons (GFP) comprised 37±13% (1283 out of 3447 total neuronal profiles) identified by a pan-neuronal marker β3-tubulin (n=6 DRG, 3-4 sections per DRG, selected as every fifth section from the consecutive serial sections). Transduced DRG neurons included the full-size range of the PSNs that also expressed Nav1.7 and Nav1.6, and expression showed multiple subcellular localizations, preferably in PSN cytosol. Positive GFP signals were not detected in GFAP-positive perineuronal glial cells. GFP signals were also detected in the ipsilateral dorsal horn, sciatic nerve, and cutaneous afferent terminals.

These findings together demonstrate that DRG injection of AAV6-encoded NaviPA1 induced NaviPA1 expression restricted to the PSNs of injected DRG and their peripheral and central processes. This strategy via AAV6-mediated expression of NaviPA1 selective in the sensory neurons of the anatomically segmental DRG responsible for pain pathophysiology has clear analgesic effectiveness in normalizing the established peripheral hypersensitivity for both evoked and spontaneous pain behavior in the rat model of peripheral injury-induced neuropathy.

### Reversal of PSN Hyperexcitability by AAV6-NaviPA1 Treatment (Male Rats)

Increased excitability of nociceptive PSNs is a fundamental process underlying neuropathic pain (46). We therefore examined whether AAV6-NaviPA1 treatment reverses the enhanced neuronal excitability of nociceptive PSNs following TNI (12, 47), using the whole-cell current-clamp AP recording of DRG dissociated neurons from rats after the treatment protocol shown in FIG. 7B. Although TNI results in DRG containing co-mingled injured and uninjured axons, nerve-injury can induce an increase of voltage-gated ion channel activity in both axotomized neurons and adjacent intact neurons, leading to similar electrophysiological (EP) changes and increased discharge frequency in axotomized and neighboring intact DRG neurons (48, 49), possibly through interneuronal signaling and coupling (50). We therefore recorded from randomly chosen small/medium-sized neurons (<35 μm in diameter) (51) in the cultures from dissociated L4 and L5 DRG. Transduced neurons were identified by GFP fluorescence, and excitability was evaluated by measuring rheobase and repetitive action potential (AP) firing during 250 ms current pulses stepping from 100 pA and 280 pA current injection. Results showed that the averaged rheobase in the neurons from TNI rats was significantly decreased and, in response to a step stimulus, the frequency of APs evoked in neurons from TNI rats was significantly increased, compared to sham controls. These were normalized in the transduced neurons after AAV6-NaviPA1 treatment, whereas NP-transduced neurons had no significant effects (FIG. 9). These findings indicate that reversal of nerve injury-induced sensory neuronal hyperexcitability by NaviPA1 may contribute to its analgesic effects in attenuation of neuropathic pain behaviors, i.e., conduction block of TTXs Nav ion channels selectively in PSNs leads to a substantial decrease in neural excitability, resulting in mitigation of pain behaviors.

### Analgesia of DRG-AAV6-NaviPA1 Treatment in Female TNI Rats

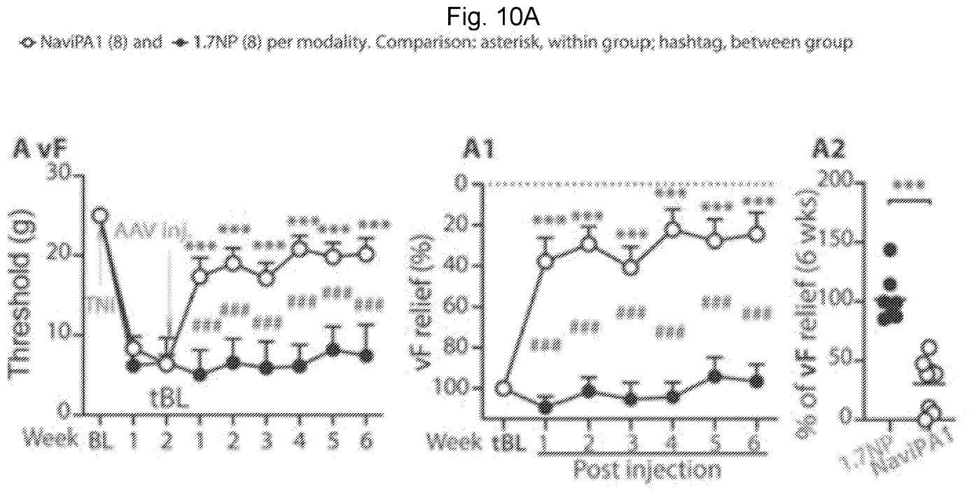

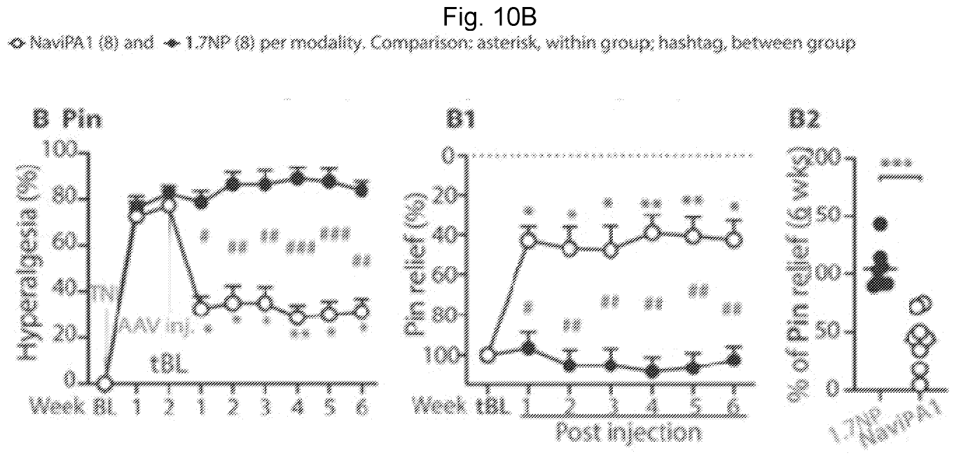

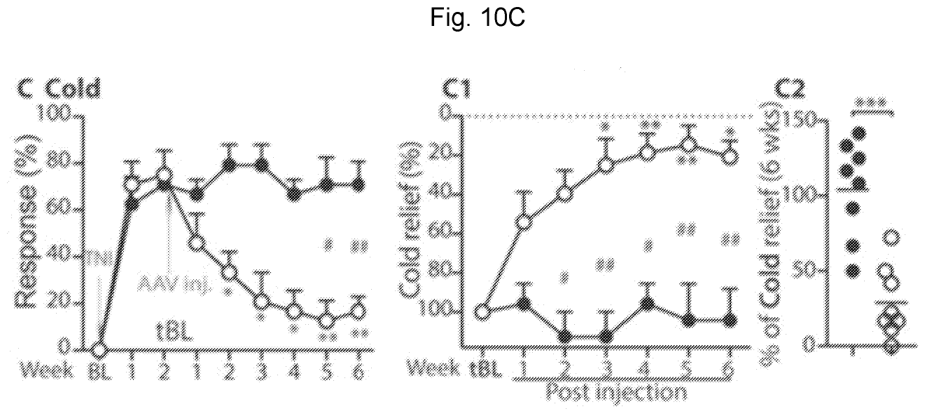

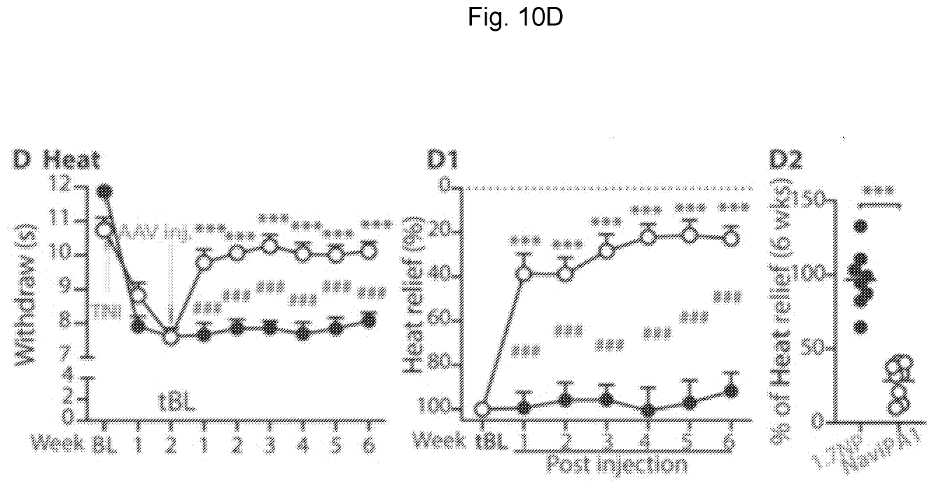

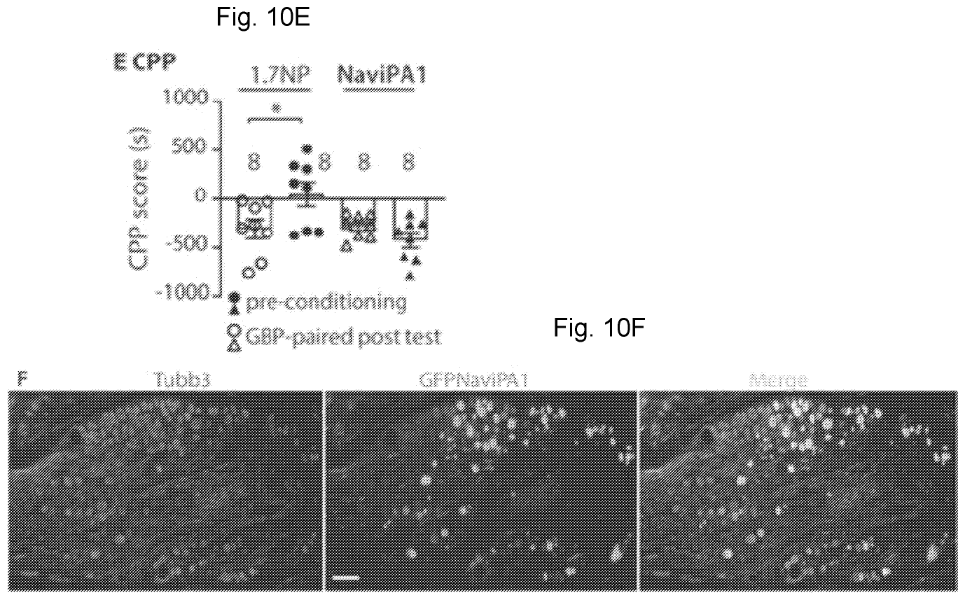

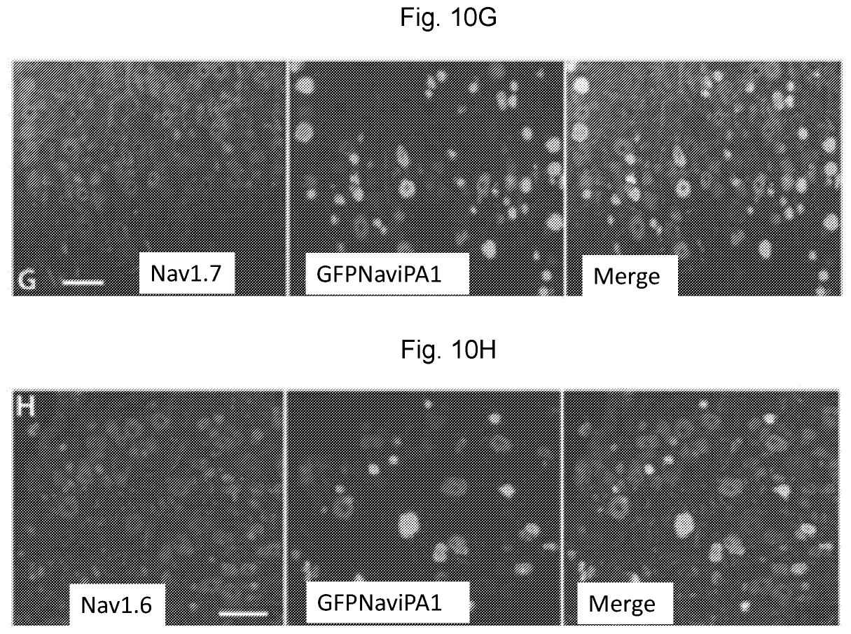

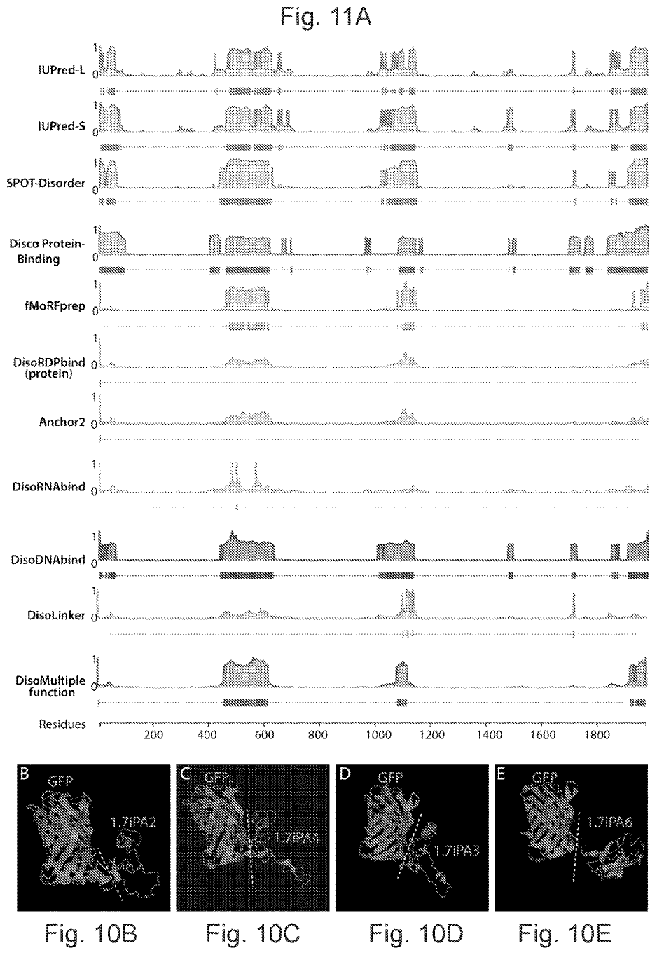

Sex differences exist in experimental and clinical pain and in responsivity to interventions (52). We therefore next tested whether DRG-AAV6-NaviPA1 treatment is also effective in attenuating hypersensitivity induced by TNI in female animals, using the protocol similar to the tests in male animals (FIG. 7). The same batch preparation of AAV6-NaviPA1 and AAV6-NP tested in male rats was used for injection. Results showed that the female rats displayed similar phenotypic development of hypersensitivity after induction of TNI to male rats and that both evoked mechanical/thermal hypersensitivity and GBP-CPP responses were normalized after AAV6-NaviPA1 treatment, demonstrating comparable analgesic effects (FIG. 10A-E) to the male animals. IHC on the DRG sections from female TNI rats 6 weeks after AAV6-NaViPA1 injection also revealed GFP-NaviPA1 expression profile comparable to male rats (FIGS. 9F-G), and the in vivo transduction rate was 39±25% (766 out of 1983 total Tubb3-positive neuronal profiles), however, transduction rate in DRG neurons was not quantified. Thus, although not rigorously compared, treatment effects were comparably concordant between the sexes, suggesting that a sexual dimorphism seems not apparent for both pain behavior phenotypes after TNI and in responsivity to DRG-AAV6-NaviPA1 treatment in our studies (12).

### DISCUSSION

Sustained peripherally targeted analgesia without risk of addiction is a global unmet medical need (53). Nav1.7 is currently a leading target for analgesic pharmaceutics. However, ample evidence demonstrates that multiple sensory neuronal Navs contribute to nociceptive electrogenesis and pain pathogenesis (15, 54). Here, we reported that targeting Nav-IDRs facilitated discovery of NaviPAs. A prototypic NaviPA1, initially derived from Nav1.7, is highly conserved in sequences among TTXs Navs, and accordingly, demonstrated multipronged inhibitory feature to TTXs I_{Na} conducted by Nav1.7, Nav1.6, Nav1.3, and Nav1.1 but no effect on TTXr I_{Na} conducted by Nav1.8 and Nav1.5. NaviPAI expression in DRG-PSNs produced selective inhibition of TTXs I_{Na} but not TTXr I_{Na}. DRG delivery of AAV6-encoded NaviPA1 significantly attenuated established nerve injury-induced pain behaviors in male and female animals for both evoked mechanical and thermal hypersensitivity and ongoing or spontaneous pain behaviors, the symptoms commonly found in patients suffering from multiple types of painful neuropathy (55). Additionally, blockade effects of TTXs I_{Na} by NaviP A1 were replicated in the hiPSC-SNs, supporting a translational potential. Because several different types of Navs in sensory neurons combine to trigger nociceptor electrogenesis required for AP trains (1), block of several of these specific in DRG-PSNs is conceived to be a therapeutical advantage for neuropathic pain.

Chronic pain in almost all cases is maintained by ongoing afferent hyperactivity originating from peripheral pathological sources (56-58). Thus, development of novel peripheral-acting strategies for pronociceptive Nav inhibition in the PSNs would be an ideal approach for clinical pain treatment (2, 59). Our strategy described here includes a novel approach by which highly selective and nontoxic NaviPA1 is designed and developed from Nav-IDRs, which is delivered by using AAV to the pathological DRG. PSN-restricted inhibition of multiple pronociceptive TTXs Navs is predicted to have advantages for DRG-targeted analgesia, as a recent expert commentary states that “disappointing analgesic pharmaceutics after a single Nav1.7 inhibition might correlate to the facts that the excitability of neurons is determined by several different Nav channels and targeting just one may not be sufficient by itself” (60). It is known that human subjects and animal models that are heterozygous for null mutations of Nav1.7 are normal in sensory phenotypes. Thus, AAV-mediated NaviPA1 expression restricted in DRG-PSNs may induce analgesia via a combined partial inhibition of Nav1.7, Nav1.6, Nav1.3, and Nav1.1 (likely including Nav1.1), while avoiding undesirable side-effects otherwise due to global distribution of small molecule inhibitors. Although PSN somata in DRG are anatomically isolated from each other and are not synaptically interconnected, most DRG-PSNs are transiently depolarized when axons of neighboring neurons of the same ganglion are stimulated repetitively (61). This coupled activation occurs among various-sized neurons including small-diameter nociceptors and large-diameter low-threshold mechanoreceptors (50). Therefore, although AAV produces incomplete sensory neuron transduction, transduced neurons can induce a reduction of pronociceptive ion channel activity in both transduced neurons and adjacent non-transduced neurons, leading to similar electrophysiological changes. Another possible advantage is that, unlike gene therapy strategies such as RNAi (62) and CRISPIR-dCas9 or ZEN epigenetic suppression (9) that irreversibly reduce the production of a target protein, which is potentially problematic (63); AAV-mediated NaviPA1 expression selective in PSNs provides sustained and restricted blockade of electrogenesis on multiple TTXs-Navs without abrogating proteins per se, providing specific functional interference. A complete block of Nav1.7 activity is not intended since it may induce a state of total insensitivity to pain where unintended self-injury would occur (64).

Pain-sensing PSNs can become hyperexcitable in response to peripheral nerve injury, which in turn leads to the development of neuropathic pain. Multiple lines of evidence from both preclinical and clinical studies demonstrate that block of peripheral nociceptive input can effectively relieve pain symptoms including spontaneous pain (65, 66). Therefore, treatments targeting the peripheral PSNs both avoid CNS side effects and also are likely to succeed. Indeed, a recent expert commentary states that “activity in primary afferent neurons represents a ‘low-hanging target’ in the development of safe therapies” for patients with chronic pain (56). Delivering drugs to the DRG is well developed and safe, for instance as used by anesthesiologists for regional blockade and by pain physicians for diagnosis and treatment of radiculopathy (67). Injection into the DRG has minimal consequences in preclinical models (68). It has also been demonstrated that unintentional intraganglionic injection commonly accompanies clinical transforaminal epidural steroid injection (67), a very common procedure with minimal risk of nerve damage. Thus, the PSNs are particularly suitable for targeting new analgesic treatments, especially at the levels of associated pathological DRG (59, 69). A recent study reports that central nervous system gene therapy by intravenous high-dose AAV causes asymptomatic and self-limited DRG inflammation and mild PSN degeneration in primates (70). Since these changes are very minor in comparison to the those induced by painful and neuropathic conditions that AAV injection would treat, this is unlikely to become a barrier to the clinical application of our approach.

In preclinical models, direct DRG delivery of AAVs encoding analgesic biologics can provide relief in chronic pain, with high transduction efficiency, flexibility for selective segmental localization, and minimal behavior changes attributable to the surgical procedure (71). In parallel, injection techniques are being advanced to achieve minimal invasive delivery of biologics for future clinical pain therapy (72, 73). Small peptides derived from the target protein sequences can serve as decoy molecules to selectively interfere with the function of their target signaling proteins by preemptively binding to them (13). We have successfully employed this strategy in rat models to induce analgesia by block of T-type/Cav3.2 channel functions (12) and by blocking membrane trafficking of Cav2.2 channels via interrupting its interactions with the structural protein of collapsin response mediator protein 2 (CRMP2) (13). Here, we extend the applicability of DRG-AAV strategy to the analgesic effectiveness of multiple PSN TTXs Navs blockade for neuropathic pain. These encouraging results that indicate efficacy and tolerability, if further validated for long-term efficacy and minimal side-effects, suggest the transformational potential of the approach for developing addiction free peripheral pain therapeutic agents. Beyond peripheral nerve injury-induced pain, dysfunctional Navs have been found in various pain conditions, such as osteoarthritis (OA) that is frequently highlighted as an unmet medical need. Thus, for pain conditions like OA, targeting the TTXs Navs might be therapeutically useful (74).

While our studies illustrate the power of rational analgesic peptide drug design strategy and provide encouraging results, we acknowledge several limitations in the current study. Different sodium channels traffic to distinct subcellular locations of PSNs (membrane, terminals, nodes of Ranvier, etc.), and the regulation of this process may provide a number of options to control neuronal excitability in different pathophysiological contexts. Injury-induced peripheral hypersensitization associated with Nav malfunction affects multiple sites of the peripheral sensory nervous system, including augmented pain perception in the peripheral terminals, enhanced nociceptive signal transduction in PSN soma and T-junction, and increased neurotransmission in the spinal dorsal horn. At this early stage, our studies did not investigate differential actions by block of TTXs Navs along the pathway of peripheral nociceptors, nor did the results rule out the possibility that block of TTXs Navs reduces pain by inhibiting afferent hyperexcitable input (75), thus indirectly modulating spinal cord and brain antinociceptive control circuits. Another limitation is that the molecular mechanism(s) of NaviPA1 functioning remains incompletely delineated. Our study has verified lack of pleiotropic effects on BK and calcium channels, but we cannot rule out the possibility of peptide interacting with other unknown targets that mediate protein binding. If the peptide binds to membrane via a lipid mechanism, it might mediate the PM-targeting of a wide array of proteins carrying specialized domains enriched with positive charges. Delineation of the mechanisms in future investigation is critical for the assessment of therapeutic efficacy and potential side effects.

Although we have shown that polybasic NLS and multiple adjacent serine residues are required for NaviPA1 function, phosphorylation-dependent binding of NaviPA1 to the membrane appears unlikely to be essential because serine phosphorylation will neutralize the positive charge of NaviPAI. It has been reported that polybasic peptide with nonphosphorylatable serine shows strong membrane binding (76) and highly polar neutral-serine bearing a hydroxyl group at the terminal carbon offers a stronger interaction with the lipid bilayer membranes (77). Other types of PTMs in the residues of NaviPA1 sequence may also play roles. It is reported that serine PTMs can occur by diverse mechanisms, including phosphorylation, sulfation, acetylation, palmitoylation, myristoylation, and glycosylation (78-80). Different PTMs can alter the charge and hydrophobicity (electrostatics), which in turn induce physicochemical properties, structure, and functional changes of the peptide. Ion channel protein arginine methylation and lysine acetylation can enhance current density by increasing the channel cell surface expression (81, 82). A recent paper reported that alanine substitution of polybasic arginine/lysine in Nav1.7iPA region in Halo-tagged human full-length Nav1.7 does not alter the membrane integration and channel function of Halo-Nav1.7 after transfection (83). It would be interesting to test whether combined mutations of polybasic arginine/lysine and multiple adjacent serine and other conserved residues would change full-length Nav1.7 polar association that will influence channel function. Additionally, the highly disordered NaviPA1 liberated by engineering from full-length Nav1.7 protein likely renders the NaviPA1 different biological properties such as binding to membrane probably via electrostatic interactions and showing an ability for cell nuclear trafficking (12). One possibility that cannot be dismissed is that the nuclear-entry NaviPA1 functions as a transcriptional factor that affects the genes that are critical in regulating Nav1.7 functions, reminiscent of fragmented L-type calcium channel functioning as a transcriptional factor (84, 85). It is also possible that NaviPA1 may function as a decoy peptide that interrupts Nav1.7 interactions with partners, since NaviPA1, which is partially aligned to a putative Nav1.7 dimerization sequence, may affect channel functions by uncoupling Nav1.7 dimerization assembly (86, 87), albeit experimental evidence of such mechanism remains to be shown. The potential signaling pathways that the NaviPAI affected could be many, since Nav1.7 PPI molecule networks involve multiple pathways and Nav1.7 (and other TTXs Navs) intracellular segments serve as essential interfaces for many regulatory signaling molecules, including protein-lipids interactions (35, 36). Alterations of these molecules following nerve injury are essential for ectopic PSN hyperactivity and pain. Future work will address these questions.

### Materials and Methods

### Animals

Adult male and female Sprague Dawley (SD) rats weighing 100-125g body weight (Charles River Laboratories, Wilmington, MA) were used. Animals were housed individually in a room maintained at constant temperature (22±0.5° C.) and relative humidity (60±15%) with an alternating 12 h light-dark cycle and were access to water and food ad libitum throughout the experiment. All efforts were made to minimize suffering, and all survival surgeries were completed in a sterile environment under a surgical microscope in animals anesthetized with isoflurane (2-5%). For tissue harvest euthanasia, animals were deeply anesthetized by isoflurane followed by decapitation with a well-maintained guillotine. The estimated numbers of animals needed were derived from our previous experience with similar experiments (1) and a power analysis was not performed.

The numbers of rats used were detailed in the relevant sections or figure legends of the experiments.

### Computational (in Silico) Designs

Rat full-length Nav1.7 aa sequence was retrieved from the UniProt KB knowledge database (UniProt Knowledgebase release 2018_11). Nav1.7 protein TM domains and intracellular termini and loops were predicted by Phobius (https://www.ebi.ac.uk/Tools/pfa/phobius/) (2). The Nav1.7 protein IDRs were predicted by analyzing the full-length Nav1.7 sequence using DEPICTER (DisorderEd PredictIon CenTER, http://biomine.cs.vcu.edu/servers/DEPICTER/) (3). Potential phosphorylation sites in the Nav1.7 full aa sequence were identified using Disorder Enhanced Phosphorylation Predictor (DEPP, http://www.pondr.com/cgi-bin/depp.cgi) (4). Potentially functional peptides within the IDRs were further analyzed SLIMPrints (http://bioware.ucd.ie/slimprints.html), which predict short linear motifs (SLiMs) based on strongly conserved SLiMs within IDRs (5). Peptide structure determination was analyzed by I-TASSER (https://zhanglab.ccmb.med.umich.edu/I-TASSER/) (6). MacVector ClustalW (MacVector, Apex, NC) was used for vector designs and sequence alignments.

### Molecular Cloning and AAV Constructs

The construct pAAV-CBA-GFP-1.7iPAs encode the GFP-Nav1.7iPA fusion protein downstream a chimeric intron for enhancing transcription, driven by a hybrid CMV enhancer/chicken β-actin (CBA) promoter, and a mRNA stabilizing Woodchuck Posttranscriptional Regulatory Element (WPRE) sequence was inserted downstream of stop code of GFP-Nav1.7iPAs and upstream of human growth hormone poly A signals. Plasmids were subsequently used in transfection experiments and in AAV vector generation. To package AAV6-GFP-1.7iPA1, AAV6-GFPlinker, and AAV2/6-GFP-NP (a Nav1.7 N-terminal inert peptide) (referred to as AAV6-NaviPAI, AAV6-GFP, and AAV6-NP, respectively) for in vivo injection. AAV vectors were produced and purified in our laboratory by previously established methods (7). The titers (GC/mL) of AAV6-GFP), AAV6-NaviPAI, and AAV6-NP vectors were 2.45×10¹³, 3.05×10¹³, 2.64×10¹³, respectively. Same batches of AAVs were used in all in vivo experiments.

Site-directed mutagenesis. Alanine substitution for arginine/lysine and serine on pAAV-CBA-GFPNaviPA1 to generate NaviPAlmt1-6 was made using the QuikChange mutagenesis method (Stratagene). All constructs were verified by DNA sequencing before use.

### Cell Culture

Cell lines. HEK293 cell line stably expressing human wide-type Nav1.7 (HEK1.7) was provided by Dr. Theodore Cummins. HEK293 cell lines stably expressing human wide-type Nav1.6 (HEK1.6), Nav1.3 (HEK1.3), Nav1.1 (HEK1.1), Nav1.5 (HEK1.5), and CHO cells stable expression of human Nav1.8 (CHO1.8) were obtained from Charles River. Neuronal NG108-15 (NG105) neuronal-like cells and F11 cells (hybrid cells of mouse neuroblastoma cells with embryonic rat DRG neurons) were purchased from ATCC (Manassas, VA). Rat DRG-neuronal 50B11 cells (50B11) were used as reported previously (8). These cells were cultured and transfected (PEI 40) using standard techniques, as described previously (1).

Generation of human Nav1.8 stable HEK293 cells. To generate Nav1.8 stable expression HEK293 cells (HEK1.8 cells), an expression plasmid of pcDNA3.1 (+)-SCN10A-Furin-P2A-SCN2B was constructed (Genscript) in which a CMV promoter transcribes human Nav1.8a and Nab2 from a single open reading frame (ORF) expressing human SCN10A (NM_001293306.2) and SCN2B (NM_004588.5) separately linked by a 2A self-processing sequence derived from porcine teschovirus-1 (P2A) and a furin cleavage site (FIG. 12, 13) (9). Final construct was sequence confirmed and transfected in HEK293 cells, and the cells were frequently selected with G-418 (800 μg/mL), followed by establishment of single cell colonies using BIOCHIPS Single-cell Isolation Chip (ThermoFisher) according to the manufacturer recommended protocol. Nav1.8a and Navb2 expression were determined by immunoblots using cell lysates and extracted cell cytosols and membranes, and prioritized by functional sodium current amplitude using conventional whole-cell voltage-clamp (see further).

Dissociated DRG neuronal culture. Dissociated DRG neuronal cultures for EP were performed, as described previously (10) and were studied in 6~8 h after harvest in EP experiments.

Human induced pluripotent stem cells (iPSC)-derived sensory neurons (hiPSC-SNs). HiPSC-SNs, which were derived from female human ectoderm-neural crest stem cells, and ChronoTM Senso-MM complete growth medium were purchased from Anatomic (Minneapolis, MN)

(11). HiPSC-SNs maturing differentiation culture was performed per manufacturers' recommendation.

Generation of Lentivector Expressing NaviPA1 and NP for hiPSC-SNs Transduction

Lentiviral (LV) expression plasmid pWPT-GFP (8) was used to express NaviPA1 and NP (control) (FIG. 14). LV were packaged using pWPT-GFPNaviPA1 and pWPT-GFPNP with packaging plasmid pCMVdR8.74 and envelop plasmid pVSV-g, concentrated, and products titrated in the range of 1×10⁸ to 2×10⁸ transduction unit/mL. Cultured hiPSC-SNs were infected by LV-GFP, GFPNaviPA1 or LV-GFPNP in the presence of 8 mg of polybrene (Sigma-Aldrich) per mL at an optimized multiplicity of infection ≈5.

### Electrophysiology (EP)

EP recordings were performed, as we described previously with minor modifications at room temperature (22~25° C.) (10, 12), in a blind manner where the electrophysiologist was not aware of the treatment. Patch pipettes 0.9-2.5M2 resistance were formed from borosilicate glass (King Precision Glass Co., Claremont, CA) and fire polished. Recordings were made with an Axopatch 700B amplifier (Molecular Devices, Downingtown, PA). Signals were filtered at 5 kHz and sampled at 20 and 50 kHz (adopted from literature) (13-22) with a Digidata 1440A digitizer and pClamp10 software (Molecular Devices, San Jose, CA). Series resistance (3-5M (2) was monitored before and after the recordings, and data were discarded if the resistance changed by 20%. After achieving the whole-cell recording, capacitance (Cm) and series resistance (Rs) were compensated accordingly.

Sodium channel current (I_{Na}) recording in cultured cell lines. Whole-cell voltage-clamp to recording I_{Na} was performed in HEK1.7, HEK1.1, HEK1.3, HEK1.6, HEK1.5, HEK1.8, CHO1.8, NG108-15 cells, and F11cells in current-density (I-V) and fast-inactivation voltage protocols. External solution consists of the following (in mM): 110 NaCl, 20 tetraethylammonium-Cl, 0.01 CaCl₂), 0.1 CaCl2, 5 MgCl2, 10 HEPES and 5.56 mM glucose (pH 7.4, 310-315 mosM/L). The internal pipette solution consisted of (in mM): 10 NaCl, 130 CsCl, 5 MgCl2, 5 EGTA, 2.5 Na2+ATP and 10 HEPES (pH 7.2). After formation of a tight seal (maximal leak amplitude <150 pA), membrane resistance and capacitance were determined. The voltage dependence of activation was assessed from holding potential using 50 ms pulses (test-pulse) to a range of test potentials from −100 mV to +50 mV in 5 mV or 10 mV incremental steps with an interval of 5 s. Current density was calculated by normalizing maximal peak currents with cell capacitance. The voltage dependence of steady-state fast inactivation was measured using a two-step protocol. A 500 ms pre-pulse with various potentials ranging from Vhold to +10 mV in 5 or 10 mV incremental steps was used to inactivate the channels. This pre-pulse was immediately followed by a 40 ms test-pulse at 0 mV to determine the remaining fraction of available channels. Inward current measured during the test-pulse to 0 mV was normalized to the cell's maximum test-pulse inward current. To determine the conductance-voltage (I-V) relationships of voltage-dependent activation, the peak current densities during each voltage command step were fitted to a smooth curve with a Boltzmann equation: I=Gₘₐₓ (V-Eᵣₑᵥ)/[(1+exp [(V−V₅₀)/k))], which provided the maximum conductance (Gₘₐₓ). Normalized activation curves were fitted with a Boltzmann equation G/Gₘₐₓ=1/(1+exp (V50-Vm)/k), where G was calculated as follows: G=I/(Vm−Erev). The steady-state inactivation curves were fitted with I/Imax=1/(1+exp−(V₅₀-Vm)/k). In all the equations, V₅₀ denotes the half-activation and half inactivation potentials, Vm is the membrane potential, Erev is the reversal potential, k is the slope factor, G is the conductance, and I is the current at a given Vm; Gₘₐₓ and Imax are the maximum conductance and current, respectively. Current density was obtained by dividing the maximum peak current (pA) by the cell capacitance (pF). Voltage errors defined by Rs×Imax were minimized by using 80-85% series resistance compensation, and the compensation was readjusted before each voltage-clamp protocol. Cells were excluded if the voltage error exceeded 5 mV.

TTXs and TTXr I_{Na} recording in DRG dissociated neurons (male rats) and hiPSC-SNs. Isolated I_{Na} was recorded from single small/medium DRG neurons (≤35 mm in diameter, 4wk after AAV-DRG injection in naïve rats) and differentiated hiPSC-SNs in bath solution that contained the following (in mM): 80 NaCl, 50 choline-Cl, 30 TEA-C1, 2 CaCl2, 0.2 CdC12, 10 HEPES, and 5 glucose, pH 7.3 with NaOH. Internal solution containing the following (in mM): 70 CsCl, 30 NaCl, 30 TEA-CI, 10 EGTA, 1 CaCl2, 2 MgCl2, 2 Na2ATP, 0.05 GTP, 10 HEPES, and 5 glucose, pH 7.3 with CsOH. A voltage protocol was adopted to separate TTXr I_{Na} and TTXs I_{Na} (14, 23). In brief, A 500 ms prepulse to −120 or −50 mV was applied before a 50 ms test pulse from −100 to 40 mV with steps of 5 or 10 mV by test pulses from −50 to 0 mV. Both TTXs and TTXr I_{Na} were apparent after the −120 mV prepulse; only TTX-R I_{Na} was obtained after the −50 mV prepulse, and the TTXs component was obtained by digitally subtracting the TTXr I_{Na} from the total I_{Na}. EP recording was performed in differentiated hiPSC-SNs with extensive neurite growth at DIV21-28. To isolate somatic I_{Na}, a brief prepulse to voltage (−40 mV) near spikes inactivating hiPSC-SN axonal spikes but not somatic spikes was performed, as described previously (24).

High-voltage activated (HVA) I_{Ca} and Voltage-gated potassium channel current (I_{Kv}). To record HVA I_{Ca} was recorded in rat DRG dissociated neurons (sham-operated, AAV6-GFP, AAV6-NP, and AAV6-NaviPA1 transduced neurons, 4wk after AAV-DRG injection), and BK I_{Kv} recording were conducted in non-differentiated NG108-15 cells, as described previously (1).

Whole-cell current-clamp recording on dissociated DRG neurons (male rats). Whole-cell current-clamp recording of dissociated DRG neurons was performed, as described previously (10, 25, 26). Dissociated small- and medium-sized DRG neurons (≤40 μm in diameter) from sham-operated animals, rats with TNI only, and dissociated DRG neurons with clear GFP expression from TNI rats injected with AAV6-GFPNP or AAV6-NaViPA1 at 8-week after TNI and 6-week after vector injection were used for recording (n=5 rats per group). The membrane input resistance was calculated by dividing the ending amplitude of steady-state hyperpolarizing voltage deflection by the injected current (27). APs were generated by injection of a series of current pulses (180 to 280 pA in steps of 20 pA, 250 ms). The baseline potentials were recorded for 20 ms before the stimulus pulses were injected into the neurons. Resting membrane potential (RMP) was defined as the mean value of the 20 ms pre-stimulus potential in the first trial and the AP rheobase as the minimum current required to evoke the first AP. The neurons with stable resting membrane potentials (RMP) more negative than-40 mV and overshooting APs (>80 mV RMP to peak) were used for additional data collection. AP frequency was determined by quantifying the number of APs elicited in response to depolarizing current injections (250 ms).

Microinjection of AAV Vectors into DRG

AAV vector solution was microinjected into right L4 and L5 DRG using previously described techniques (1, 28). Rats received L4 and L5 DRG injections of either AAV6-NaviPA1 or AAV6-NP (one vector per rat), consisting of 2 μL with adjusted titers containing a total of 2.0×10¹⁰ genome viral particles. Sham in animals denotes that no injection after exposure surgery.

### Animal Pain Model and Behavior Testing

TNI. Animals were anesthetized using isoflurane at 4% for induction and 2% for maintenance. TNI surgery was performed as we described previously (1). Sham-operated rats were subjected to all preceding procedures without nerve ligation and transection.

Stimulated behavior testing. Behavioral tests were conducted between 9:00 AM and 12:00 AM, as we described previously (1). The experimenters were blinded to the treatment during all data acquisition. Stimuli were applied to the lateral margin of the plantar aspect of the foot in the sural area of innervation. Sensory tests included eliciting reflexive behaviors induced by von Frey test, Pin test, cold stimulation (acetone), and heat stimulation (Hargreaves test), and were carried out as previously described (28).

Gabapentin injection. Gabapentin (GBP, Sigma-Aldrich) was dissolved in saline immediately before injections and administered intraperitoneally (i.p.) at a volume of 0.5-1.0 ml (final dose=100 mg/kg body weight).

Conditioned place preference (CPP). A 3-chamber CPP apparatus was used (Med Associates, St. Albans, VT) in which 2 sliding doors separate the central chamber from the 2 side chambers that have distinct wall stripes and flooring. The CPP procedure: 1) On day 1, rats were acclimated to the CPP boxes for 30 min, with open access to each of the three chambers. On the preconditioning day, rats were placed in the grey middle chamber and allowed to explore both sides of chambers for 15 minutes and the time spent in each side was recorded, and the preferred and nonpreferred chambers were identified. 2) On the conditioning days, place conditioning was conducted using a biased assignment approach to drug pairing: saline was paired with the preferred chamber in the morning, and GBP was paired with the non-preferred chamber in the afternoon with a 6 hr interval (injections were never paired with the middle grey chamber). Conditioning consisted of the following sequential steps: intraperitoneal injection and restriction of the animal within the preferred chamber (saline) or non-preferred chamber (GBP) for 45 min. We used a 45 min conditioning time based on tests that gabapentin maximally reduced mechanical hypersensitivity at 30-60 min after i.p. injection (1). Animals were conditioned for 2 days since 2-day GBP has been reported sufficient to produce CPP in rodent pain models (29, 30); 3) For postconditioning testing, the animals were placed back into the middle grey chamber of the CPP chambers with free access to all chambers for 15 minutes. The difference score for each animal was calculated, by subtracting the time spent in the saline-paired or GBP-paired chamber before pairing (during preconditioning) from the time spent in each chamber after pairing (postconditioning), and then averaged within each group. Each rat had only a single CPP test six weeks after AAV injection. A CPP is defined if the animals spend significantly more time in the GBP-paired chamber versus the saline-paired compartment.

### Immunofluorescent Staining

The previously described protocol was adopted (8). Primary antibodies: mouse GFP (1:500, Santa Cruz Biotechnology, SCB, CA. sc9996), rabbit GFP (1:500, Cell signaling, Danvers, MA. 2555), rabbit Nav1.7a (1:400, Alomone, ASC008), rabbit Nav1.6a (1:400, Alomone, ASC009), rabbit glial fibrillary acidic protein (GFAP, 1:1000, Dako, CA, Z0334), goat myelin basic protein (MBP, 1:500, SCB, sc13912), mouse neurofilament (NF200, 1:1000, Sigma-Aldrich, N6389), and mouse b3Tubulin (Tubb3, 1:500, SCB, sc-80016). The fluorophore-conjugated (Alexa 488 or Alexa 594, 1:2000) secondary antibodies (Jackson ImmunoResearch, West Grove, PA) were used to reveal immune complexes. The immunostaining was examined, and images were captured using a Nikon TE2000-S fluorescence microscope (El Segundo, CA) with filters suitable for selectively detecting the green and red fluorescence using a QuantiFire digital camera (Optronics, Ontario, NY). For measurement and quantification of immunostaining, positive marker antibody immunostainings were defined as the cells with the fluorescence intensity greater than average background fluorescence plus 2 standard deviations of the cells in an adjacent area in the same IHC slide of negative control (the first antibody omitted) under identical acquisition parameters (n=10 for different markers). NIH ImageJ software (http://rsbweb.nih.gov/ij/) was used for analysis.

### Immunoblot

Immunoblots were performed as described previously (1). To examine the subcellular localization of Nav1.7 in HEK1.7 cells and NaviPA1 and NaviPA1 mutants in HEK1.7 cells, the cell pellets were homogenized and then fractionated to obtain the plasma membrane, cytosolic, and nuclear fractions were extracted using the ProteoExtract Subcellular Proteome Extraction Kit (Millipore, Billerica, MA). In some experiments, the transferred PVDF membranes were cut into two halves along protein size around 70-100 kDa and were subsequently incubated overnight at 4° C. with appropriate antibodies. Antibodies: mouse GFP (1:1000), rabbit Nav1.7a (1:1000), rabbit Nav1.8a (1:1000, Alomone, ASC-016), rabbit Navb2 (1:1000, Alomone, ASC-007), mouse Na+/K+ATPasela (NKAla, 1:600, SCB, sc514614), mouse Lamin B1 (1:1000, Proteintech, 66095), and mouse Gapdh (1:5000, Sigma-Aldrich, SAB1403850). Immunoreactive proteins were detected by Pierce enhanced chemiluminescence (ThermoFisher) on a ChemiDoc Imaging system (Bio-Rad) after incubation for 1 hr with HRP-conjugated second antibodies (1:5000, Bio-Rad).

### GFPNaᵥiPA1 Affinity Pull-Down Followed by Immunoblots, Silver Staining, Mass Spectrometry, and PIP Strip Assay

GFP affinity pull-down. This was performed using ChromoTek GFP-Trap kit. Briefly, HEK1.7 cells were transiently transfected to express GFPNaviPA1 or GFPlinker and/or GFP1.7iPA2 as controls. After 48 h, cells were lysed with ice-cold nondenaturing lysis buffer containing (in mM): 10 Tris/Cl pH 7.5, 150 NaCl, 0.5 EDTA, with 0.5% Nonidet P40 substitute (a non-ionic and non-denaturing detergent), and protease inhibitor cocktail. The extracted cell lysates were diluted with 300 mL of dilution buffer containing (in mM) 10 Tris/Cl pH 7.5, 150 NaCl, 0.5 EDTA. For GFP-affinity pulldown, GFP-Trap Agarose Beads were equilibrated by adding 25 mL to 1.5 mL reaction tube with 500 mL of dilution buffer, cell lysates (2.5 mg total protein per sample) were incubated with GFP-Trap agarose by tube rotated end-over-end overnight at 4° C., followed by precipitation of beads by centrifugation at 12,000 rpm.

Silver stain, Nav1.7 immunoblot, and mass spectrometry. The extracted cell lysates (input) and GFP-affinity pulldown beads (pulldown) were performed for 4-20% SDS-PAGE gel, followed by immunoblots using Nav1.7 antibody and silver stain on an additional SDS-PAGE gel using Pierce silver stain kit (ThermoFisher). The stained gel regions of interests were excised, and in-gel trypsin digested, as described previously (31). Extracted tryptic peptides were analyzed by nano reversed-phase liquid chromatography tandem mass spectrometry (nLC-MS/MS) using a nanoACQUITY (Waters Corporation, Milford, MA) online coupled with an Orbitrap Velos Pro hybrid ion trap mass spectrometer (ThermoFisher). The precursor ions were selected automatically by the instrument. Resultant MS/MS data were analyzed using the Mascot search engine (Matrix Science version 2.4) against the SWISSPROT human database.

PIP strip assay. NG108-15 cells transfected with GFPNaviPA1 and GFPlinker were prepared for cell lysate using a denaturing RIPA buffer containing (in mM): 10 Tris-HCl pH 7.5, 150 NaCl, 0.5 EDTA, with 0.1% SDS, 1% Triton X100, 1% deoxycholate, and protease inhibitor cocktail. The GFP-affinity pulldown beads from NG108-15 cells were size separated using 4-20% SDS-PAGE gels followed by silver stain. PIP strip assay (Echelon Biosciences, Salt Lake City, UT) was performed to analyze interactions between purified GFPNaviPA1 from NG108-15 cells and membrane lipids, compared to purified GFP from NG108 as control. In brief, PIP (phosphatidylinositol phosphate) strip membranes were blocked in 3% (w/v) fatty acid-free BSA (Sigma-Aldrich) in TBST (50 mM Tris-HCl, pH 7.5, 150 mM NaCl, and 0.1% Tween 20) for 1 h. The membranes were then incubated in the same solution with the purified GFPNaviPA1 or GFP (equal 1.5 mg/mL) overnight at 4° C. with gentle agitation. The membranes were washed 3 times over 30 min in fatty acid-free BSA-TBST. The membranes were incubated for 1h with 1:2000 dilution of HRP conjugated anti-GFP monoclonal antibody (Proteintech, HRP66002) at room temperature, then washed 6 times over 1 h in TBST, and the protein that was bound to the membrane by interaction with phospholipids was detected by enhanced chemiluminescence on a ChemiDoc Imaging system. PIP2 lipid spots: LPA, Lysophosphatidic acid; LPC, Lysophosphocholine; PtdIns, Phosphatidylinositol; PtdIns (3) P, Phosphatidylinositol (3) phosphate; PtdIns (4) P, Phosphatidylinositol (4) phosphate; PtdIns (5) P, Phosphatidylinositol (5) phosphate; PE Phosphatidylethanolamine; PC, Phosphatidylcholine; SIP, Sphingosine 1-Phosphate; PtdIns (3,4)P₂, Phosphatidylinositol (3,4) bisphosphate; PtdIns (3,5)P₂, Phosphatidylinositol (3,5) bisphosphate; PtdIns (4,5)P₂, Phosphatidylinositol (4,5) bisphosphate; PtdIns (3,4,5) P3, Phosphatidylinositol (3,4,5) trisphosphate; PA, Phosphatidic acid; PS, Phosphatidylserine.

### Example 2—Mechanism of Dual Block of Nav1.7/1.8 Channels by a Nav Inhibitory Peptide Aptamer

Multiple sensory neuronal sodium channels (Navs) contribute to pain pathogenesis, indicating that development of a strategy for specific block of multiple Navs, e.g., Nav1.7 and Nav1.8 (Nav1.7/1.8), in the pathological sensory neurons would be more effective for inhibiting pain signals than blocking only single Nav subtype. Nav proteins are enriched with intrinsically disordered regions (IDRs) in their intracellular segments, which can be the key hubs in Nav signaling networks and new drug targets. We discovered a polyacidic peptide from Nav1.7-IDRs in its intracellular loop 2, and voltage-clamp recording on Nav1.7 and Nav1.8 stably expressing HEK cells (HEK1.7 and HEK1.8) shows that this peptide potently inhibits sodium currents (I_{Na}) conducted by both Nav1.7 and Nav1.8. This dual Nav1.7/1.8 inhibitory peptide aptamer (named 1.7/1.8iPA) contains a conserved Ankyrin (AnkG) protein binding domain and a multi-PDZ-domain protein class I binding domain (Pdzd2-I) between Nav1.7 and Nav1.8, suggesting that either AnkG or Pdzd2-I domains or both within 1.7/1.8iPA be responsible for the dual Nav1.7/1.8 inhibition.

The Overall goal of this exploratory/innovative proposal is to investigate and determine whether AnkG and/or Pdzd2-I domains within 1.7/1.8iPA contribute to its dual Nav1.7/1.8 inhibition and whether expression of sequence-defined 1.7/1.8iPA in the primary sensory neurons (PSN) will suppress PSN excitability, thus supporting 1.7/1.8iPA as a potential analgesic lead. Experiments are designed to test: 1) Ion channel selectivity of 1.7/1.8iPA, including a) Effects on various Navs in HEK cells stably expressing Nav subtypes, b) Role of AnkG and/or Pdzd2-I domains in the 1.7/1.8iPA for dual inhibition of Nav1.7/1.8, c) 1.7/1.8iPA effects on Kv7.2/7.3 (both containing conserved AnkG binding domain), and d) Defining the key 1.7/1.8iPA sequence. 2) Validation of dual inhibition of 1.7/1.8iPA on both TTXs and TTXr I_{Na}, effects on Kv7.2/7.3 channels, and on PSN excitability in rat PSNs. As an initial and conceptual stages of the project development, the study will test the hypothesis that 1.7/1.8iPA elicits dual block of Nav1.7/1.8 via a combined effect of AnkG and Pdzd2-I domains, which will suppress PSN action potential firing. Two Specific Aims are proposed to investigate whether 1.7/1.8iPA can be developed as a potential lead for AAV-mediated, sensory neuron-specific analgesia for future study.

### Aim 1. In Vitro (Cell Lines) Investigation of Ion Channel Selectivity of 1.7/1.8iPA

Exp. 1A. Determine the impact of 1.7/1.8iPA on endogenous Nav1.7 and Nav1.8 intracellular trafficking. This will test the hypothesis that 1.7/1.8iPA reduces Nav1.7 and Nav1.8 channel conductance (HEK1.7 and HEK1.8 cells) by decreased protein density in the membrane due to impeded Nav1.7 and Nav1.8 intracellular trafficking.

Exp. 1B. Investigate the specificity of Nav occupancy by voltage-clamp after transfection of 1.7/1.8iPA into HEK1.7, 1.8, 1.6, 1.5, 1.1, and 1.3 cells, as well as NG108-15 neuronal cells that naturally express potassium channels Kv7.2/7.3.

Exp. 1C. Determine the roles of AnkG and/or Pdzd2-I domains in 1.7/1.8iPA action. a) Site-directed mutagenesis of key amino acids (aa) and/or deletion of AnkG or Pdzd2-I domains (or both) within1.7/1.8iPA, coupled with voltage-clamp recording. b) The key aa sequence within 1.7/1.8iPA that are critical for inhibition of Nav1.7/1.8 channels with least effect on Kv7.2/7.3 will be defined and cloned into an AAV plasmid for the studies in Aim 2.

Exp. 1D. Confirm dual Nav1.7/1.8 inhibition by defined 1.7/1.8iPAto assure replicability and compare to the effects on Nav1.8 and Nav1.9 expressed in ND7/23 cells.

Aim 2. Determination of biological activity of defined 1.7/1.8iPA expression in sensory neurons

Exp. 2A. Produce AAVs carrying the sequence-defined 1.7/1.8iPA (d1.7/1.8iPA) and 1.7NP (a non-functional peptide from N-terminus of Nav1.7 as control) for AAV-mediated d1.7/1.8iPA expression in rat PSNs.

Exp. 2B. Determine PSN-specific expression of d1.7/1.8iPA on PSN electrogenesis and toxicity, compared to control. Examine d1.7/1.8iPA on a) TTXs and TTXr Navs conductance, b) Kv7.2/7.3 activity, c) PSN action potential (AP) firing, and d) Neurotoxicity by evaluation of imaging live PSN neurite outgrowth.

Exp. 2C. Replicate TTXs and TTXr I_{Na} recording on PSNs expressing d1.7/1.8iPA in Dr. Cummins's lab.

Exp. 2D. Evaluate behaviors and safety. Determine effects of PSN-specific expression of d1.7/1.8iPA in vivo on a) Normal sensory behavioral thresholds in rats and b) DRG and afferent axonal safety by immunohistochemistry.

### BACKGROUND

The peripheral sensory neuronal Navs are ideal targets for controlling pain. Navs are key regulators of neuronal excitability. I-4 Mammals possess nine isoforms of Navs, of which Nav1.7, Nav1.8, and Nav1.9 are preferably expressed in the PSNs of dorsal root ganglia (DRG). Navs are key determinants regulating action potential generation and propagation, so restraining Navs function in PSNs can have profound effects on limiting neuronal excitability and pain signaling.^{5,6} Nav1.7 remains a leading target among Navs for developing therapies to treat chronic pain, but the success in development of clinical Nav1.7-specific inhibitor as analgesics has been limited.^{3,7} Accumulating evidence demonstrates that multiple PSN-Nav alterations in various pain pathologies are responsible for pain pathogenesis,⁸ indicating that multifaceted block of several nociceptive Navs selective in PSNs would have an analgesic advantage, compared to just block of single Nav subtype.⁹⁻¹²

Small molecules vs biologics. Concerns over drug safety related to poor Nav subtype specificity and low efficacy have limited the translation of systemically administered small molecular compounds into the clinic.^{13,14} Development of biologics targeting Nav1.7 is attractive for analgesia¹⁵⁻¹⁷. Nav1.7 neutralizing monoclonal antibodies were reported to have analgesic efficacy, but the results have not been replicated.¹⁸ Tarantula peptide Nav1.7 blockers^{17,19} have analgesic effectiveness but have poor membrane permeability, inadequate Nav1.7 selectivity, and short half-lives.¹³ Nav1.7-RNAi13 and CRISPR-dCas9 or ZEN epigenetic Nav1.7 suppression²⁰ for analgesic gene therapy have been proposed, but these interventions at the mRNA and epigenetic levels lack the specificity of direct channel intervention, reducing safety and permitting off-target effects,^{13,21,22} and anti-dCas9 immunity creates additional challenge for CRISPR gene therapies.²³ Our previous studies illustrate that AAV-mediated, PSN-specific expression of small analgesic peptides to inhibit nociceptive ion channel activity can provide effective pain relief in multiple animal pain models.²⁴⁻²⁶ Intravenous high-dose AAV causes minimal and self-limited DRG inflammation and mild PSN degeneration in primates.²⁷ Since these changes are very minor in comparison to the changes induced by painful neuropathic conditions that AAV-targeted DRG therapy would treat, this is an unlikely barrier to translation of AAV-DRG analgesic approach in neuropathic pain.

Targeting intrinsically disordered regions (IDRs) facilitates discovery of ion channel inhibitory peptide. Small peptides derived from pronociceptive ion channels as functionally interfering peptide aptamers (iPA) are highly effective and selective, allowing block of specific nociceptive signaling.^{25,28} IDRs of ion channel proteins are commonly engaged in promiscuous interactomes, which are important players in multiple signaling regulations and are considered as new and promising drug targets. 6.29-31 Nociceptive ion channel proteins are enriched with IDRs in their intracellular non-structured segments which can be the key hubs in channel nociceptive signaling networks.²⁵ We have succeeded in identification of disordered Cav3.2iPA for adeno-associated virus (AAV)-mediated, sensory neuron-targeted analgesia.^{24,25} Here, by targeting IDRs, we discovered a disordered polyacidic inhibitory peptide (named 1.7/1.8iPA, 38mer) from the Nav1.7 intracellular loop 2 (ICL2), and voltage-clamp recording showed that this peptide potently inhibits I_{Na} conducted by both Nav1.7 and 1.8 channels (Aim 1. Preliminary data). Further validation of selectivity of 1.7/1.8iPA on ionic channel occupancy and on sensory neuronal excitability will provide insights to decide whether 1.7/1.8iPA could be a potential analgesic lead to be developed for AAV-mediated, PSN-specific analgesia.

B. INNOVATION. 1. The experiments detailed in Example 2 pursues a novel approach by targeting IDRs of Navs to discover a dual Nav1.7/1.8 peptide antagonist. 2. The designs of the analgesic therapeutic peptides directly from Nav1.7 protein are entirely novel.

### C. Approach

Overall Plan: Experiments will test 1.7/1.8iPA on channel protein intracellular trafficking, INaS, and I_{Kv}7.2/7.3 in vitro using HEK Nav subtype stable cells and NG10⁸ cells (or Kv7.2/7.3 stable expression cell lines) (Aim 1); as well as the effects of defined 1.7/1.8iPA expression in rat PSNs on TTXs and TTXr Navs, PSN excitability, normal sensory behaviors, and histological safety (Aim 2).

### Specific Aim 1. In Vitro (Cell Lines) Investigation of Ion Channel Selectivity of 1.7/1.8iPA

Rationale: 1.7/1.8iPA (38mer) contains conserved AnkG and Pdzd2-I binding domains, suggesting that either AnkG or Pdzd2-I domain (or both) within the 1.7/1.8iPA may be responsible for its dual Nav1.7 and Nav1.8 inhibition. Mutagenesis of key aa within either AnkG or Pdzd2-I binding domains (or both), coupled with voltage-clamp, will be performed to evaluate the roles of these domains for dual Nav1.7/1.8 inhibition. Additionally, functional AnkG binding domains exist in multiple Navs and Kv7.2/7.3, thus, vigorous investigation of 1.7/1.8iPA on various pronociceptive Navs and antinociceptive Kv7.2/7.3 is required.

### Preliminary Data for Specific Aim 1

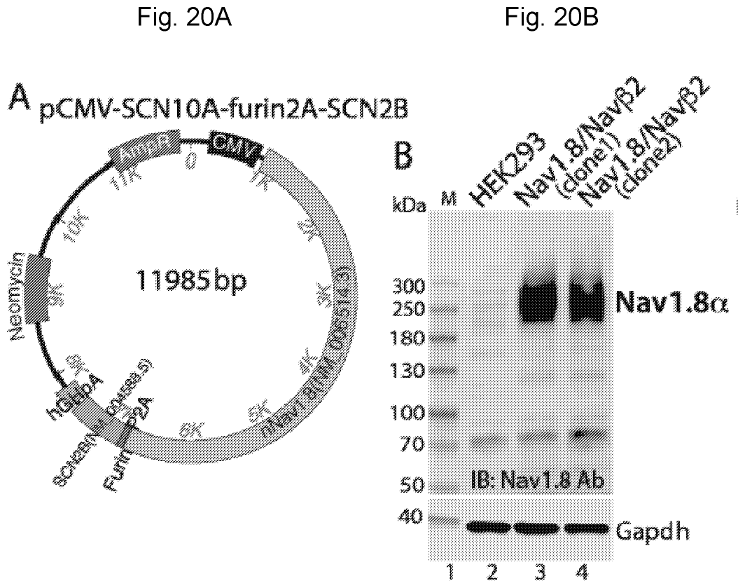

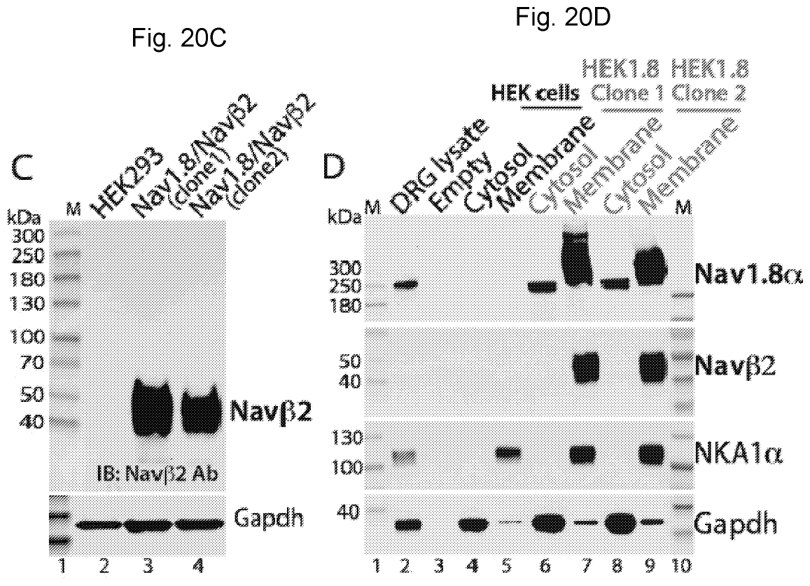

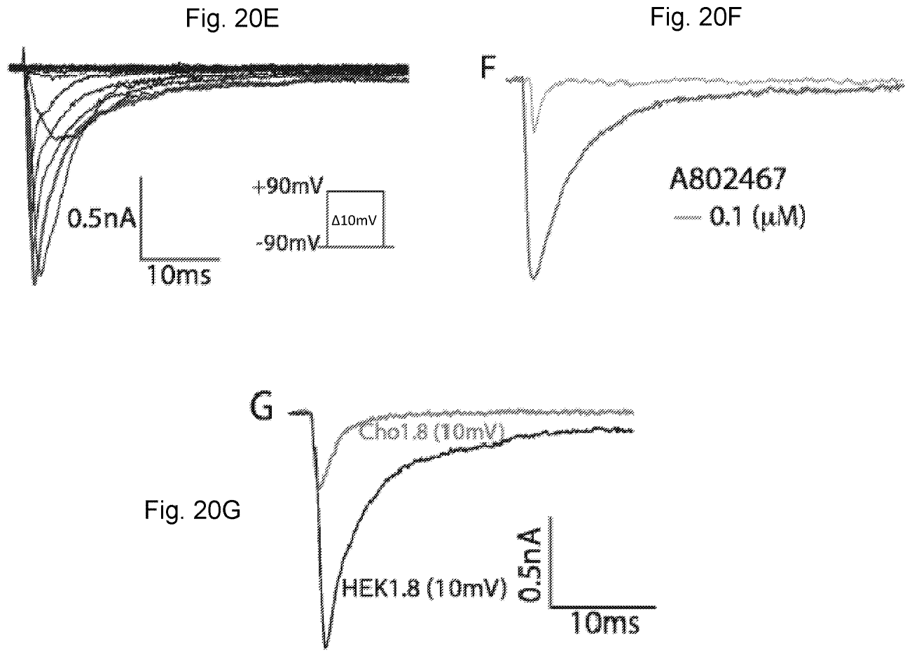

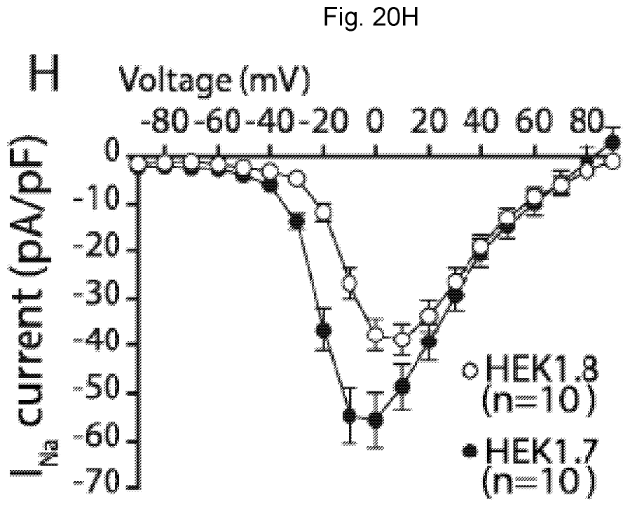

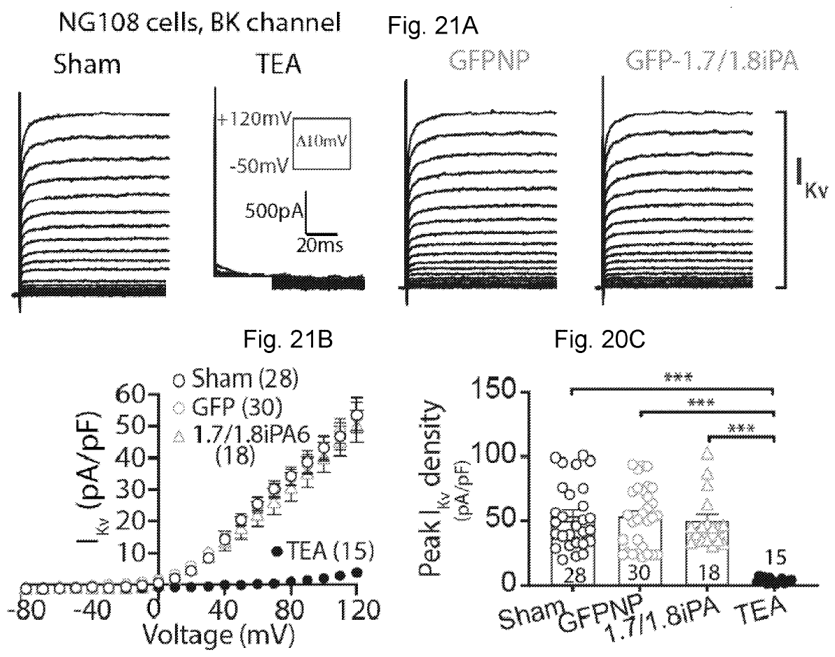

Establishment of Nav1.8 stable expression system based on HEK cells. Currently, commercial HEK1.8 stable cells with sufficient I_{Na1.8} are unavailable. We therefore developed stable and functional expression of recombinant human Nav1.8 heterologous systems in HEK cells. An expression plasmid of pcDNA3.1 (+)-SCN10A-Furin-P2A-SCN2B was constructed in which a CMV promoter transcribes human Nav1.8a and Nab2 from a single open reading frame (ORF) expressing human SCN10A (NM_001293306.2) and SCN2B (NM_004588.5) separately linked by a 2A self-processing sequence derived from porcine teschovirus-1 (P2A) and a furin cleavage site.³² Final construct was transfected into HEK cells, and the cells were frequently selected with G-418 (400~800 μg/mL), followed by establishment of single cell colonies using BIOCHIPS Single-cell Isolation Chip. Stable Nav1.8a/Nab2 expression in HEK cells, highly integrated in the cell membranes, was confirmed by immunoblots of Nav1.8a and Nab2. Functional Nav1.8 expressing clonal cell lines were identified by the presence of slowly inactivating inward I_{Na} elicited by a voltage steps from −140 mV to +40 mV on whole-cell voltage-clamp recordings and the averaged peak I_{Na1.8} density in 85% of the cells was ~1.0 nA, and was sensitive to a Nav1.8 channel blocker, A803467 (85% I_{Na1.8} inhibition with 100 nM of A803467) 33 (FIG. 20).

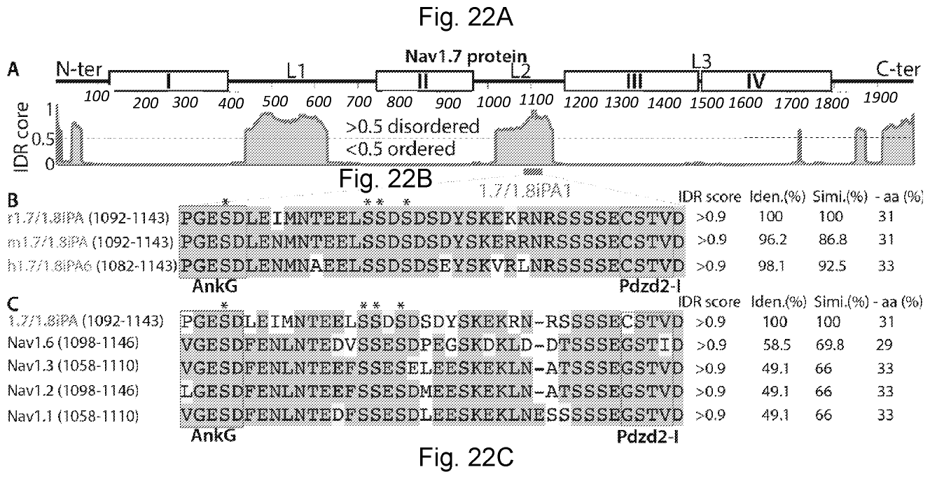

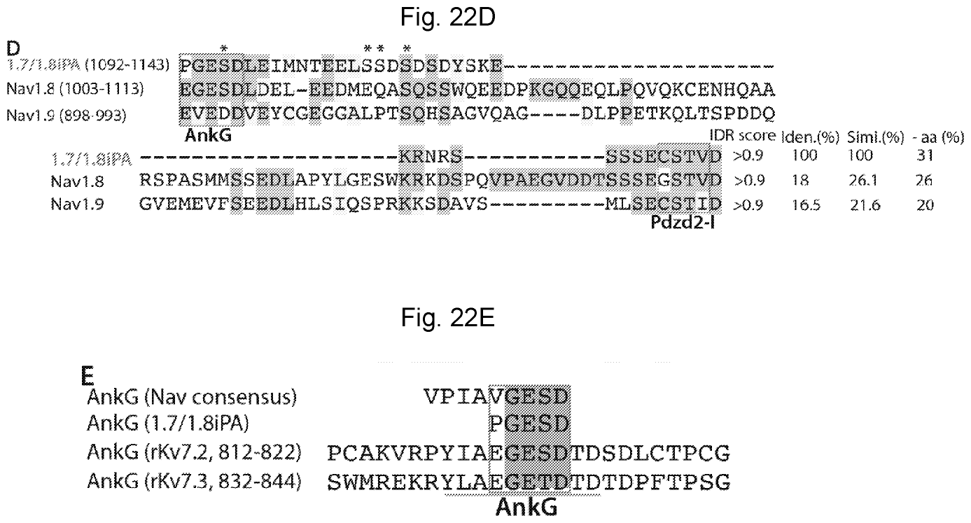

Targeting IDRs of Nav1.7 facilitates discovery of a dual Nav1.7/1.8 inhibitory peptide. We discovered a disordered peptide from the Nav1.7-ICL2. This highly disordered polyacidic peptide is composed of 35% electrically negative-charged glutamic (E) and aspartic (D) acids. Voltage-clamp recording (FIG. 21) shows that presence of this 38mer peptide in HEK1.7 cells inhibits I_{Na1.7} and possibly desensitized Nav1.7 voltage-gated activation; and serendipitously, it also suppresses I_{Na1.8} after expression in HEK1.8 cells. While these need further validation, we refer to it as 1.7/1.8 inhibitory peptide aptamer (1.7/1.8iPA). No change of Big Potassium (BK) I_{Kv} was noted after 1.7/1.8iPA expression in NG10⁸ cells that naturally expression various Kvs. However, BK channel may not contain Kv7.2/7.3 components.³⁴ Further sequence alignment between 1.7/1.8iPA and the corresponding sequence from Nav1.8-ICL2 reveals no significant homology; however, two functional domains, the AnkG binging and Pdzd2-I binding domains,^{35,36} are conserved, suggesting that either AnkG or Pdzd2-I domain or both within 1.7/1.8iPA may contribute to the dual Nav1.7/1.8 inhibition. The AnkG binding domain is also conserved in Kv7.2/7.3 (FIG. 22). It is well-known that AnkG expression is enriched at electrogenic sites in the nervous system, e.g. the axonal initial segment (AIS) and nodes of Ranvier.^{37,38} Pdzd2 binds directly to Nav1.8 and Nav1.7 via three Pdzd2 domains (class I, class IIx2, and class III) in the ICL2 of Nav1.7/1.8, and silencing Pdzd2 in PSNs reduces I_{Na1.8}.³⁵

Neurons target high densities of voltage-gated Nav and Kv channels to their AISs and nodes of Ranvier, governing the initiation and conduction of fast action potentials, respectively.^{37,38} Nav AnkG and Pdzd2 binding domains are known to interact with AnkG and Pdzd2 proteins, respectively, that modulate Nav intracellular trafficking, functional expression, channel properties, and neuronal excitability.^{35,39,40} Kv7.2/7.3 control neuronal excitability and participate in antinociception electrogenesis.⁴¹ Both Kv7.2/7.3 share a conserved domain in the C-termini for binding to AnkG protein,^{39,42} resulting in increasing surface expression of Kv7.2/Kv7.3 to the AIS and nodes of Ranvier. Thus, dual I_{Na1.7/1.8} inhibition by 1.7/1.8iPA may be via interrupting the interactions of Nav1.7/1.8 with AnkG and/or Pdzd2 proteins, which would result in suppression of neuronal excitability. On the other hand, suppressing effects on neuronal excitability by 1.7/1.8iPA might be compromised if the 1.7/1.8iPA had dominant inhibitory effects on Kv7.2/7.3. It has been reported that silencing AnkG in neurons inhibits inward Navs and reduces outward Kv to less of an extent, while neuronal excitability is suppressed,^{38,43,44} AnkG over-expression has slight effects on Kv7.2/7.3 channel gating,³⁹ and Pdzd2 ablation in neurons reduce I_{Na1.8}.³⁵ Thus, it is conceivable that convergent effects of 1.7/1.8iPA expression in PSNs will suppress PSN excitability. We will directly test AnkG and Pdzd2-I domains of 1.7/1.8iPA on INaS, IKV7.2/7.3, and PSN action potential generation.

### Experimental Designs and Analytic Plan for Aim 1

Exp. 1A. Determine 1.7/1.8iPA on endogenous Nav1.7 and Nav1.8 intracellular trafficking: This will test the hypothesis that 1.7/1.8iPA might reduce Nav1.7 and Nav1.8 channel conductance by decreased channel density in the membrane due to impeded Nav1.7 and Nav1.8 intracellular trafficking. Immunoblots of 1.7/1.8iPA (GFP fused, ~35 kDa) and endogenous Nav1.7 and Nav1.8 (~250 kDa), using 1.7NP and sham-transfection as controls, will be performed in parallel on total lysate and the fractionized membrane, cytosol, and nuclear fractions prepared from 1.7/1.8iPA transfected HEK1.7 and HEK1.8 cells. The ratio of target protein bands in total lysates, and fractionized membrane, cytosol, and nuclear samples will be quantified by densitometry.

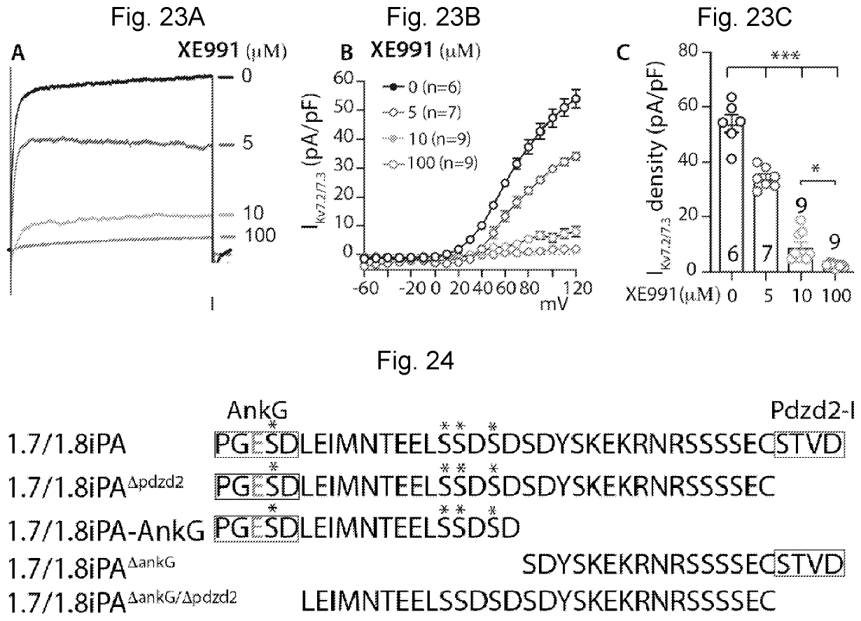

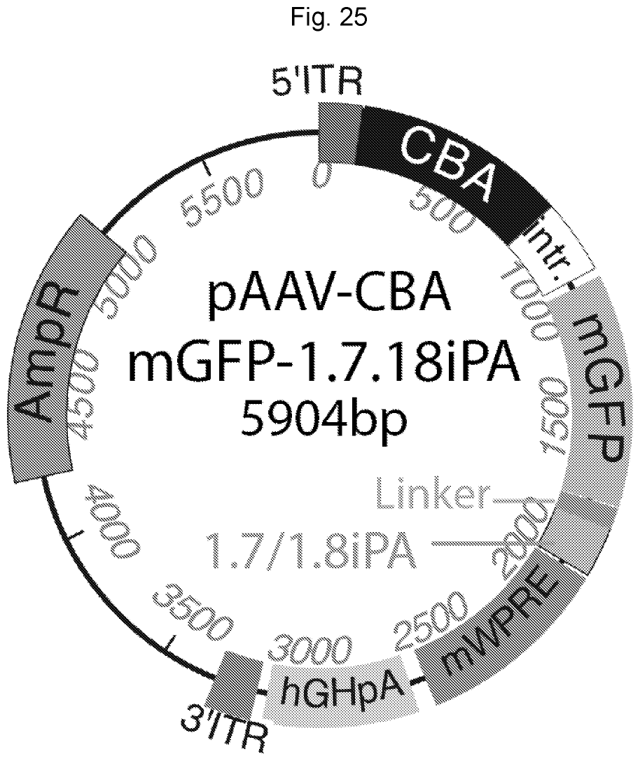

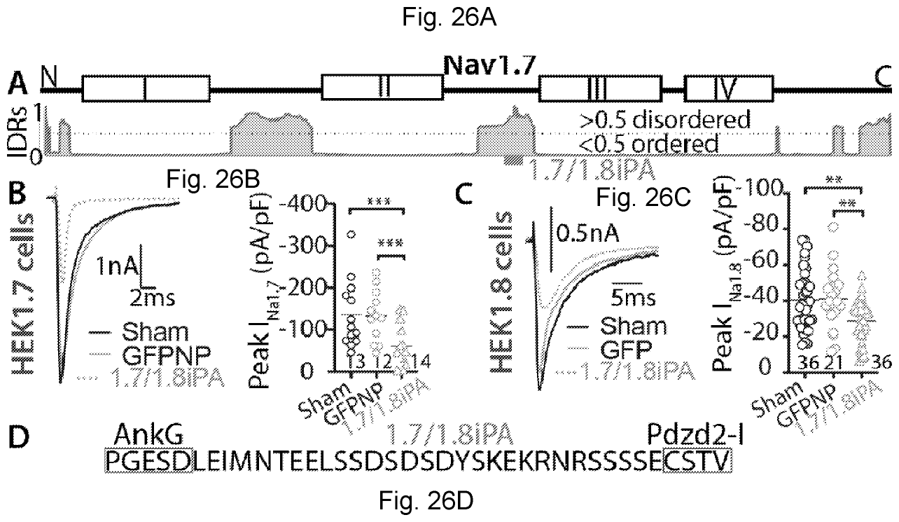

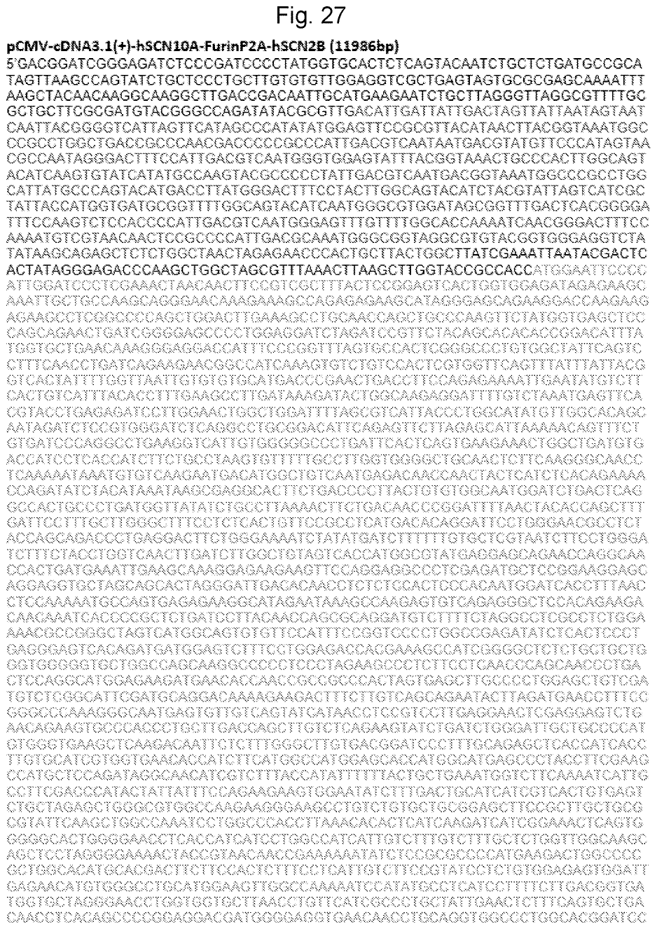

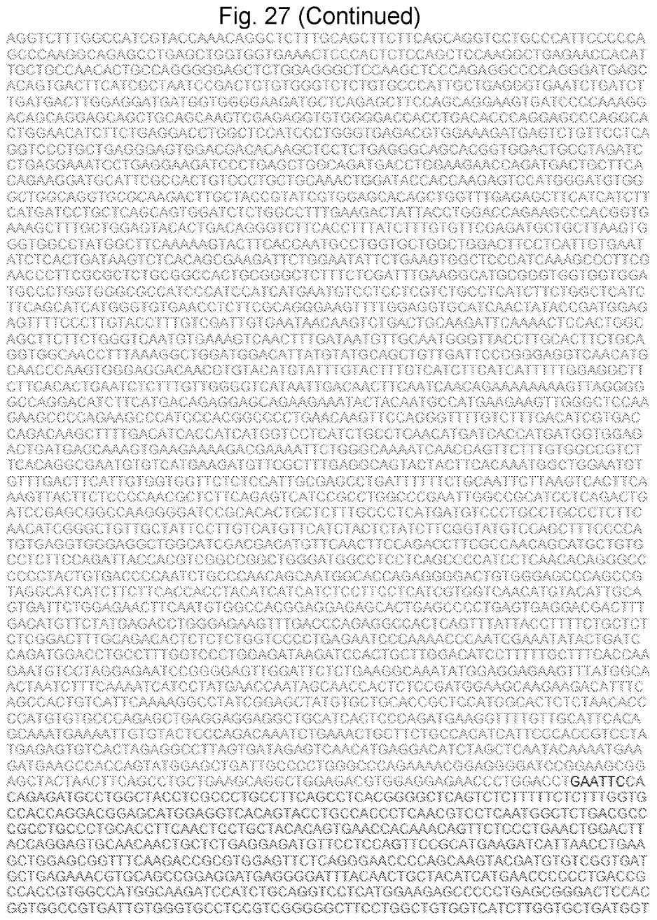

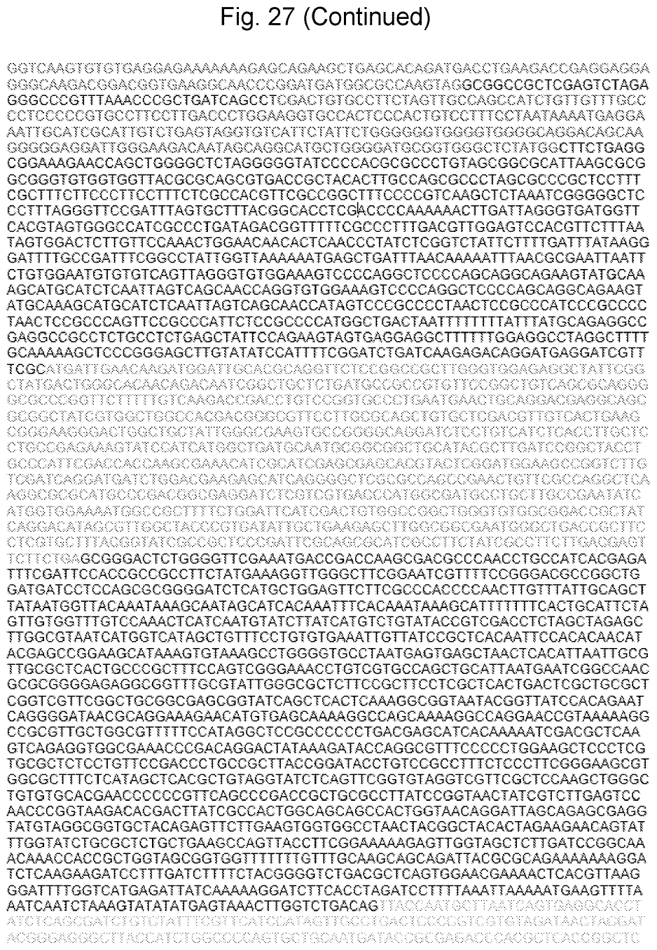

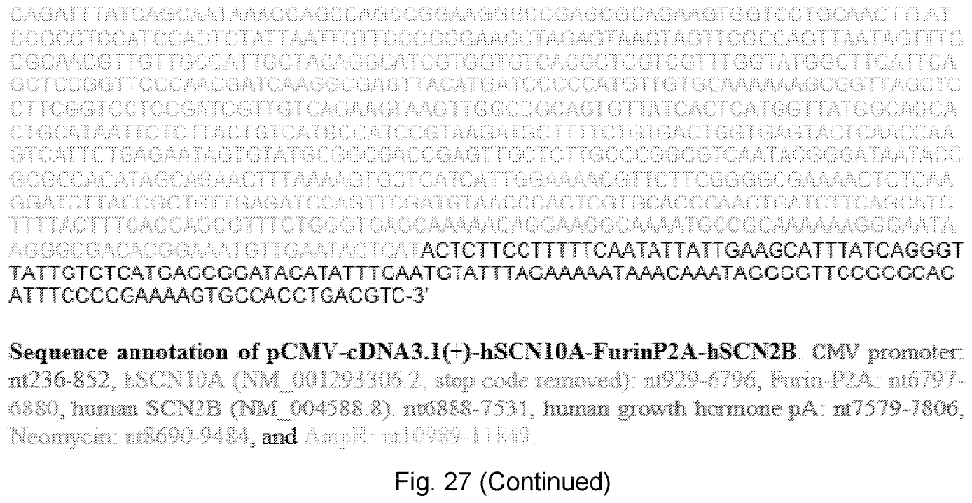

Exp. 1B. Investigate the specificity of Nav and K₁7.2/7.3 channel occupancy (INaS and I_{Kv7.2/7.3}). It is reported that AnkG or Pdzd2 binding domains exist in all Navs.³⁹ In the experimental design, 1) 1.7/1.8iPA effects on the I_{Na} of Nav1.7, Nav1.8, Nav1.3, Nav1.1, Nav1.5, and Nav1.6 in HEK Nav-subtype stable cells (available in Yu lab) will be quantitatively analyzed (peak I_{Na} density and gating properties) by whole-cell voltage-clamp, using 1.7NP and sham-transfection as controls; 2) 1.7/1.8iPA effects on I_{Kv7.2/7.3} (M-current) will be recorded and quantitatively analyzed in NG108-15 (NG108) neuronal cells that naturally express Kv7.2/7.3.^{45,46} We have succeeded in recording M-current in NG108 cells (FIG. 23) using the protocol adopted as published;^{41,47,48} with XE991, a K7.2/7.3 selective inhibitor.⁴⁹ Alternatively, HEK cells stable expression of human Kv7.2/Kv7.3 channels (HEK-Kv7.2/7.3) will be obtained from Charles River (Cat #CT6147); and 3) If I_{Kv7.2/7.3} were inhibited, immunoblots will determine Kv7.2/7.3 membrane integration, as described in Exp. 1A, using Kv7.2/7.3 specific antibodies and NG10⁸ cells or HEK-Kv7.2/7.3 cells.

Exp. 1C. Determine the roles of AnkG and/or Pdzd2-I domains in 1.7/1.8iPA. Site-directed mutagenesis of key aa and/or deletion of AnkG or Pdzd2-I domains or both will be performed, coupled with voltage-clamp as described in Exp. 1B, to record Navs and Kv7.2/7.3 (based on the results of Exp. 1B) and immunoblots will be performed to detect channel membrane integration (as described in Exp. 1A). The Key aa sequence of 1.7/1.8iPA that are critical for inhibition of Nav1.7/1.8 with least effects on Kv7.2/7.3 will be defined and cloned into a AAV shuttle plasmid to produce AAV for the studies in Aim 2. The glutamate (E) and nearby casein kinase (CK2) phosphorylation serine residues(S) have been defined as the key sites for Nav-AnkG binding.⁵⁰

Exp. 1D. Validation. Confirm dual Nav1.7/1.8 inhibition in HEK1.7, HEK1.8, and ND7/23 Nav1.8 cells^{51,52} by d1.7/1.8iPA and test d1.7/1.8iPA against an optimized human Nav1.9 construct that functionally expresses in ND7/23 cells.

### Analytic Plan: Milestones and Criteria for Judging Success of Aim 1

Immunoblotting determination and quantitative analysis whether presence 1.7/1.8iPA will impede intracellular trafficking of endogenous Nav1.7 and Nav1.8 in HEK1.7 and HEK1.8 cells.

Electrophysiological (EP) determination of the selectivity of 1.7/1.8iPA on Nav1.7, Nav1.8, Nav1.1, Nav1.3, Nav1.6 and cardiac Nav1.5 using HEK Nav-subtype stable cells. Negative controls have no effect (≤5%) on current inhibition of these Nav isoforms. Results are validated and Nav1.9 is tested in Dr Cummins's lab.

EP determination whether presence 1.7/1.8iPA will affect Kv7.2/7.3 conductance, and if so, whether Kv7.2/7.3 intracellular trafficking will be impeded, using NG108 or HEKKv7.2/7.3 cells.

Success in mapping and defining the key 1.7/1.8iPA aa sequence for Nav1.7/1.8 inhibition (≥50) with least effects on Kv7.2/7.3 conduction (≤20%).

Statistics: p<0.05 for significance, unpaired, two-tailed student's t test; one-way ANOVA and Tukey post hoc, where appropriate. Sampling power calculation where appropriate and data will be analyzed in collaboration with the statistical consultant (Department of Biostatistics, MCW) on a basis of fee for service (FFS).²⁵

Validation of Dual Nav1.7/1.8 Inhibition with Comparison to Nav1.9.

Aim 1 will be completed, and timelines and milestones achieved within the 1st grant year (GY).

Expected Results, Potential Problems and Alternative Strategies for Aim 1.1.) 1.7/1.8iPA inhibits peak I_{Na1.7} and 1.8 densities with possible shifts of voltage-gated activation (FIG. 21). However, these results need to exclude the possible voltage-clamp errors, defined by compensated Rs×Imax, that is proportionally greater for the large I_{Na} in the control cells than the markedly inhibited I_{Na} in the cells expressing 1.7/1.8iPA. To overcome this, we will characterize Nav1.7 gating kinetics in presence of 1.7/1.8iPA by reduction of bath Na⁺ concentration to 50 mM.^{53,54} 2.) We anticipate that AnkG binding domain within 1.7/1.8iPA might be dominant over Pdzd2-I, as Nav1.7 and Nav1.8 share three Pdzd2 binding domains, i.e., class I, class IIx2, and class III within the ICL2. Thus, it is expected that the sequence of AnkG domain with CK2 phosphorylation sites⁵⁰ would be defined as the key sequence for 1.7/1.8iPA in dual block of Nav1.7/1.8. 3.) Multipronged Nav inhibition. The d1.7/1.8iPA might also be able of inhibiting other conserved Navs, especially the TTXs Nav1.6, 1.3, and 1.1, due to sequence homology. Since our goal is to develop AAV-mediated, PSN-specific expression of the d1.7/1.8iPA for multi-pronociceptive Nav inhibition, spillover blockade of other Navs restricted in PSNs may be advantageous by AAV-delivered selectively to DRG.^{55,56 4}.) A novel Nav1.9 optimized cDNA is available and will be used to test the effects of d1.7/1.8iPA on Nav1.9. And if necessary, I_{Na1.9} can be tested using dissociated PSNs from Nav^{1.8} knockout mice.⁵⁷

### Specific Aim 2. Determine Biological Activity of Defined 1.7/1.8iPA Expression in Sensory Neurons

1) Does d1.7/1.8iPA inhibit I_{Na} conducted by TTXs and TTXr, and also I_{Kv} conducted by Kv7.2/7.3 in PSNs? 2) More importantly, will PSN excitability be suppressed (or enhanced) by d1.7/1.8iPA expression?

### Preliminary Data, Experimental Designs, and Analytic Plan for Aim 2

Exp. 2A. Produce high quality of AAVs. Produce high quality of AAV carrying the d1.7/1.8iPA and the control NP (FIG. 25) for AAV-mediated d1.7/1.8iPA expression in rat PSNs by DRG injection.⁵⁹

Exp. 2B. Determine impact of PSN-specific expression of d1.7/1.8iPA on PSN electrogenesis and biological activity. AAV will be injected into lumbar (L) 4/5 DRG of adult rats by our established techniques.^{25,59,60} DRG will be harvested 4wk post AAV injection, DRG dissociated culture will then be established, and d1.7/1.8iPA and NP (both GFP-fused) expressing neurons will be identified under the fluorescent microscope for the following the studies: a) Whole-cell voltage-clamp recording to determine expression of d1.7/1.8iPA on TTXs and TTXr I_{Na} in PSNs⁶¹⁻⁶³; b) Effects of d1.7/1.8iPA on PSN-Kv7.2/7.3 channel activity, compared to control (NP); c) Whole-cell current-clamp recording to analyze d1.7/1.8iPA effects on AP firing of PSNs;^{24,25} and d) Determine neurotoxicity of d1.7/1.8iPA expression by imaging live PSN neurite outgrowth, which can be used for evaluating neurotoxicity.⁶⁴

Exp. 2C. Replicate recordings of TTXs and TTXr I_{Na} and AP firing on d1.7/1.8 expressing PSNs. Viral vectors for infecting dissociated DRG neurons will also be provided.

Exp. 2D. Evaluate behaviors and safety (MCW lab). Evaluate PSN-specific expression of d1.7/1.8iPA on normal sensory thresholds in rats. This will be cooperated with Exp. 2B. Specifically, after baseline sensory behaviors (mechanical: von Frey and Pin and thermal: Heat and Cold) are tested, AAVs (AAV-d1.7/1.8iPA and NP control) will be injected into L4/5 DRG, and behaviors will be evaluated on a weekly basis for 4 wk (10 rats per group) as we described previously,^{25,65} followed by DRG dissociated culture for EP studies and DRG/Sciatic nerve IHC. Statistics will be analyzed to compare sensory thresholds between AAV-d1.7/1.8iPA and NP control.⁶⁵

Analytic Plan. Milestones Criteria for Judging Success of Aim 2

Production of AAV6-GFPd1.7/1.8iPA and AAV6-GFPNP for in vivo studies. a) AAV titer ≥1×10¹³ GC/ml, and b) high purity (1:1:10 ratios of AAV virion capsid protein Vp1: Vp2: Vp3 that comprise ≥90% of total silver-stained proteins on 1d SDS-PAGE gel).

TTXs and TTXr I_{Na} in PSNs. Determination of d1.7/1.8iPA expression in block of both TTXs and TTXr Navs (inhibition of TTXs and TTXr I_{Na}≥50%). No significant effects of NP on Navs.

Kv7.2/7.3 in PSNs. Investigation of inhibitory effects of d1.7/1.8iPA expression of PSN I_{KV7.2/7.3}. Our prediction is that this will be ≤20% by d1.7/1.8iPA, compared to control.

PSN excitability. Determine the impact of d1.7/1.8iPA expression on AP firing of PSNs.

Replication. Replication of dual inhibition of TTXs and TTXr I_{Na} and AP in Dr. Cummins's lab.

Neurotoxicity. Live-cell imaging of PSN neurite outgrowth in culture validates minimal neurotoxicity of PSNs expressing d1.7/1.8iPA, compared to control.

Sensory behaviors. Determination the effects of AAV-mediated, PSN-specific expression of d1.7/1.8iPA on normal sensory thresholds in rats. We predict minimal alteration of normal sensory thresholds.

IHC determines DRG d1.7/1.8iPA expression, PSN neurotoxicity, and axonal neuropathy.

Statistics: p<0.05 for significance, unpaired two-tailed student's t test; one-way and two-way ANOVA followed by Tukey post hoc, where appropriate. Sampling power calculation where needed and data analysis will be in collaboration with our statistical consultant (MCW) on FFS.²⁵

Experiments in Aim 2 are completed, and milestones achieved in the 2^{nd} GY.

Expected Results, Potential Problems, and Alternative Strategies for Aim 2. 1.) The inventors have substantial experience with DRG-targeted analgesia using AAVs in rodent models and EP recording of various ion channels in PSNs and PSN excitability,^{13,14,16,40-42} so they do not expect any technological challenges. 2.) It has been reported that silencing AnkG in neurons reduces inward Navs and also outward Kv in less extent while neuronal excitability is suppressed.^{38,43,44} Pdzd2 ablation in PSNs reduces I_{Na1.8}.³⁵ Thus, we expect that d1.7/1.8iPA expression in PSNs will suppress AP firing of PSNs, supporting the potential of d1.7/1.8iPA as an analgesic. 3.) Inhibitory effects of d1.7/1.8iPA on channels are expected via interrupting interaction (PPI) between channels and AnkG and/or pdzd2 protein in PSNs (not in the scope of this proposal due to R21 budget limitation). Proof of concept as described in 2.) will facilitate targeting relevant PPI pathways in our future studies. 4.) Safety concerns. Unlike knockout strategy that ablates the production of a target protein by which the irreversible silencing of AnkG gene causes pathological consequences, e.g., axonal pathology and neurological dysfunction;⁶⁶ the inventors anticipate that d1.7/1.8iPA will be non-toxic, since AAV-mediated d1.7/1.8iPA expression selectively in PSNs should provide sustained and restricted blockade of Nav1.7/1.8 without abrogating proteins per se, resulting in specific functional interference. 5.) AAV production. Production of high quality AAV for in vivo DRG delivery is well-established in our lab, so we do not expect challenges for this. We are using the AAV6 serotype due to our findings of efficient gene transfer to the full range of DRG-PSNs including the nociceptive sub-populations, without significant glial transduction.^{25,26,60,67 6}) Although elevated pain sensitivity is described in females compared to males, these proof-of-concept experiments will initially focus on males due to budget limitations of the R21. Sex differences will be fully addressed in expanded future studies.

### General Methods

1) Patch-clamp recordings of Navs and Kv7.2/7.3. a) INas and Kv7.2/7.3 in cell lines are recorded at room temperature according to the established protocols. The current density is calculated according to the capacitance of the cell and expressed in pA/picofarad. AP chains are recorded, as we described previously.^{24,25}

3) AAV production⁶⁰: AAV vectors will be prepared by helper-free triple-plasmid transfection of 293T cells. Plasmids include a) AAV expression plasmids; b) pRep2/Cap6 containing AAV2 replication (rep) and capsid (cap) genes from AAV6 (or others); and c) pHelper encoding the adenoviral helper genes.

4) DRG injection^{59,65}: After minimal laminectomy, 2 μl of AAV or controls will be injected into DRG through a pulled smalltip glass micropipette attached to a microprocessor-controlled injector over 5 min. Removal of the pipette will be delayed for an additional 5 min to minimize the extrusion of the injectate, then the wound is closed.

5) Behavioral evaluation^{65,67}: Examination will be performed in a blinded fashion, including a) Brush, b) Cold stimulation, c) von Frey, d) Radiant heat stimulation, and e) Noxious mechanical stimulation (Pin).

6) Statistics: Data will be analyzed with help from our Statistical Consulting Service, Dept. of Biostatistics, MCW. These consulting services include assistance with design and analysis of observational studies, assistance with public databases, sample size and power calculations, and data analysis and interpretation.

### REFERENCES FOR EXAMPLE 2

1 Dib-Hajj, S. D., Cummins, T. R., Black, J. A. & Waxman, S. G. Sodium channels in normal and pathological pain. Annu Rev Neurosci 33, 325-347, doi: 10.1146/annurev-neuro-060909-153234 (2010).
2 Bennett, D. L., Clark, A. J., Huang, J., Waxman, S. G. & Dib-Hajj, S. D. The Role of Voltage-Gated Sodium Channels Pain in Signaling. Physiol Rev 99, 1079-1151, doi: 10.1152/physrev.00052.2017 (2019).
3 Dib-Hajj, S. D., Yang, Y., Black, J. A. & Waxman, S. G. The Na (V) 1.7 sodium channel: from molecule to man. Nat Rev Neurosci 14, 49-62, doi: 10.1038/nrn3404 (2013).
4 Goodwin, G. & McMahon, S. B. The physiological function of different voltage-gated sodium channels in pain. Nat Rev Neurosci 22, 263-274, doi: 10.1038/s41583-021-00444-w (2021).
5 Catterall, W. A. From ionic currents to molecular mechanisms: the structure and function of voltage-gated sodium channels. Neuron 26, 13-25, doi: 10.1016/s0896-6273 (00) 81133-2 (2000).
6 Ovsepian. S. V. & Waxman, S. G. Gene therapy for chronic pain: emerging opportunities in target-rich peripheral nociceptors. Nat Rev Neurosci, doi: 10.1038/s41583-022-00673-7 (2023).
7 Emery, E. C., Luiz, A. P. & Wood. J. N. Nav1.7 and other voltage-gated sodium channels as drug targets for pain relief. Expert Opin Ther Targets 20, 975-983. doi: 10.1517/14728222.2016.1162295 (2016).
8 Rush. A. M., Cummins, T. R. & Waxman, S. G. Multiple sodium channels and their roles in electrogenesis within dorsal root ganglion neurons. J Physiol 579, 1-14, doi: 10.1113/jphysiol.2006.121483 (2007).
9 Eagles, D. A., Chow, C. Y. & King, G. F. Fifteen years of NaV 1.7 channels as an analgesic target: Why has excellent in vitro pharmacology not translated into in vivo analgesic efficacy? Br J Pharmacol, doi: 10.1111/bph. 15327 (2020).
10 King, A. A different path: resh strategies and targets for chronic pain could deliver much-needed replacements for opioid-based painkillers. Nature 573, S4-S6 (2019).
11 McDonnell, A., Collins, S., Ali, Z., Iavarone, L., Surujbally, R., Kirby, S. et al. Efficacy of the Nav1.7 blocker PF-05089771 in a randomised, placebo-controlled, double-blind clinical study in subjects with painful diabetic peripheral neuropathy. Pain 159, 1465-1476, doi: 10.1097/j.pain.0000000000001227 (2018).
12 Siebenga, P., van Amerongen, G., Hay, J. L., McDonnell, A., Gorman, D., Butt, R. et al. Lack of Detection of the Analgesic Properties of PF-05089771, a Selective Na (v) 1.7 Inhibitor, Using a Battery of Pain Models in Healthy Subjects. Clin Transl Sci 13, 318-324, doi: 10.1111/cts.12712 (2020).
13 Kingwell, K. Nav1.7 withholds its pain potential. Nat Rev Drug Discov, doi: 10.1038/d41573-019-00065-0 (2019).
14 Kingwell, K. Navigating a new path to Nav1.7 for pain. Nat Rev Drug Discov 21, 18, doi: 10.1038/d41573-021-00197-2 (2022).
15 Wulff, H. Christophersen, P., Colussi, P., Chandy, K. G. & Yarov-Yarovoy, V. Antibodies and venom peptides: new modalities for ion channels. Nat Rev Drug Discov 18. 339-357, doi: 10.1038/s41573-019-0013-8 (2019).
16 Hatcher, J. P., Chessell, I. P. & Hughes, J. P. Biologics: the next-generation therapeutics for analgesia? Expert Rev Neurother 11, 1653-1658, doi: 10.1586/ern. 11.66 (2011).
17 Shcherbatko, A. Rossi, A., Foletti, D., Zhu, G., Bogin. O., Galindo Casas. M. et al. Engineering Highly Potent and Selective Microproteins against Nav1.7 Sodium Channel for Treatment of Pain. J Biol Chem 291, 13974-13986, doi: 10.1074/jbc.M116.725978 (2016).
18 Lee, J. H., Park, C. K., Chen, G., Han, Q., Xie, R. G., Liu, T. et al. A monoclonal antibody that targets a NaV1.7 channel voltage sensor for pain and itch relief. Cell 157, 1393-1404, doi: 10.1016/j.cell.2014.03.064 (2014).
19 Neff, R. A., Flinspach, M., Gibbs, A., Shih, A. Y., Minassian, N. A., Liu, Y. et al. Comprehensive engineering of the tarantula venom peptide huwentoxin-IV to inhibit the human voltage-gated sodium channel hNav1.7. J Biol Chem 295, 1315-1327, doi: 10.1074/jbc.RA119.011318 (2020).
20 Moreno, A. M., Aleman, F., Catroli, G. F., Hunt, M., Hu, M., Dailamy, A. et al. Long-lasting analgesia via targeted in situ repression of NaV1.7 in mice. Sci Transl Med 13, doi: 10.1126/scitranslmed.aay9056 (2021).
21 Chernikov, I. V., Vlassov, V. V. & Chernolovskaya, E. L. Current Development of siRNA Bioconjugates: From Research to the Clinic. Front Pharmacol 10, 444, doi: 10.3389/fphar.2019.00444 (2019).
22 Setten, R. L., Rossi, J. J. & Han, S. P. The current state and future directions of RNAi-based therapeutics. Nat Rev Drug Discov 18, 421-446, doi: 10.1038/s41573-019-0017-4 (2019).
23 Crudele, J. M. & Chamberlain, J. S. Cas9 immunity creates challenges for CRISPR gene editing therapies. Nat Commun 9, 3497, doi: 10.1038/s41467-018-05843-9 (2018).
24 Itson-Zoske, B., Shin, S. M., Xu, H., Qiu, C., Fan, F., Hogan, Q. H. et al. Selective block of sensory neuronal T-type/Cav3.2 activity mitigates neuropathic pain behavior in a rat model of osteoarthritis pain. Arthritis Res Ther 24, 168, doi: 10.1186/s13075-022-02856-0 (2022).
25 Shin, S. M., Lauzadis, J., Itson-Zoske, B., Cai, Y., Fan, F., Natarajan, G. K. et al. Targeting intrinsically disordered regions facilitates discovery of calcium channels 3.2 inhibitory peptides for adeno-associated virus-mediated peripheral analgesia. Pain 163, 2466-2484. doi: 10.1097/j.pain.0000000000002650 (2022).
26 Yu, H., Shin, S. M., Xiang, H., Chao, D., Cai, Y., Xu, H. et al. AAV-encoded Ca (V) 2.2 peptide aptamer CBD3A6K for primary sensory neuron-targeted treatment of established neuropathic pain. Gene Ther 26, 308-323, doi: 10.1038/s41434-019-0082-7 (2019).
27 Hordeaux, J., Buza, E. L., Dyer, C., Goode, T., Mitchell, T. W., Richman, L. et al. Adeno-Associated Virus-Induced Dorsal Root Ganglion Pathology. Hum Gene Ther 31, 808-818, doi: 10.1089/hum.2020.167 (2020).
28 Yu, H., Shin, S. M., Xiang, H., Chao, D., Cai, Y., Xu. H. et al. AAV-encoded CaV2.2 peptide aptamer CBD3A6K for primary sensory neuron-targeted treatment of established neuropathic pain. Gene Ther, doi: 10.1038/s41434-019-0082-7 (2019).
29 Wright. P. E. & Dyson, H. J. Intrinsically disordered proteins in cellular signalling and regulation. Nat Rev Mol Cell Biol 16. 18-29, doi: 10.1038/nrm3920 (2015).
30 Olah, J., Szenasi, T., Lehotzky, A., Norris, V. & Ovadi, J. Challenges in Discovering Drugs That Target the Protein-Protein Interactions of Disordered Proteins. Int J Mol Sci 23, doi: 10.3390/ijms23031550 (2022).
31 Kjaergaard, M. & Kragelund, B. B. Functions of intrinsic disorder in transmembrane proteins. Cell Mol Life Sci 74, 3205-3224, doi: 10.1007/s00018-017-2562-5 (2017).
32 Fang, J., Yi, S., Simmons, A., Tu, G. H., Nguyen, M., Harding, T. C. et al. An antibody delivery system for regulated expression of therapeutic levels of monoclonal antibodies in vivo. Mol Ther 15, 1153-1159, doi: 10.1038/sj.mt.6300142 (2007).
33 Jarvis, M. F., Honore, P., Shieh, C. C., Chapman, M., Joshi, S., Zhang, X. F. et al. A-803467, a potent and selective Nav1.8 sodium channel blocker, attenuates neuropathic and inflammatory pain in the rat. Proc Natl Acad Sci USA 104, 8520-8525, doi: 10.1073/pnas.0611364104 (2007).
34 Ebner-Bennatan, S., Patrich, E., Peretz, A., Kornilov, P., Tiran, Z., Elson, A. et al. Multifaceted modulation of K+ channels by protein-tyrosine phosphatase epsilon tunes neuronal excitability. J Biol Chem 287, 27614-27628, doi: 10.1074/jbc.M112.342519 (2012).
35 Shao, D., Baker, M. D., Abrahamsen, B., Rugiero, F., Malik-Hall, M., Poon, W. Y. et al. A multi PDZ-domain protein Pdzd2 contributes to functional expression of sensory neuron-specific sodium channel Na (V) 1.8. Mol Cell Neurosci 42, 219-225, doi: 10.1016/j.men.2009.07.003 (2009).
36 Montersino. A., Brachet, A., Ferracci, G., Fache, M. P., Angles d′Ortoli, S., Liu, W. et al. Tetrodotoxin-resistant voltage-gated sodium channel Nav 1.8 constitutively interacts with ankyrin G. J Neurochem 131, 33-41, doi: 10.1111/jnc.12785 (2014).
37 Ho. T. S., Zollinger, D. R., Chang. K. J., Xu, M., Cooper, E. C., Stankewich, M. C. et al. A hierarchy of ankyrin-spectrin complexes clusters sodium channels at nodes of Ranvier. Nat Neurosci 17, 1664-1672. doi: 10.1038/nn.3859 (2014).
38 Zhou, D., Lambert, S., Malen, P. L., Carpenter, S., Boland, L. M. & Bennett, V. AnkyrinG is required for clustering of voltage-gated Na channels at axon initial segments and for normal action potential firing. J Cell Biol 143, 1295-1304, doi: 10.1083/jcb.143.5.1295 (1998).
39 Pan, Z., Kao, T., Horvath, Z., Lemos, J., Sul, J. Y., Cranstoun, S. D. et al. A common ankyrin-G-based mechanism retains KCNQ and NaV channels at electrically active domains of the axon. J Neurosci 26, 2599-2613, doi: 10.1523/JNEUROSCI.4314-05.2006 (2006).
40 Sole, L. & Tamkun, M. M. Trafficking mechanisms underlying Na (v) channel subcellular localization in neurons. Channels (Austin) 14, 1-17, doi: 10.1080/19336950.2019.1700082 (2020).
41 Wang, H. S., Pan. Z., Shi, W., Brown, B. S., Wymore, R. S., Cohen, I. S. et al. KCNQ2 and KCNQ3 potassium channel subunits: molecular correlates of the M-channel. Science 282. 1890-1893, doi: 10.1126/science.282.5395.1890 (1998).
42 King, A. N., Manning. C. F. & Trimmer, J. S. A unique ion channel clustering domain on the axon initial segment of mammalian neurons. J Comp Neurol 522, 2594-2608, doi: 10.1002/cne.23551 (2014).
43 Barry, J., Gu, Y., Jukkola, P., O'Neill, B., Gu, H., Mohler, P. J. et al. Ankyrin-G directly binds to kinesin-1 to transport voltage-gated Na+ channels into axons. Dev Cell 28, 117-131, doi: 10.1016/j.devcel.2013.11.023 (2014).
44 Gasser, A., Ho, T. S., Cheng, X., Chang, K. J., Waxman, S. G., Rasband, M. N. et al. An ankyrinG-binding motif is necessary and sufficient for targeting Nav1.6 sodium channels to axon initial segments and nodes of Ranvier. J Neurosci 32, 7232-7243, doi: 10.1523/JNEUROSCI.5434-11.2012 (2012).
45 Meves, H., Schwarz, J. R. & Wulfsen, I. Separation of M-like current and ERG current in NG108-15 cells. Br J Pharmacol 127, 1213-1223, doi: 10.1038/sj.bjp.0702642 (1999).
46 Rundfeldt, C. The new anticonvulsant retigabine (D-23129) acts as an opener of K+channels in neuronal cells. Eur J Pharmacol 336, 243-249, doi: 10.1016/s0014-2999 (97) 01249-1 (1997).
47 Kay, H. Y., Greene, D. L., Kang, S., Kosenko, A. & Hoshi, N. M-current preservation contributes to anticonvulsant effects of valproic acid. J Clin Invest 125, 3904-3914, doi: 10.1172/JCI79727 (2015).
48 Peretz, A., Degani-Katzav, N., Talmon, M., Danieli, E., Gopin, A., Malka, E. et al. A tale of switched functions: from cyclooxygenase inhibition to M-channel modulation in new diphenylamine derivatives. PLoS One 2, e1332, doi: 10.1371/journal.pone.0001332 (2007).
49 Tsvetkov, D., Tano, J. Y., Kassmann, M., Wang. N., Schubert, R. & Gollasch, M. The Role of DPO-1 and XE991-Sensitive Potassium Channels in Perivascular Adipose Tissue-Mediated Regulation of Vascular Tone. Front Physiol 7, 335, doi: 10.3389/fphys.2016.00335 (2016).
50 Brechet, A. Fache, M. P., Brachet, A. Ferracci, G. Baude, A., Irondelle. M. et al. Protein kinase CK2 contributes to the organization of sodium channels in axonal membranes by regulating their interactions with ankyrin G. J Cell Biol 183, 1101-1114, doi: 10.1083/jcb.200805169 (2008).
51 Xiao, Y., Barbosa, C., Pei, Z., Xie, W., Strong, J. A., Zhang, J. M. et al. Increased Resurgent Sodium Currents in Nav1.8 Contribute to Nociceptive Sensory Neuron Hyperexcitability Associated with Peripheral Neuropathies. J Neurosci 39, 1539-1550, doi: 10.1523/JNEUROSCI.0468-18.2018 (2019).
52 Xiao, Y., Theile, J. W., Zybura, A., Pan, Y., Lin, Z. & Cummins, T. R. A-type FHFs mediate resurgent currentst through TTX-resistant voltage-gated sodium channels. Elife 11. doi: 10.7554/eLife.77558 (2022).
53 Reckziegel, G., Beck. H., Schramm, J., Elger. C. E. & Urban, B. W. Electrophysiological characterization of Na+ currents in acutely isolated human hippocampal dentate granule cells. J Physiol 509 (Pt 1), 139-150, doi: 10.1111/j.1469-7793.1998.139bo.x (1998).
54 Torkkeli, P. H., Sekizawa, S. & French, A. S. Inactivation of voltage-activated Na (+) currents contributes to different adaptation properties of paired mechanosensory neurons. J Neurophysiol 85, 1595-1602, doi: 10.1152/jn.2001.85.4.1595 (2001).
55 Joksimovic, S. L., Joksimovic, S. M., Tesic, V., Garcia-Caballero, A., Feseha, S., Zamponi, G. W. et al. Selective inhibition of CaV3.2 channels reverses hyperexcitability of peripheral nociceptors and alleviates postsurgical pain. Sci Signal 11, doi: 10.1126/scisignal.aao4425 (2018).
56 Na, H. S., Choi, S., Kim, J., Park, J. & Shin, H. S. Attenuated neuropathic pain in Cav3.1 null mice. Mol Cells 25, 242-246 (2008).
57 Akopian, A. N., Souslova, V., England, S., Okuse, K., Ogata, N., Ure, J. et al. The tetrodotoxin-resistant sodium channel SNS has a specialized function in pain pathways. Nat Neurosci 2, 541-548, doi: 10.1038/9195 (1999).
58 Cummins, T. R., Aglieco, F., Renganathan, M., Herzog, R. I., Dib-Hajj, S. D. & Waxman, S. G. Nav1.3 sodium channels: rapid repriming and slow closed-state inactivation display quantitative differences after expression in a mammalian cell line and in spinal sensory neurons. J Neurosci 21, 5952-5961, doi: 10.1523/JNEUROSCI.21-16-05952.2001 (2001).
59 Yu, H., Fischer, G. & Hogan, Q. H. AAV-Mediated Gene Transfer to Dorsal Root Ganglion. Methods Mol Biol 1382, 251-261, doi: 10.1007/978-1-4939-3271-9_18 (2016).
60 Yu, H., Fischer, G., Ferhatovic, L., Fan, F., Light, A. R., Weihrauch. D. et al. Intraganglionic AAV6 results in efficient and long-term gene transfer to peripheral sensory nervous system in adult rats. PLoS One 8, e61266, doi: 10.1371/journal.pone.0061266 (2013).
61 Cummins, T. R., Rush, A. M., Estacion, M., Dib-Hajj, S. D. & Waxman, S. G. Voltage-clamp and current-clamp recordings from mammalian DRG neurons. Nat Protoc 4. 1103-1112. doi: 10.1038/nprot.2009.91 (2009).
62 Cummins, T. R. & Waxman, S. G. Downregulation of tetrodotoxin-resistant sodium currents and upregulation of a rapidly repriming tetrodotoxin-sensitive sodium current in small spinal sensory neurons after nerve injury. J Neurosci 17, 3503-3514 (1997).
63 Lopez-Santiago, L. F., Pertin, M., Morisod, X., Chen, C., Hong, S., Wiley, J. et al. Sodium channel beta2 subunits regulate tetrodotoxin-sensitive sodium channels in small dorsal root ganglion neurons and modulate the response to pain. J Neurosci 26, 7984-7994, doi: 10.1523/JNEUROSCI.2211-06.2006 (2006).
64 Zhang, L., Li. S. & Xia, M. High-Throughput Neurite Outgrowth Assay Using GFP-Labeled iPSC-Derived Neurons. Curr Protoc 2, e542, doi: 10.1002/cpz1.542 (2022).
65 Fischer. G., Kostic, S., Nakai, H., Park, F., Sapunar, D., Yu. H. et al. Direct injection into the dorsal root ganglion: technical. behavioral, and histological observations. J Neurosci Methods 199, 43-55, doi: 10.1016/j.jneumeth.2011.04.021 (2011).
66 Saifetiarova, J., Taylor, A. M. & Bhat, M. A. Early and Late Loss of the Cytoskeletal Scaffolding Protein, Ankyrin G Reveals Its Role in Maturation and Maintenance of Nodes of Ranvier in Myelinated Axons. J Neurosci 37, 2524-2538, doi: 10.1523/JNEUROSCI.2661-16.2017 (2017).
67 Fischer, G., Pan, B., Vilceanu, D., Hogan, Q. H. & Yu, H. Sustained relief of neuropathic pain by AAV-targeted expression of CBD3 peptide in rat dorsal root ganglion. Gene Ther 21, 44-51, doi: 10.1038/gt.2013.56 (2014).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

TABLE 1
Effects of candidate Nav1.7iPAs on I_{Na1.7} and
gating properties of hNav1.7 (HEK1.7 cells).

| | Peak I_{Na1.7} | Activation V₅₀ | Inactivation |
|---|---|---|---|
| Plasmids | (pA/pF) | (mV) | V₅₀ (mV) |
| Sham | 119.9 ± 7.01 (33) | 23.9 ± 1 (33) | 55.7 ± 2.2 (22) |
| TTX (100 ng) | 2 ± 1 (10) | Nd | Nd |
| GFPlinker | 107.5 ± 9.2 (20) | 23.2 ± 1.2 (20) | 56.0 ± 0.8 (15) |
| Nav1.7iPA1 | 43 ± 5 (29)*** | 17.8 ± 0.8 (29)*** | 60.1 ± 1.3 (11) |
| Nav1.7iPA2 | 161.9 ± 12 (23)* | 24.4 ± 1.8 (23) | 70.5 ± 1.8 (16)* |
| Nav1.7iPA3 | 118 ± 1 (22) | 25.4 ± 1.2 (22) | 61.6 ± 2 (18) |
| Nav1.7iPA4 | 49.2 ± 5.4 (20)*** | 15.9 ± 1.6 (20)*** | 54.5 ± 2 (9) |
| Nav1.7iPA5 | 103.5 ± 8.1 (20) | 16.5 ± 1.1 (20)*** | 57.0 ± 2.3 (9) |
| Nav1.7iPA6 | 55.9 ± 6.9 (28)*** | 18.6 ± 1.1 (28)*** | 53.2 ± 1.5 (17) |
| Nav1.7iPA7 | 109.3 ± 7.8 (17) | 24.2 ± 1.9 (17) | 56.3 ± 1.8 (14) |
| Nav1.7iPA8 | 103.3 ± 13.2 (21) | 23.3 ± 1.7 (21) | 61.3 ± 1.8 (23) |
| Nav1.7iPA9 | 83.3 ± 7.8 (13) | 20.3 ± 1.4 (13) | 64.1 ± 5.3 (9) |

Note:
Data are from initial screening results (FIG. 1J).
Nd: not determined.
*, **, and *** denote p < 0.05, 0.01, and 0.001, compared to sham, one-way ANOVA and Turkey post hoc.

TABLE 2
Specificity of Nav1.7iPA1 on I_{Na} and gating properties of hNav isoforms (1.3,
1.5, 1.6, and 1.8) (HEK Nav1.3, 1.5, 1.6, and 1.8 stable cell lines)

| Cells/iPA peptide | Peak I_{Na} (pA/pF) | Activation V50 (mV) | Inactivation V50 (mV) |
|---|---|---|---|
| HEK1.3/Sham | 41.1 ± 7.3 (20) | 20.4 ± 1.8 (20) | 47.6 ± 4.0 (22) |
| HEK1.3/GFPlinker | 49.6 ± 4.7 (21) | 21.4 ± 1.4 (21) | 48.0 ± 3.5 (19) |
| HEK1.3/Nav1.7iPA1 | 24.8 ± 2.2 (26)** | 9.1 ± 1.4 (26)*** | 45.4 ± 4.0 (16) |
| HEK1.5/Sham | 150.3 ± 12.9 (14) | 45.8 ± 1.3 (14) | 76.2 ± 3.7 (13) |
| HEK1.5/GFPlinker | 150.0 ± 13.8 (15) | 42.9 ± 2.8 (15) | 73.8 ± 4.6 (11) |
| HEK1.5/Nav1.7iPA1 | 150.2 ± 18.5 (16) | 42.0 ± 2.5 (16) | 74.4 ± 3.5 (15) |
| HEK1.6/Sham | 119.9 ± 7.1 (30) | 24.0 ± 1.0 (30) | 23.9 ± 1.0 (28) |
| HEK1.6/GFPlinker | 109.9 ± 10.6 (18) | 23.2 ± 2.2 (18) | 23.2 ± 1.2 (17) |
| HEK1.6/Nav1.7iPA1 | 62.7 ± 8.0 (30)*** | 18.9 ± 1.2 (30)*** | 18.6 ± 1.1 (25) |
| HEK1.8/Sham | 40.4 ± 2.8 (36) | 15.0 ± 1.2 (31) | 40.1 ± 4.5 (18) |
| HEK1.8/GFPlinker | 41.2 ± 3.6 (15) | 17.6 ± 1.4 (11) | 42.4 ± 2.8 (16) |
| HEK1.8/Nav1.7iPA1 | 40.7 ± 3.8 (18) | 17.6 ± 1.2 (17) | 42.6 ± 2.8 (31) |

*, **, and *** denote p < 0.05, 0.01, and 0.001, compared to sham, one-way ANOVA and Turkey post hoc.

### REFERENCES

1. Goodwin G, and McMahon S B. The physiological function of different voltage-gated sodium channels in pain. Nat Rev Neurosci. 2021; 22 (5): 263-74.
2. Alsaloum M, Higerd G P, Effraim P R, and Waxman S G. Status of peripheral sodium channel blockers for non-addictive pain treatment. Nat Rev Neurol. 2020; 16 (12): 689-705.
3. Chen L, Huang J, Zhao P, Persson A K, Dib-Hajj F B, Cheng X, et al. Conditional knockout of NaV1.6 in adult mice ameliorates neuropathic pain. Sci Rep. 2018; 8 (1): 3845.
4. Lindia J A, Kohler M G, Martin W J, and Abbadie C. Relationship between sodium channel NaV1.3 expression and neuropathic pain behavior in rats. Pain. 2005; 117 (1-2): 145-53.
5. Bennett D L, Clark A J, Huang J, Waxman S G, and Dib-Hajj S D. The Role of Voltage-Gated Sodium Channels in Pain Signaling. Physiol Rev. 2019; 99 (2): 1079-151.
6. Kingwell K. Nav1.7 withholds its pain potential. Nat Rev Drug Discov. 2019.
7. Wulff H, Christophersen P, Colussi P, Chandy K G, and Yarov-Yarovoy V. Antibodies and venom peptides: new modalities for ion channels. Nat Rev Drug Discov. 2019; 18 (5): 339-57.
8. Hatcher J P, Chessell I P, and Hughes J P. Biologics: the next-generation therapeutics for analgesia? Expert Rev Neurother. 2011; 11 (11): 1653-8.
9 Moreno A M, Aleman F, Catroli G F, Hunt M, Hu M, Dailamy A, et al. Long-lasting analgesia via targeted in situ repression of NaV1.7 in mice. Sci Transl Med. 2021; 13 (584).
10. Setten R L, Rossi J J, and Han S P. The current state and future directions of RNAi-based therapeutics. Nat Rev Drug Discov. 2019; 18 (6): 421-46.
11. Crudele J M, and Chamberlain J S. Cas9 immunity creates challenges for CRISPR gene editing therapies. Nat Commun. 2018; 9 (1): 3497.
12. Shin S M, Lauzadis J, Itson-Zoske B, Cai Y, Fan F, Natarajan G K, et al. Targeting intrinsically disordered regions facilitates discovery of calcium channels 3.2 inhibitory peptides for adeno-associated virus-mediated peripheral analgesia. Pain. 2022; 163 (12): 2466-84.
13. Yu H, Shin S M, Xiang H, Chao D, Cai Y, Xu H, et al. AAV-encoded Ca (V) 2.2 peptide aptamer CBD3A6K for primary sensory neuron-targeted treatment of established neuropathic pain. Gene Ther. 2019; 26 (7-8): 308-23.
14. Wright P E, and Dyson H J. Intrinsically disordered proteins in cellular signalling and regulation. Nat Rev Mol Cell Biol. 2015; 16 (1): 18-29.
15. Eagles D A, Chow C Y, and King G F. Fifteen years of Na (V) 1.7 channels as an analgesic target: Why has excellent in vitro pharmacology not translated into in vivo analgesic efficacy? Br J Pharmacol. 2022; 179 (14): 3592-611.
16. McDonnell A, Collins S, Ali Z, Iavarone L, Surujbally R, Kirby S, et al. Efficacy of the Nav1.7 blocker PF-05089771 in a randomised, placebo-controlled, double-blind clinical study in subjects with painful diabetic peripheral neuropathy. Pain. 2018; 159 (8): 1465-76.
17. Siebenga P, van Amerongen G, Hay J L, McDonnell A, Gorman D, Butt R, et al. Lack of Detection of the Analgesic Properties of PF-05089771, a Selective Na (v) 1.7 Inhibitor, Using a Battery of Pain Models in Healthy Subjects. Clin Transl Sci. 2020; 13 (2): 318-24.
18. Barik A, Katuwawala A, Hanson J, Paliwal K, Zhou Y, and Kurgan L. DEPICTER: Intrinsic Disorder and Disorder Function Prediction Server. J Mol Biol. 2020; 432 (11): 3379-87.
19. Xue B, Dunbrack R L, Williams R W, Dunker A K, and Uversky V N. PONDR-FIT: a meta-predictor of intrinsically disordered amino acids. Biochim Biophys Acta. 2010; 1804 (4): 996-1010.
20. Strzyz P. Disordered interactions. Nat Rev Mol Cell Biol. 2018; 19 (11): 676-7.
21. Zhou J, Oldfield C J, Yan W, Shen B, and Dunker A K. Intrinsically disordered domains: Sequence disorder function relationships. Protein Sci. 2019; 28 (9): 1652-63.
22. Davey N E, Cowan J L, Shields D C, Gibson T J, Coldwell M J, and Edwards R J. SLIMPrints: conservation-based discovery of functional motif fingerprints in intrinsically disordered protein regions. Nucleic Acids Res. 2012; 40 (21): 10628-41.
23. Yu H, Pan B, Weyer A, Wu H E, Meng J, Fischer G, et al. CaMKII Controls Whether Touch Is Painful. J Neurosci. 2015; 35 (42): 14086-102.
24. Roy A, Kucukural A, and Zhang Y. I-TASSER: a unified platform for automated protein structure and function prediction. Nat Protoc. 2010; 5 (4): 725-38.
25. Hornbeck P V, Zhang B, Murray B, Kornhauser J M, Latham V, and Skrzypek E. PhosphoSitePlus, 2014: mutations, PTMs and recalibrations. Nucleic Acids Res. 2015; 43 (Database issue): D512-20.
26. Lin J R, and Hu J. SeqNLS: nuclear localization signal prediction based on frequent pattern mining and linear motif scoring. PLoS One. 2013; 8 (10): e76864.
27. Cummins T R, and Waxman S G. Downregulation of tetrodotoxin-resistant sodium currents and upregulation of a rapidly repriming tetrodotoxin-sensitive sodium current in small spinal sensory neurons after nerve injury. J Neurosci. 1997; 17 (10): 3503-14.
28. Lopez-Santiago L F, Pertin M, Morisod X, Chen C, Hong S, Wiley J, et al. Sodium channel beta2 subunits regulate tetrodotoxin-sensitive sodium channels in small dorsal root ganglion neurons and modulate the response to pain. J Neurosci. 2006; 26 (30): 7984-94.
29. Li R, Walsh P, Truong V, Petersen A, Dutton J R, and Hubel A. Differentiation of Human iPS Cells Into Sensory Neurons Exhibits Developmental Stage-Specific Cryopreservation Challenges. Front Cell Dev Biol. 2021; 9:796960.
30. Milescu L S, Bean B P, and Smith J C. Isolation of somatic Na⁺ currents by selective inactivation of axonal channels with a voltage prepulse. J Neurosci. 2010; 30 (22): 7740-8.
31. Dusan M, Jastrow C, Alyce M M, Yingkai W, Shashikanth M, Andelain E, et al. Differentiation of the 50B11 dorsal ganglion cells into NGF and GDNF responsive nociceptor subtypes. Mol Pain. 2020; 16: 1744806920970368.
32. Li Y, North R Y, Rhines L D, Tatsui C E, Rao G, Edwards D D, et al. DRG Voltage-Gated Sodium Channel 1.7 Is Upregulated in Paclitaxel-Induced Neuropathy in Rats and in Humans with Neuropathic Pain. J Neurosci. 2018; 38 (5): 1124-36.
33. Hendus-Altenburger R, Vogensen J, Pedersen E S, Luchini A, Araya-Secchi R, Bendsoe A H, et al. The intracellular lipid-binding domain of human Na (+)/H (+) exchanger 1 forms a lipid-protein co-structure essential for activity. Commun Biol. 2020; 3 (1): 731.
34. Wang Z Z, Shi X X, Huang G Y, Hao G F, and Yang G F. Fragment-based drug design facilitates selective kinase inhibitor discovery. Trends Pharmacol Sci. 2021; 42 (7): 551-65.
35. Arbesu M, Iruela G, Fuentes H, Teixeira J M C, and Pons M. Intramolecular Fuzzy Interactions Involving Intrinsically Disordered Domains. Front Mol Biosci. 2018; 5:39.
36. Deryusheva E, Nemashkalova E, Galloux M, Richard C A, Eleouet J F, Kovacs D, et al. Does Intrinsic Disorder in Proteins Favor Their Interaction with Lipids? Proteomics. 2019; 19 (6): e1800098.
37. Kaur G, Pinggera A, Ortner N J, Lieb A, Sinnegger-Brauns M J, Yarov-Yarovoy V, et al. A Polybasic Plasma Membrane Binding Motif in the I-II Linker Stabilizes Voltage-gated CaV1.2 Calcium Channel Function. J Biol Chem. 2015; 290 (34): 21086-100.
38. Temmerman K, Ebert A D, Muller H M, Sinning I, Tews I, and Nickel W. A direct role for phosphatidylinositol-4,5-bisphosphate in unconventional secretion of fibroblast growth factor 2. Traffic. 2008; 9 (7): 1204-17.
39. Yamasaki K, Daiho T, Yasuda S, Danko S, Kawabe J I, and Suzuki H. Electrostatic interactions between single arginine and phospholipids modulate physiological properties of sarcoplasmic reticulum Ca (2+)-ATPase. Sci Rep. 2022; 12 (1): 12200.
40. Zeng X, Ruff K M, and Pappu R V. Competing interactions give rise to two-state behavior and switch-like transitions in charge-rich intrinsically disordered proteins. Proc Natl Acad Sci USA. 2022; 119 (19): e2200559119.
41. Dennis M K, Taneva S G, and Cornell R B. The intrinsically disordered nuclear localization signal and phosphorylation segments distinguish the membrane affinity of two cytidylyltransferase isoforms. J Biol Chem. 2011; 286 (14): 12349-60.
42. Maures T J, Su H W, Argetsinger L S, Grinstein S, and Carter-Su C. Phosphorylation controls a dual-function polybasic nuclear localization sequence in the adapter protein SH2B1beta to regulate its cellular function and distribution. J Cell Sci. 2011; 124 (Pt 9): 1542-52.
43. Hammond G R, and Hong Y. Phosphoinositides and Membrane Targeting in Cell Polarity. Cold Spring Harb Perspect Biol. 2018; 10 (2).
44. Itson-Zoske B, Shin S M, Xu H, Qiu C, Fan F, Hogan Q H, et al. Selective block of sensory neuronal T-type/Cav3.2 activity mitigates neuropathic pain behavior in a rat model of osteoarthritis pain. Arthritis Res Ther. 2022; 24 (1): 168.
45. Pan B, Yu H, Fischer G J, Kramer J M, and Hogan Q H. Dorsal Root Ganglionic Field Stimulation Relieves Spontaneous and Induced Neuropathic Pain in Rats. J Pain. 2016; 17 (12): 1349-58.
46. Kingwell K. Navigating a new path to Nav1.7 for pain. Nat Rev Drug Discov. 2022; 21 (1): 18.
47. Shin S M, Wang F, Qiu C, Itson-Zoske B, Hogan Q H, and Yu H. Sigma-1 receptor activity in primary sensory neurons is a critical driver of neuropathic pain. Gene Ther. 2022; 29 (1-2): 1-15.
48. Campbell J N, and Meyer R A. Mechanisms of neuropathic pain. Neuron. 2006; 52 (1): 77-92.
49. Schafers M, Lee D H, Brors D, Yaksh T L, and Sorkin L S. Increased sensitivity of injured and adjacent uninjured rat primary sensory neurons to exogenous tumor necrosis factor-alpha after spinal nerve ligation. J Neurosci. 2003; 23 (7): 3028-38.
50. Kim Y S, Anderson M, Park K, Zheng Q, Agarwal A, Gong C, et al. Coupled Activation of Primary Sensory Neurons Contributes to Chronic Pain. Neuron. 2016; 91 (5): 1085-96.
51. Shin S M, Itson-Zoske B, Cai Y, Qiu C, Pan B, Stucky C L, et al. Satellite glial cells in sensory ganglia express functional transient receptor potential ankyrin 1 that is sensitized in neuropathic and inflammatory pain. Mol Pain. 2020; 16: 1744806920925425.
52. Mogil J S. Sex differences in pain and pain inhibition: multiple explanations of a controversial phenomenon. Nat Rev Neurosci. 2012; 13 (12): 859-66.
53. In: Phillips J K, Ford M A, and Bonnie R J eds. Pain Management and the Opioid Epidemic: Balancing Societal and Individual Benefits and Risks of Prescription Opioid Use. Washington (DC); 2017.
54. Alles S R A, and Smith P A. Peripheral Voltage-Gated Cation Channels in Neuropathic Pain and Their Potential as Therapeutic Targets. Front Pain Res (Lausanne). 2021; 2:750583.
55. Kerckhove N, Pereira B, Soriot-Thomas S, Alchaar H, Deleens R, Hieng V S, et al. Efficacy and safety of a T-type calcium channel blocker in patients with neuropathic pain: A proof-of-concept, randomized, double-blind and controlled trial. Eur J Pain. 2018; 22 (7): 1321-30.
56. Raja S N, Ringkamp M, Guan Y, and Campbell J N. John J. Bonica Award Lecture: Peripheral neuronal hyperexcitability: the “low-hanging” target for safe therapeutic strategies in neuropathic pain. Pain. 2020; 161 Suppl 1: S14-S26.
57. Baron R. Mechanisms of disease: neuropathic pain—a clinical perspective. Nat Clin Pract Neurol. 2006; 2 (2): 95-106.
58. Theile J W, and Cummins T R. Recent developments regarding voltage-gated sodium channel blockers for the treatment of inherited and acquired neuropathic pain syndromes. Front Pharmacol. 2011; 2:54.
59. Ovsepian S V, and Waxman S G. Gene therapy for chronic pain: emerging opportunities in target-rich peripheral nociceptors. Nat Rev Neurosci. 2023; 24 (4): 252-65.
60. King A. A different path: resh strategies and targets for chronic pain could deliver much-needed replacements for opioid-based painkillers. Nature. 2019; 573 (Semptember 12): S4-S6.
61. Amir R, and Devor M. Chemically mediated cross-excitation in rat dorsal root ganglia. J Neurosci. 1996; 16 (15): 4733-41.
62. Cai W, Zhao Q, Shao J, Zhang J, Li L, Ren X, et al. MicroRNA-182 Alleviates Neuropathic Pain by Regulating Nav1.7 Following Spared Nerve Injury in Rats. Sci Rep. 2018; 8 (1): 16750.
63. Emery E C, Luiz A P, and Wood J N. Nav1.7 and other voltage-gated sodium channels as drug targets for pain relief. Expert Opin Ther Targets. 2016; 20 (8): 975-83.
64. Shields S D, Deng L, Reese R M, Dourado M, Tao J, Foreman O, et al. Insensitivity to Pain upon Adult-Onset Deletion of Nav1.7 or Its Blockade with Selective Inhibitors. J Neurosci. 2018; 38 (47): 10180-201.
65. Gold M S, and Gebhart G F. Nociceptor sensitization in pain pathogenesis. Nat Med. 2010; 16 (11): 1248-57.
66. Finnerup N B, Kuner R, and Jensen T S. Neuropathic Pain: From Mechanisms to Treatment. Physiol Rev. 2021; 101 (1): 259-301.
67. Pfirrmann C W, Oberholzer P A, Zanetti M, Boos N, Trudell D J, Resnick D, et al. Selective nerve root blocks for the treatment of sciatica: evaluation of injection site and effectiveness--a study with patients and cadavers. Radiology. 2001; 221 (3): 704-11.
68. Fischer G, Kostic S, Nakai H, Park F, Sapunar D, Yu H, et al. Direct injection into the dorsal root ganglion: technical, behavioral, and histological observations. J Neurosci Methods. 2011; 199 (1): 43-55.
69. Hoffmann T, Sharon O, Wittmann J, Carr R W, Vyshnevska A, Col R, et al. NaV1.7 and pain: contribution of peripheral nerves. Pain. 2018; 159 (3): 496-506.
70. Hordeaux J, Buza E L, Dyer C, Goode T, Mitchell T W, Richman L, et al. Adeno-Associated Virus-Induced Dorsal Root Ganglion Pathology. Hum Gene Ther. 2020; 31 (15-16): 808-18.
71. Beutler A S, and Reinhardt M. AAV for pain: steps towards clinical translation. Gene Ther. 2009; 16 (4): 461-9.
72. Pleticha J, Maus T P, and Beutler A S. Future Directions in Pain Management: Integrating Anatomically Selective Delivery Techniques With Novel Molecularly Selective Agents. Mayo Clin Proc. 2016; 91 (4): 522-33.
73. Pleticha J, Maus T P, Christner J A, Marsh M P, Lee K H, Hooten W M, et al. Minimally invasive convection-enhanced delivery of biologics into dorsal root ganglia: validation in the pig model and prospective modeling in humans. Technical note. J Neurosurg. 2014; 121 (4): 851-8.
74. Malfait A M, and Miller R J. Emerging Targets for the Management of Osteoarthritis Pain. Curr Osteoporos Rep. 2016; 14 (6): 260-8.
75. Alles S R A, Nascimento F, Lujan R, Luiz A P, Millet Q, Bangash M A, et al. Sensory neuron-derived NaV1.7 contributes to dorsal horn neuron excitability. Sci Adv. 2020; 6 (8): eaax4568.
76. Dong W, Zhang X, Liu W, Chen Y J, Huang J, Austin E, et al. A conserved polybasic domain mediates plasma membrane targeting of Lgl and its regulation by hypoxia. J Cell Biol. 2015; 211 (2): 273-86.
77. Kanwa N, De S K, Maity A, and Chakraborty A. Interaction of aliphatic amino acids with zwitterionic and charged lipid membranes: hydration and dehydration phenomena. Phys Chem Chem Phys. 2020; 22 (6): 3234-44.
78. Furukawa J, Fujitani N, Araki K, Takegawa Y, Kodama K, and Shinohara Y. A versatile method for analysis of serine/threonine posttranslational modifications by beta-elimination in the presence of pyrazolone analogues. Anal Chem. 2011; 83 (23): 9060-7.
79. Zhang Y, Qin Z, Sun W, Chu F, and Zhou F. Function of Protein S-Palmitoylation in Immunity and Immune-Related Diseases. Front Immunol. 2021; 12:661202.
80. Alioua A, Li M, Wu Y, Stefani E, and Toro L. Unconventional myristoylation of large-conductance Ca (2) (+)-activated K (+) channel (Slo1) via serine/threonine residues regulates channel surface expression. Proc Natl Acad Sci USA. 2011; 108 (26): 10744-9.
81. Beltran-Alvarez P, Espejo A, Schmauder R, Beltran C, Mrowka R, Linke T, et al. Protein arginine methyl transferases-3 and 5 increase cell surface expression of cardiac sodium channel. FEBS Lett. 2013; 587 (19): 3159-65.
82. Okada A K, Teranishi K, Ambroso M R, Isas J M, Vazquez-Sarandeses E, Lee J Y, et al. Lysine acetylation regulates the interaction between proteins and membranes. Nat Commun. 2021; 12 (1): 6466.
83. Tyagi S, Sarveswaran N, Higerd-Rusli G P, Liu S, Dib-Hajj F B, Waxman S G, et al. Conserved but not critical: Trafficking and function of Na (V) 1.7 are independent of highly conserved polybasic motifs. Front Mol Neurosci. 2023; 16: 1161028.
84. Gomez-Ospina N, Tsuruta F, Barreto-Chang O, Hu L, and Dolmetsch R. The C terminus of the L-type voltage-gated calcium channel Ca (V) 1.2 encodes a transcription factor. Cell. 2006; 127 (3): 591-606.
85. Lu L, Sirish P, Zhang Z, Woltz R L, Li N, Timofeyev V, et al. Regulation of gene transcription by voltage-gated L-type calcium channel, Cav1.3. J Biol Chem. 2015; 290 (8): 4663-76.
86. Ruhlmann A H, Korner J, Hausmann R, Bebrivenski N, Neuhof C, Detro-Dassen S, et al. Uncoupling sodium channel dimers restores the phenotype of a pain-linked Na (v) 1.7 channel mutation. Br J Pharmacol. 2020; 177 (19): 4481-96.
87. Clatot J, Hoshi M, Wan X, Liu H, Jain A, Shinlapawittayatorn K, et al. Voltage-gated sodium channels assemble and gate as dimers. Nat Commun. 2017; 8 (1): 2077.

We claim:

1. A polypeptide aptamer comprising a sequence selected from the group consisting of SEQ ID NOs: 1-9, 50, and 52, or a sequence with at least 85% identity to one of SEQ ID NOs: 1-9, 50, and 52.

2-4. (canceled)

5. The polypeptide aptamer of claim 1, wherein the polypeptide aptamer further comprises a detectable marker.

6.-7. (canceled)

8. The polypeptide aptamer of claim 5, wherein the polypeptide aptamer further comprises a linker.

9. The polypeptide aptamer of claim 8, wherein the linker comprises SEQ ID NO: 10.

10. The polypeptide aptamer of claim 9, wherein the detectable marker, linker, and sequence are a single continuous peptide sequence and are configured, from N-terminus to C-terminus: detectable marker, linker, sequence.

11. The polypeptide aptamer of claim 1, wherein the polypeptide aptamer binds to human Naᵥ1.7 channel protein.

12. A pharmaceutical composition comprising the polypeptide aptamer of claim 1 and a pharmaceutically acceptable carrier or excipient.

13. A polynucleotide comprising a nucleotide sequence encoding the polypeptide aptamer of claim 1.

14.-17. (canceled)

18. An infectious particle comprising the polynucleotide of claim 13.

19. The infectious particle of claim 18, wherein the infectious particle is a virus.

20. The infectious particle of claim 19, wherein the virus is an adeno-associated virus (AAV).

21. The infectious particle of claim 20, wherein in the adeno-associated virus (AAV) is an AAV type 6 (AAV6).

22. A pharmaceutical composition comprising the infectious particle of claim 18.

23. A method comprising contacting a cell with the polypeptide aptamer of claim 1.

24. A method of reducing or inhibiting stimulation of a neuron, the method comprising contacting a neuron with the polypeptide aptamer of claim 1.

25.-27. (canceled)

28. A method of treating neuropathic pain in a subject in need thereof, the method comprising administering a therapeutically effective amount of the pharmaceutical composition of claim 22 to the subject to treat neuropathic pain in the subject.

29. A method of treating pain due to a traumatic nerve injury in a subject in need thereof, the method comprising administering a therapeutically effective amount of the pharmaceutical composition of claim 22 to the subject to treat the pain due to the traumatic nerve injury in the subject.

30-36. (canceled)

37. A cell comprising a heterologous polynucleotide encoding a human Nav1.8 protein.

38.-45. (canceled)

46. A kit, system, or platform comprising the polypeptide aptamer of claim 1, or a polynucleotide encoding the polypeptide aptamer of claim 1, and reagents for performing electrophysiology experiments.

47. The kit, system, or platform of claim 46, wherein the electrophysiology experiments comprise patch clamp electrophysiology.

48. (canceled)


SEQUENCE LISTING (75 sequences)

SEQ ID NO: 1 - protein, 44 aa, synthetic construct
SSKSAKERRNRRKKKKQKMSSGEEKGDDEKLSKSGSEESIRKKS

SEQ ID NO: 2 - protein, 40 aa, synthetic construct
KRKQEEVSATIIQRAYRRYLRQHVKNISSIYIKDGDRDDD

SEQ ID NO: 3 - protein, 31 aa, synthetic construct
SEEKAKEHKDEKKDDEEEGPKPSSDLEAGKQ

SEQ ID NO: 4 - protein, 23 aa, synthetic construct
RGSLFSARRSSRTSLFSFKGRGR

SEQ ID NO: 5 - protein, 19 aa, synthetic construct
ESRRGSLFVPHRPRERRSS

SEQ ID NO: 6 - protein, 52 aa, synthetic construct
PGESDLEIMNTEELSSDSDSDYSKEKRNRSSSSECSTVDNPLPGEEEAEAEP

SEQ ID NO: 7 - protein, 37 aa, synthetic construct
KKLGGQDIFMTEEQKKYYNAMKKLGSKKPQKPIPRPG

SEQ ID NO: 8 - protein, 36 aa, synthetic construct
STISPPSYDSVTKPDQEKYETDKTEKEDKEKDESRK

SEQ ID NO: 9 - protein, 50 aa, synthetic construct
KSFSKKPKGSKDTKRTADPNNKEKNYISNRTLAEMSKDHNFLKEKDRISG

SEQ ID NO: 10 - protein, 21 aa, synthetic construct
GLRSRAQASNSAVDGTAGPGS

SEQ ID NO: 11 - DNA, 132 bp, synthetic construct
  source 1..132: mol_type = other DNA
agctcaaagagtgccaaggagagaagaaaccgaagaaagaaaaagaaacagaagatgtccagtggcgaggaaaagggtgacgatgagaagctgtccaagtcaggatcagaggaaagcatccgaaagaaaagt

SEQ ID NO: 12 - DNA, 126 bp, synthetic construct
  source 1..126: mol_type = other DNA
aagagaaaacaagaggaggtgtccgcgactatcattcagcgtgcttacagacggtatcgcctcagacaacacgtcaagaatatatcgagtatatacataaaagatggagacagggatgatgatttg

SEQ ID NO: 13 - DNA, 93 bp, synthetic construct
  source 1..93: mol_type = other DNA
tctgaagaaaaagccaaggaacacaaagacgaaaagaaagatgatgaggaagaaggccccaagcccagcagtgacttggaagctgggaaacag

SEQ ID NO: 14 - DNA, 69 bp, synthetic construct
  source 1..69: mol_type = other DNA
cgtgggtccctgttttctgccaggcgcagcagcaggacgagtctgttcagttttaaggggcgaggaaga

SEQ ID NO: 15 - DNA, 57 bp, synthetic construct
  source 1..57: mol_type = other DNA
gagagcagaaggggttcactattcgtaccccatagaccccgggagcggcgcagcagt

SEQ ID NO: 16 - DNA, 156 bp, synthetic construct
  source 1..156: mol_type = other DNA
cctggggagtctgatttggagattatgaacacagaagagcttagcagtgactcagacagtgactacagcaaagagaaacggaaccgatcaagctcttctgagtgcagcactgttgacaaccctctgccaggagaagaggaggctgaagcagagccc

SEQ ID NO: 17 - DNA, 114 bp, synthetic construct
  source 1..114: mol_type = other DNA
aaaaaaaagcttggaggtcaagatatctttatgacagaagaacagaagaaatactataatgcaatgaagaagcttgggtccaagaaaccacaaaaaccaattccaaggccaggg

SEQ ID NO: 18 - DNA, 108 bp, synthetic construct
  source 1..108: mol_type = other DNA
tcaaccatctcgccaccttcctatgacagtgtcacaaagccagatcaagagaaatatgaaacagacaaaacagagaaggaagacaaagagaaagatgaaagcaggaaa

SEQ ID NO: 19 - DNA, 162 bp, synthetic construct
  source 1..162: mol_type = other DNA
aaatcattttccaaaaagccaaagggctccaaggacacaaaacgaacagcagatcccaacaacaagaaagaaaactatatttcaaaccgtacccttgcggagatgagcaaggatcacaatttcctcaaagaaaaggataggatcagtggttatggcagcagt

SEQ ID NO: 20 - protein, 1955 aa, Homo sapiens
MEFPIGSLETNNFRRFTPESLVEIEKQIAAKQGTKKAREKHREQKDQEEKPRPQLDLKACNQLPKFYGELPAELIGEPLEDLDPFYSTHRTFMVLNKGRTISRFSATRALWLFSPFNLIRRTAIKVSVHSWFSLFITVTILVNCVCMTRTDLPEKIEYVFTVIYTFEALIKILARGFCLNEFTYLRDPWNWLDFSVITLAYVGTAIDLRGISGLRTFRVLRALKTVSVIPGLKVIVGALIHSVKKLADVTILTIFCLSVFALVGLQLFKGNLKNKCVKNDMAVNETTNYSSHRKPDIYINKRGTSDPLLCGNGSDSGHCPDGYICLKTSDNPDFNYTSFDSFAWAFLSLFRLMTQDSWERLYQQTLRTSGKIYMIFFVLVIFLGSFYLVNLILAVVTMAYEEQNQATTDEIEAKEKKFQEALEMLRKEQEVLAALGIDTTSLHSHNGSPLTSKNASERRHRIKPRVSEGSTEDNKSPRSDPYNQRRMSFLGLASGKRRASHGSVFHFRSPGRDISLPEGVTDDGVFPGDHESHRGSLLLGGGAGQQGPLPRSPLPQPSNPDSRHGEDEHQPPPTSELAPGAVDVSAFDAGQKKTFLSAEYLDEPFRAQRAMSVVSIITSVLEELEESEQKCPPCLTSLSQKYLIWDCCPMWVKLKTILFGLVTDPFAELTITLCIVVNTIFMAMEHHGMSPTFEAMLQIGNIVFTIFFTAEMVFKIIAFDPYYYFQKKWNIFDCIIVTVSLLELGVAKKGSLSVLRSFRLLRVFKLAKSWPTLNTLIKIIGNSVGALGNLTIILAIIVFVFALVGKQLLGENYRNNRKNISAPHEDWPRWHMHDFFHSFLIVFRILCGEWIENMWACMEVGQKSICLILFLTVMVLGNLVVLNLFIALLLNSFSADNLTAPEDDGEVNNLQVALARIQVFGHRTKQALCSFFSRSCPFPQPKAEPELVVKLPLSSSKAENHIAANTARGSSGGLQAPRGPRDEHSDFIANPTVWVSVPIAEGESDLDDLEDDGGEDAQSFQQEVIPKGQEQLQQVERCGDHLTPRSPGTGTSSEDLAPSLGETWKDESVPQVPAEGVDDTSSSEGSTVDCLDPEEILRKIPELADDLEEPDDCFTEGCIRHCPCCKLDTTKSPWDVGWQVRKTCYRIVEHSWFESFIIFMILLSSGSLAFEDYYLDQKPTVKALLEYTDRVFTFIFVFEMLLKWVAYGFKKYFTNAWCWLDFLIVNISLISLTAKILEYSEVAPIKALRTLRALRPLRALSRFEGMRVVVDALVGAIPSIMNVLLVCLIFWLIFSIMGVNLFAGKFWRCINYTDGEFSLVPLSIVNNKSDCKIQNSTGSFFWVNVKVNFDNVAMGYLALLQVATFKGWMDIMYAAVDSREVNMQPKWEDNVYMYLYFVIFIIFGGFFTLNLFVGVIIDNFNQQKKKLGGQDIFMTEEQKKYYNAMKKLGSKKPQKPIPRPLNKFQGFVFDIVTRQAFDITIMVLICLNMITMMVETDDQSEEKTKILGKINQFFVAVFTGECVMKMFALRQYYFTNGWNVFDFIVVVLSIASLIFSAILKSLQSYFSPTLFRVIRLARIGRILRLIRAAKGIRTLLFALMMSLPALFNIGLLLFLVMFIYSIFGMSSFPHVRWEAGIDDMFNFQTFANSMLCLFQITTSAGWDGLLSPILNTGPPYCDPNLPNSNGTRGDCGSPAVGIIFFTTYIIISFLIVVNMYIAVILENFNVATEESTEPLSEDDFDMFYETWEKFDPEATQFITFSALSDFADTLSGPLRIPKPNRNILIQMDLPLVPGDKIHCLDILFAFTKNVLGESGELDSLKANMEEKFMATNLSKSSYEPIATTLRWKQEDISATVIQKAYRSYVLHRSMALSNTPCVPRAEEEAASLPDEGFVAFTANENCVLPDKSETASATSFPPSYESVTRGLSDRVNMRTSSSIQNEDEATSMELIAPGP

SEQ ID NO: 21 - DNA, 5871 bp, Homo sapiens
  source 1..5871: mol_type = genomic DNA
atggaattccccattggatccctcgaaactaacaacttccgtcgctttactccggagtcactggtggagatagagaagcaaattgctgccaagcagggaacaaagaaagccagagagaagcatagggagcagaaggaccaagaagagaagcctcggccccagctggacttgaaagcctgcaaccagctgcccaagttctatggtgagctcccagcagaactgatcggggagcccctggaggatctagatccgttctacagcacacaccggacatttatggtgctgaacaaagggaggaccatttcccggtttagtgccactcgggccctgtggctattcagtcctttcaacctgatcagaagaacggccatcaaagtgtctgtccactcgtggttcagtttatttattacggtcactattttggttaattgtgtgtgcatgacccgaactgaccttccagagaaaattgaatatgtcttcactgtcatttacacctttgaagccttgataaagatactggcaagaggattttgtctaaatgagttcacgtacctgagagatccttggaactggctggattttagcgtcattaccctggcatatgttggcacagcaatagatctccgtgggatctcaggcctgcggacattcagagttcttagagcattaaaaacagtttctgtgatcccaggcctgaaggtcattgtgggggccctgattcactcagtgaagaaactggctgatgtgaccatcctcaccatcttctgcctaagtgtttttgccttggtggggctgcaactcttcaagggcaacctcaaaaataaatgtgtcaagaatgacatggctgtcaatgagacaaccaactactcatctcacagaaaaccagatatctacataaataagcgaggcacttctgaccccttactgtgtggcaatggatctgactcaggccactgccctgatggttatatctgccttaaaacttctgacaacccggattttaactacaccagctttgattcctttgcttgggctttcctctcactgttccgcctcatgacacaggattcctgggaacgcctctaccagcagaccctgaggacttctgggaaaatctatatgatcttttttgtgctcgtaatcttcctgggatctttctacctggtcaacttgatcttggctgtagtcaccatggcgtatgaggagcagaaccaggcaaccactgatgaaattgaagcaaaggagaagaagttccaggaggccctcgagatgctccggaaggagcaggaggtgctagcagcactagggattgacacaacctctctccactcccacaatggatcacctttaacctccaaaaatgccagtgagagaaggcatagaataaagccaagagtgtcagagggctccacagaagacaacaaatcaccccgctctgatccttacaaccagcgcaggatgtcttttctaggcctcgcctctggaaaacgccgggctagtcatggcagtgtgttccatttccggtcccctggccgagatatctcactccctgagggagtcacagatgatggagtctttcctggagaccacgaaagccatcggggctctctgctgctgggtgggggtgctggccagcaaggccccctccctagaagccctcttcctcaacccagcaaccctgactccaggcatggagaagatgaacaccaaccgccgcccactagtgagcttgcccctggagctgtcgatgtctcggcattcgatgcaggacaaaagaagactttcttgtcagcagaatacttagatgaacctttccgggcccaaagggcaatgagtgttgtcagtatcataacctccgtccttgaggaactcgaggagtctgaacagaagtgcccaccctgcttgaccagcttgtctcagaagtatctgatctgggattgctgccccatgtgggtgaagctcaagacaattctctttgggcttgtgacggatccctttgcagagctcaccatcaccttgtgcatcgtggtgaacaccatcttcatggccatggagcaccatggcatgagccctaccttcgaagccatgctccagataggcaacatcgtctttaccatattttttactgctgaaatggtcttcaaaatcattgccttcgacccatactattatttccagaagaagtggaatatctttgactgcatcatcgtcactgtgagtctgctagagctgggcgtggccaagaagggaagcctgtctgtgctgcggagcttccgcttgctgcgcgtattcaagctggccaaatcctggcccaccttaaacacactcatcaagatcatcggaaactcagtgggggcactggggaacctcaccatcatcctggccatcattgtctttgtctttgctctggttggcaagcagctcctaggggaaaactaccgtaacaaccgaaaaaatatctccgcgccccatgaagactggccccgctggcacatgcacgacttcttccactctttcctcattgtcttccgtatcctctgtggagagtggattgagaacatgtgggcctgcatggaagttggccaaaaatccatatgcctcatccttttcttgacggtgatggtgctagggaacctggtggtgcttaacctgttcatcgccctgctattgaactctttcagtgctgacaacctcacagccccggaggacgatggggaggtgaacaacctgcaggtggccctggcacggatccaggtctttggccatcgtaccaaacaggctctttgcagcttcttcagcaggtcctgcccattcccccagcccaaggcagagcctgagctggtggtgaaactcccactctccagctccaaggctgagaaccacattgctgccaacactgccagggggagctctggagggctccaagctcccagaggccccagggatgagcacagtgacttcatcgctaatccgactgtgtgggtctctgtgcccattgctgagggtgaatctgatcttgatgacttggaggatgatggtggggaagatgctcagagcttccagcaggaagtgatccccaaaggacaggagcagctgcagcaagtcgagaggtgtggggaccacctgacacccaggagcccaggcactggaacatcttctgaggacctggctccatccctgggtgagacgtggaaagatgagtctgttcctcaggtccctgctgagggagtggacgacacaagctcctctgagggcagcacggtggactgcctagatcctgaggaaatcctgaggaagatccctgagctggcagatgacctggaagaaccagatgactgcttcacagaaggatgcattcgccactgtccctgctgcaaactggataccaccaagagtccatgggatgtgggctggcaggtgcgcaagacttgctaccgtatcgtggagcacagctggtttgagagcttcatcatcttcatgatcctgctcagcagtggatctctggcctttgaagactattacctggaccagaagcccacggtgaaagctttgctggagtacactgacagggtcttcacctttatctttgtgttcgagatgctgcttaagtgggtggcctatggcttcaaaaagtacttcaccaatgcctggtgctggctggacttcctcattgtgaatatctcactgataagtctcacagcgaagattctggaatattctgaagtggctcccatcaaagcccttcgaacccttcgcgctctgcggccactgcgggctctttctcgatttgaaggcatgcgggtggtggtggatgccctggtgggcgccatcccatccatcatgaatgtcctcctcgtctgcctcatcttctggctcatcttcagcatcatgggtgtgaacctcttcgcagggaagttttggaggtgcatcaactataccgatggagagttttcccttgtacctttgtcgattgtgaataacaagtctgactgcaagattcaaaactccactggcagcttcttctgggtcaatgtgaaagtcaactttgataatgttgcaatgggttaccttgcacttctgcaggtggcaacctttaaaggctggatggacattatgtatgcagctgttgattcccgggaggtcaacatgcaacccaagtgggaggacaacgtgtacatgtatttgtactttgtcatcttcatcatttttggaggcttcttcacactgaatctctttgttggggtcataattgacaacttcaatcaacagaaaaaaaagttagggggccaggacatcttcatgacagaggagcagaagaaatactacaatgccatgaagaagttgggctccaagaagccccagaagcccatcccacggcccctgaacaagttccagggttttgtctttgacatcgtgaccagacaagcttttgacatcaccatcatggtcctcatctgcctcaacatgatcaccatgatggtggagactgatgaccaaagtgaagaaaagacgaaaattctgggcaaaatcaaccagttctttgtggccgtcttcacaggcgaatgtgtcatgaagatgttcgctttgaggcagtactacttcacaaatggctggaatgtgtttgacttcattgtggtggttctctccattgcgagcctgattttttctgcaattcttaagtcacttcaaagttacttctccccaacgctcttcagagtcatccgcctggcccgaattggccgcatcctcagactgatccgagcggccaaggggatccgcacactgctctttgccctcatgatgtccctgcctgccctcttcaacatcgggctgttgctattccttgtcatgttcatctactctatcttcggtatgtccagctttccccatgtgaggtgggaggctggcatcgacgacatgttcaacttccagaccttcgccaacagcatgctgtgcctcttccagattaccacgtcggccggctgggatggcctcctcagccccatcctcaacacagggcccccctactgtgaccccaatctgcccaacagcaatggcaccagaggggactgtgggagcccagccgtaggcatcatcttcttcaccacctacatcatcatctccttcctcatcgtggtcaacatgtacattgcagtgattctggagaacttcaatgtggccacggaggagagcactgagcccctgagtgaggacgactttgacatgttctatgagacctgggagaagtttgacccagaggccactcagtttattaccttttctgctctctcggactttgcagacactctctctggtcccctgagaatcccaaaacccaatcgaaatatactgatccagatggacctgcctttggtccctggagataagatccactgcttggacatcctttttgctttcaccaagaatgtcctaggagaatccggggagttggattctctgaaggcaaatatggaggagaagtttatggcaactaatctttcaaaatcatcctatgaaccaatagcaaccactctccgatggaagcaagaagacatttcagccactgtcattcaaaaggcctatcggagctatgtgctgcaccgctccatggcactctctaacaccccatgtgtgcccagagctgaggaggaggctgcatcactcccagatgaaggttttgttgcattcacagcaaatgaaaattgtgtactcccagacaaatctgaaactgcttctgccacatcattcccaccgtcctatgagagtgtcactagaggccttagtgatagagtcaacatgaggacatctagctcaatacaaaatgaagatgaagccaccagtatggagctgattgcccctgggccctagtga

SEQ ID NO: 22 - DNA, 11985 bp, synthetic construct
  source 1..11985: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgccaccatggaattccccattggatccctcgaaactaacaacttccgtcgctttactccggagtcactggtggagatagagaagcaaattgctgccaagcagggaacaaagaaagccagagagaagcatagggagcagaaggaccaagaagagaagcctcggccccagctggacttgaaagcctgcaaccagctgcccaagttctatggtgagctcccagcagaactgatcggggagcccctggaggatctagatccgttctacagcacacaccggacatttatggtgctgaacaaagggaggaccatttcccggtttagtgccactcgggccctgtggctattcagtcctttcaacctgatcagaagaacggccatcaaagtgtctgtccactcgtggttcagtttatttattacggtcactattttggttaattgtgtgtgcatgacccgaactgaccttccagagaaaattgaatatgtcttcactgtcatttacacctttgaagccttgataaagatactggcaagaggattttgtctaaatgagttcacgtacctgagagatccttggaactggctggattttagcgtcattaccctggcatatgttggcacagcaatagatctccgtgggatctcaggcctgcggacattcagagttcttagagcattaaaaacagtttctgtgatcccaggcctgaaggtcattgtgggggccctgattcactcagtgaagaaactggctgatgtgaccatcctcaccatcttctgcctaagtgtttttgccttggtggggctgcaactcttcaagggcaacctcaaaaataaatgtgtcaagaatgacatggctgtcaatgagacaaccaactactcatctcacagaaaaccagatatctacataaataagcgaggcacttctgaccccttactgtgtggcaatggatctgactcaggccactgccctgatggttatatctgccttaaaacttctgacaacccggattttaactacaccagctttgattcctttgcttgggctttcctctcactgttccgcctcatgacacaggattcctgggaacgcctctaccagcagaccctgaggacttctgggaaaatctatatgatcttttttgtgctcgtaatcttcctgggatctttctacctggtcaacttgatcttggctgtagtcaccatggcgtatgaggagcagaaccaggcaaccactgatgaaattgaagcaaaggagaagaagttccaggaggccctcgagatgctccggaaggagcaggaggtgctagcagcactagggattgacacaacctctctccactcccacaatggatcacctttaacctccaaaaatgccagtgagagaaggcatagaataaagccaagagtgtcagagggctccacagaagacaacaaatcaccccgctctgatccttacaaccagcgcaggatgtcttttctaggcctcgcctctggaaaacgccgggctagtcatggcagtgtgttccatttccggtcccctggccgagatatctcactccctgagggagtcacagatgatggagtctttcctggagaccacgaaagccatcggggctctctgctgctgggtgggggtgctggccagcaaggccccctccctagaagccctcttcctcaacccagcaaccctgactccaggcatggagaagatgaacaccaaccgccgcccactagtgagcttgcccctggagctgtcgatgtctcggcattcgatgcaggacaaaagaagactttcttgtcagcagaatacttagatgaacctttccgggcccaaagggcaatgagtgttgtcagtatcataacctccgtccttgaggaactcgaggagtctgaacagaagtgcccaccctgcttgaccagcttgtctcagaagtatctgatctgggattgctgccccatgtgggtgaagctcaagacaattctctttgggcttgtgacggatccctttgcagagctcaccatcaccttgtgcatcgtggtgaacaccatcttcatggccatggagcaccatggcatgagccctaccttcgaagccatgctccagataggcaacatcgtctttaccatattttttactgctgaaatggtcttcaaaatcattgccttcgacccatactattatttccagaagaagtggaatatctttgactgcatcatcgtcactgtgagtctgctagagctgggcgtggccaagaagggaagcctgtctgtgctgcggagcttccgcttgctgcgcgtattcaagctggccaaatcctggcccaccttaaacacactcatcaagatcatcggaaactcagtgggggcactggggaacctcaccatcatcctggccatcattgtctttgtctttgctctggttggcaagcagctcctaggggaaaactaccgtaacaaccgaaaaaatatctccgcgccccatgaagactggccccgctggcacatgcacgacttcttccactctttcctcattgtcttccgtatcctctgtggagagtggattgagaacatgtgggcctgcatggaagttggccaaaaatccatatgcctcatccttttcttgacggtgatggtgctagggaacctggtggtgcttaacctgttcatcgccctgctattgaactctttcagtgctgacaacctcacagccccggaggacgatggggaggtgaacaacctgcaggtggccctggcacggatccaggtctttggccatcgtaccaaacaggctctttgcagcttcttcagcaggtcctgcccattcccccagcccaaggcagagcctgagctggtggtgaaactcccactctccagctccaaggctgagaaccacattgctgccaacactgccagggggagctctggagggctccaagctcccagaggccccagggatgagcacagtgacttcatcgctaatccgactgtgtgggtctctgtgcccattgctgagggtgaatctgatcttgatgacttggaggatgatggtggggaagatgctcagagcttccagcaggaagtgatccccaaaggacagcaggagcagctgcagcaagtcgagaggtgtggggaccacctgacacccaggagcccaggcactggaacatcttctgaggacctggctccatccctgggtgagacgtggaaagatgagtctgttcctcaggtccctgctgagggagtggacgacacaagctcctctgagggcagcacggtggactgcctagatcctgaggaaatcctgaggaagatccctgagctggcagatgacctggaagaaccagatgactgcttcacagaaggatgcattcgccactgtccctgctgcaaactggataccaccaagagtccatgggatgtgggctggcaggtgcgcaagacttgctaccgtatcgtggagcacagctggtttgagagcttcatcatcttcatgatcctgctcagcagtggatctctggcctttgaagactattacctggaccagaagcccacggtgaaagctttgctggagtacactgacagggtcttcacctttatctttgtgttcgagatgctgcttaagtgggtggcctatggcttcaaaaagtacttcaccaatgcctggtgctggctggacttcctcattgtgaatatctcactgataagtctcacagcgaagattctggaatattctgaagtggctcccatcaaagcccttcgaacccttcgcgctctgcggccactgcgggctctttctcgatttgaaggcatgcgggtggtggtggatgccctggtgggcgccatcccatccatcatgaatgtcctcctcgtctgcctcatcttctggctcatcttcagcatcatgggtgtgaacctcttcgcagggaagttttggaggtgcatcaactataccgatggagagttttcccttgtacctttgtcgattgtgaataacaagtctgactgcaagattcaaaactccactggcagcttcttctgggtcaatgtgaaagtcaactttgataatgttgcaatgggttaccttgcacttctgcaggtggcaacctttaaaggctggatggacattatgtatgcagctgttgattcccgggaggtcaacatgcaacccaagtgggaggacaacgtgtacatgtatttgtactttgtcatcttcatcatttttggaggcttcttcacactgaatctctttgttggggtcataattgacaacttcaatcaacagaaaaaaaagttagggggccaggacatcttcatgacagaggagcagaagaaatactacaatgccatgaagaagttgggctccaagaagccccagaagcccatcccacggcccctgaacaagttccagggttttgtctttgacatcgtgaccagacaagcttttgacatcaccatcatggtcctcatctgcctcaacatgatcaccatgatggtggagactgatgaccaaagtgaagaaaagacgaaaattctgggcaaaatcaaccagttctttgtggccgtcttcacaggcgaatgtgtcatgaagatgttcgctttgaggcagtactacttcacaaatggctggaatgtgtttgacttcattgtggtggttctctccattgcgagcctgattttttctgcaattcttaagtcacttcaaagttacttctccccaacgctcttcagagtcatccgcctggcccgaattggccgcatcctcagactgatccgagcggccaaggggatccgcacactgctctttgccctcatgatgtccctgcctgccctcttcaacatcgggctgttgctattccttgtcatgttcatctactctatcttcggtatgtccagctttccccatgtgaggtgggaggctggcatcgacgacatgttcaacttccagaccttcgccaacagcatgctgtgcctcttccagattaccacgtcggccggctgggatggcctcctcagccccatcctcaacacagggcccccctactgtgaccccaatctgcccaacagcaatggcaccagaggggactgtgggagcccagccgtaggcatcatcttcttcaccacctacatcatcatctccttcctcatcgtggtcaacatgtacattgcagtgattctggagaacttcaatgtggccacggaggagagcactgagcccctgagtgaggacgactttgacatgttctatgagacctgggagaagtttgacccagaggccactcagtttattaccttttctgctctctcggactttgcagacactctctctggtcccctgagaatcccaaaacccaatcgaaatatactgatccagatggacctgcctttggtccctggagataagatccactgcttggacatcctttttgctttcaccaagaatgtcctaggagaatccggggagttggattctctgaaggcaaatatggaggagaagtttatggcaactaatctttcaaaatcatcctatgaaccaatagcaaccactctccgatggaagcaagaagacatttcagccactgtcattcaaaaggcctatcggagctatgtgctgcaccgctccatggcactctctaacaccccatgtgtgcccagagctgaggaggaggctgcatcactcccagatgaaggttttgttgcattcacagcaaatgaaaattgtgtactcccagacaaatctgaaactgcttctgccacatcattcccaccgtcctatgagagtgtcactagaggccttagtgatagagtcaacatgaggacatctagctcaatacaaaatgaagatgaagccaccagtatggagctgattgcccctgggcccagaaaacggaggggatccggaagcggagctactaacttcagcctgctgaagcaggctggagacgtggaggagaaccctggacctgaattccacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggcgccaagtaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 23 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 24 - protein, 215 aa, Homo sapiens
MHRDAWLPRPAFSLTGLSLFFSLVPPGRSMEVTVPATLNVLNGSDARLPCTFNSCYTVNHKQFSLNWTYQECNNCSEEMFLQFRMKIINLKLERFQDRVEFSGNPSKYDVSVMLRNVQPEDEGIYNCYIMNPPDRHRGHGKIHLQVLMEEPPERDSTVAVIVGASVGGFLAVVILVLMVVKCVRRKKEQKLSTDDLKTEEEGKTDGEGNPDDGAK

SEQ ID NO: 25 - DNA, 4937 bp, Homo sapiens
  source 1..4937: mol_type = genomic DNA
acacactccctctctctctctttttagcagcaacatacaagccggccatattagagagatggaaataaagcttccttaatgttgtatatgtctttgaagtacatccgtgcatttttttttagcatccaaccattcctcccttgtagttctcgccccctcaaatcaccctctcccgtagcccacccgactaacatctcagtctctgaaaatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggcgccaagtagtgggtggccggccctgcagcctcccgtgtcccgtctcctcccctctccgccctgtacagtgaccctgcctgctcgctcttggtgtgcttcccgtgacctaggaccccagggcccacctggggcctcctgaacccccgacttcgtatctcccaccctgcaccaagagtgacccactctcttccatccgagaaacctgccatgctctgggacgtgtgggccctggggagaggagagaaagggctcccacctgccagtccctggggggaggcaggaggcacatgtgagggtccccagagagaagggagtgggtgggcaggggtagaggagggggccgctgtccacctgcccagtgcttggcctggcagtggcttcagagaggacctggtggggagggagggctttcctgtgctgacagcgctccctcaggagggccttggcctggcacggctgtgctcctcccctgctcccagcccagagcagccatcaggctggaggtgacgatgagttcctgaaacttggaggggcatgttaaagggatgactgtgcattccagggcactgacggaaagccagggctgcaggcaaagctggacatgtgccctggcccaggaggccatgttgggccctcgtttccattgctagtggcctccttggggctcccgttggctcctaatcccttaggactgtggatgaggccagactggaagagcagctccaggtagggggccatgtttcccagcggggacccaccaacagaggccagtttcaaagtcagctgaggggctgaggggtggggctccatggtgaatgcaggttgctgcaggctctgccttctccatggggtaaccaccctcgcctgggcaggggcagccaaggctgggaaatgaggaggccatgcacagggtggggcagctttctttggggcttcagtgagaactctcccagttgcccttggtggggtttccacctggcttttggctacagagagggaagggaaagcctgaggccggcataaggggaggccttggaacctgagctgccaatgccagccctgtcccatctgcagccacactactcgctcctctcccaacaactcccttcgtggggacaaaagtgacaattgtaggccaggcacagtggctcacgcctgtaatcccagcactttgggaggccaaggcgggtggattacctccatctgtttagtagaaatgggcaaaaccccatctctactaaaaatacaagaattagctgggcgtggtggcgtgtgcctgtaatcccagctatttgggaggctgaggcaggagaatcgcttgagcccgggaagcagaggttgcagtgaactgagatagtgatagtgccactgcaattcagcctgggtgacatagagagactccatctcaaaaaaaaaaaaaaaaaaaaaaaaaggcaattgtgcggagccaaccatcactgccattacactcacattcaatggaaattgattaggcctgagagaaggagcaagaggcagaaacactgactctgagaccatgaccttggaagatcctcagagatcaactagccaagtcactcacttaaaatggggaaactgaggttccaactgacagaaactgagggtcctgccctcttctctggctcctcagtctccagtttgaagcaacagagccatcattttcccagtgggaggggcctctgtgtcctggtctatgatggggcgagttggaggggtgggcttctctagccctgccccctcccaggagcccagcccctctggccaggactgaggaggagtggagggaagcagggagggagggagcaagtgtgaagggcacagctgctggcaaatgccccattccctgccccagcacttcccagcctttccaggagctctgccccacccctactcaaggagtcaggtgcccagggtagggaataagtctaggaaagggggtggagacaggaccgtgacagtctcagggaccaatccctgcctctgcccatcagattgccaatttggggagacagcaatatcatctgggagattcagcttaatttgcttcaattcagcaaccatttattgagcatctattatgtgccaggcactgtgccgggcactggggatatacacaaagccataagcactggggactgccaggttggcactgtagggaccaagattaactttttggtgcatctaattccacctctgcaagacactgcttccaaggctacagttggggaagatgaagagattctagaatctgtgccctgggaagggagaaacagcagggaagaggggcaatacaacagggtcaagggtgcagaggccaggcagagtgggtgcagagtgggggatgggcaaccagcctggtaacacctgggtgaggtgagcgagtcaagcccatctggaaatgctaaactcatcagctcagtacctaactgccaatcaacacactgccaatgcgggtgggctaagccgccaggatgcctgcagcagatggacgctgcccatccttgagccagccagctgggggcagagaggtgggtggcgggcacctcatgccagctgagaccaagagtggttgtatacatttctgcagcttgcagatttcatttttttggtcaccctttaagtgtccttctctcccaggatgagtgaggagctgtgctgatgttgggaacttatctttttccaggatccataacatttaggggaattatttgcgggctttcctagtgtattctaagaagttctaactctccatcaagaagcccaagattcctggagtctttttttcctctgccctgatcaactggggcccaggaaaagcaatgtctcttcacgggcctcagggccccagcagatggggctgggtcaggaaccatctctgtcccaggtgctcagccctcatatgcattttcttctggagcctcttagaatctcctccagaggactcatagagctgcctcccccaggatggtgaagttgggagcaaaatggggcttctgtaagtagaggtggaaaaccagagagatgtttccacagcagccatttccctaccctgtttcctaacttatgctgacaagttcttcctcttcctaccgtggggttcgagccccacttctggttgcatgttcccaccaccacgctctgctggttttagtcggctgactttctcaactgccccagcctctcctgctctctcagaacctccctctgcccccaccccccatctgtctcttcctcctaattaagggccactgcaaagagagacgggctggctaacccatgggatttagcttataagaacaaagcacaattattcaggactgaactgctttgaggtgccttggaactggctttggaaatagctatctgccccctagctatggactcttcacagttatttccagtttataaaaaggaaaagctgatcggatccatgtttctctctgcgagtctccttggggaggttgggctggtaaatatttgcaaggtcaggttaagcagacagacaattgaaaatactgggcaggtccaaaaggatagttccaggccataggaatggcttggtgcagttaacgggtgatgaagcaagaaatcttccttcctacttatgtcttaaagatggacagcctgtgtctcttgctgtgcctcttgctggcgtcttcctttattgaagcttgctgatgcactgtacatagtctctctctctaggtatagatatattatatatacaaatatcaaacatctcaccacacccaccccaaaagttacactgtgtgtgtgtggggaggtgtaataacttctcagtgttttgagttgattcaccaaaggcaaattttggaatgttcttgcgctttgtgcacttggaggggaaaacccaactacatatgcactttcttggttgtgtgtgctatagagaggttggaaggggtggttggaaggggagagaaactgagatggccctctgtgactcgactgctgggatgtatctgcttttgggagcagactgagtttcttttgcaatttgtcttattgtttgggtctactggcaacaataaactatctcccctgaaaa

SEQ ID NO: 26 - DNA, 84 bp, synthetic construct
  source 1..84: mol_type = other DNA
agaaaacggaggggatccggaagcggagctactaacttcagcctgctgaagcaggctggagacgtggaggagaaccctggacct

SEQ ID NO: 27 - protein, 7 aa, Synthetic construct
PRRARSV

SEQ ID NO: 28 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 29 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - protein, 44 aa, synthetic construct; identical to SEQ ID NO: 1

SEQ ID NO: 31 - protein, 44 aa, synthetic construct
SSKSAKERRNRRKKKKQKLSSGEEKGDDEKLSKSGSEESIRKKS

SEQ ID NO: 32 - protein, 45 aa, synthetic construct
SSKSAKERRNRRKKKNQKKLSSGEEKGDAEKLSKSESEDSIRRKS

SEQ ID NO: 33 - protein, 46 aa, synthetic construct
SSKSAKERRNRRKKRKQKELSEGEEKGDPEKVFKSESEDGGMRRKA

SEQ ID NO: 34 - protein, 45 aa, synthetic construct
SSKSAKEWRNRRKKRRQREHLEGNHRADGDRFPKSESEDSVKRRS

SEQ ID NO: 35 - protein, 44 aa, synthetic construct
SSKSAKERRNRRKKRKQKEQSGGEEKDDDEFHKSESEDSIRRKG

SEQ ID NO: 36 - protein, 71 aa, synthetic construct
SSFSPKKRKFFGSKTRKSFFMRGSKTAQASASDSEDDASKNPQLLEQTKRLSQNLPVDLFDEHVDPLHRQR

SEQ ID NO: 37 - protein, 53 aa, synthetic construct
SPLASKNANERRPRVKSRVSEGSTDDNRSPQSDPYNQRRMSFLGLSSGRRRAS

SEQ ID NO: 38 - protein, 61 aa, synthetic construct
SPLAPVTNHERKSKRRKRLSSGTEDGGDDRLPKSDSEDGPRALNQLSLTHGLSRTSMRPRS

SEQ ID NO: 39 - protein, 21 aa, synthetic construct
TDATSSTTSPPSYDSVLKPDK

SEQ ID NO: 40 - protein, 22 aa, synthetic construct
DIGSETEFADDEHSIFGDNESR

SEQ ID NO: 41 - protein, 15 aa, synthetic construct
ASILTNTVEELEESR

SEQ ID NO: 42 - protein, 11 aa, synthetic construct
VSYEPITTTLK

SEQ ID NO: 43 - protein, 10 aa, synthetic construct
ELEFQQMLDR

SEQ ID NO: 44 - protein, 11 aa, synthetic construct
TIVGALIQSVK

SEQ ID NO: 45 - protein, 9 aa, synthetic construct
INDDCTLPR

SEQ ID NO: 46 - protein, 9 aa, synthetic construct
YFYYLEGSK

SEQ ID NO: 47 - protein, 44 aa, synthetic construct
AAKAAKERRNRRKKKKQKMAAGEEKGDDEKLAKAGAEEAIRKKA

SEQ ID NO: 48 - protein, 44 aa, synthetic construct
SSKSAKEAANAAAAAAQAMSSGEEKGDDEKLSKSGSEESIRKKS

SEQ ID NO: 49 - protein, 44 aa, synthetic construct
AAKAAKERRNRRKKKKQKMSSGEEKGDDEKLSKSGSEESIRKKS

SEQ ID NO: 50 - protein, 44 aa, synthetic construct
SSKSAKERRNRRKKKKQKMAAGEEKGDDEKLSKSGSEESIRKKS

SEQ ID NO: 51 - protein, 44 aa, synthetic construct
SSKSAKERRNRRKKKKQKMSSGEEKGDDEKLAKAGAEESIRKKS

SEQ ID NO: 52 - protein, 44 aa, synthetic construct
SSKSAKERRNRRKKKKQKMSSGEEKGDDEKLSKSGSEEAIRKKA

SEQ ID NO: 53 - protein, 38 aa, synthetic construct
PGESDLEIMNTEELSSDSDSDYSKEKRNRSSSSECSTV

SEQ ID NO: 54 - protein, 39 aa, synthetic construct
PGESDLEIMNTEELSSKSKSKYSKEKRNRSSSSECSTVD

SEQ ID NO: 55 - protein, 38 aa, synthetic construct
PGESDLENMNTEELSSDSDSDYSKERRNRSSSSECSTV

SEQ ID NO: 56 - protein, 38 aa, synthetic construct
PGESDLENMNAEELSSDSDSEYSKVRLNRSSSSECSTV

SEQ ID NO: 57 - protein, 39 aa, synthetic construct
PGESDLEIMNTEELSSDSDSDYSKEKRNRSSSSECSTVD

SEQ ID NO: 58 - protein, 39 aa, synthetic construct
VGESDFENLNTEDVSSESDPEGSKDKLDDTSSSEGSTID

SEQ ID NO: 59 - protein, 39 aa, synthetic construct
VGESDFENLNTEEFSSESELEESKEKLNATSSSEGSTVD

SEQ ID NO: 60 - protein, 39 aa, synthetic construct
LGESDFENLTNEEFSSESDMEESKEKLNATSSSEGSTVD

SEQ ID NO: 61 - protein, 40 aa, synthetic construct
VGESDFENLNTEDFSSESDLEESKEKLNESSSSSEGSTVD

SEQ ID NO: 62 - protein, 25 aa, synthetic construct
PGESDLEIMNTEELSSDSDSDYSKE

SEQ ID NO: 63 - protein, 45 aa, synthetic construct
EGESDLDELEEDMEQASQSSWQEEDPKGQQEQLPQVQKCENHQAA

SEQ ID NO: 64 - protein, 42 aa, synthetic construct
EVEDDVEYCGEGGALPTSQHSAGVQAGDLPPETKQLTSPDDQ

SEQ ID NO: 65 - protein, 12 aa, synthetic construct
KNRSSSSECSTV

SEQ ID NO: 66 - protein, 45 aa, synthetic construct
RSPASMMSSEDLAPYLGESWKRKDSPQVPAEGVDDTSSSEGSTVD

SEQ ID NO: 67 - protein, 36 aa, synthetic construct
GVEMEVFSEEDLHLSIQSPRKKSDAVSMLSECSTID

SEQ ID NO: 68 - protein, 9 aa, synthetic construct
VPIAVGESD

SEQ ID NO: 69 - protein, 5 aa, synthetic construct
PGESD

SEQ ID NO: 70 - protein, 25 aa, synthetic construct
PCAKVRPYIAEGESDTDSDLCTPCG

SEQ ID NO: 71 - protein, 25 aa, synthetic construct
SWMREKRYLAEGETDTDTDPFTPSG

SEQ ID NO: 72 - protein, 35 aa, synthetic construct
PGESDLEIMNTEELSSDSDSDYSKEKRNRSSSSEC

SEQ ID NO: 73 - protein, 19 aa, synthetic construct
PGESDLEIMNTEELSSDSD

SEQ ID NO: 74 - protein, 20 aa, synthetic construct
SDYSKEKRNRSSSSECSTVD

SEQ ID NO: 75 - protein, 30 aa, synthetic construct
LEIMNTEELSSDSDSDYSKEKRNRSSSSEC